(19) United States
(12) Patent Application Publication
(10) Pub. No.: US 2021/0155895 A1
(43) Pub. Date: May 27, 2021

(54) Pluripotent Stem Cell-Derived 3D Retinal Tissue and Uses Thereof

(71) Applicant: LINEAGE CELL THERAPEUTICS, INC., CARLSBAD, CA (US)

(72) Inventors: Igor Olegovich Nasonkin, Alameda, CA (US); Ratnesh Singh, Dublin, CA (US); Michael D. West, Mill Valley, CA (US); Hal Sterberg, Berkeley, CA (US); David LAROCCA, Alameda, CA (US)

(73) Assignee: LINEAGE CELL THERAPEUTICS, INC., CARLSBAD, CA (US)

(21) Appl. No.: 16/090,871

(22) PCT Filed: Apr. 4, 2017
(86) PCT No.: PCT/US2017/026016

§ 371 Date: Oct. 3, 2018

Related U.S. Application Data
(60) Provisional application No. 62/318,210, filed on Apr. 4, 2016.
(60) Provisional application No. 62/354,806, filed on Jun. 26, 2016.
(60) Provisional application No. 62/465,759, filed on Mar. 1, 2017.

(51) Int. Cl. C12N 5/00; C12N 5/079

(52) U.S. Cl. CPC C12N 5/0062; C12N 2513/00; C12N 5/0621

(57) ABSTRACT

Pluripotent stem cell-derived 3D retinal organoid compositions and methods of making using the same are disclosed.

### CROSS-REFERENCE TO RELATED APPLICATIONS

This application claims priority to, and the benefit of, U.S. provisional patent application Ser. No. 62/318,210 filed on Apr. 4, 2016, incorporated herein by reference in its entirety, U.S. provisional patent application Ser. No. 62/354,806 filed on Jun. 26, 2016, incorporated herein by reference in its entirety, and U.S. provisional patent application Ser. No. 62/465,759 filed on Mar. 1, 2017, also incorporated herein by reference in its entirety.

### STATEMENT OF GOVERNMENT SUPPORT

This invention was made with Government support under P30 EY008098 awarded by the National Institutes of Health. The Government has certain rights in the invention.

### FIELD

The present disclosure relates to the field of stem cell biology. More specifically, the present disclosure relates to pluripotent stem cell-derived 3D retinal tissue (organoid) compositions and methods of making and using the same.

### BACKGROUND

Partial or complete vision loss is a costly burden on our society. An estimated annual total financial cost of major adult visual disorders is $35.4 billion ($16.2 billion in direct medical costs, $11.1 billion in other direct costs, and $8 billion in productivity losses) and the annual governmental budgetary impact is $13.7 billion (Rein, D. B., et al., The economic burden of major adult visual disorders in the United States. Arch Ophthalmol, 2006. 124(12): p. 1754-60). There are several major causes of blindness in people, which result from photoreceptor (PR) cell death. Retinal degenerative (RD) diseases, which ultimately lead to the degeneration of PRs, are the third leading cause of worldwide blindness (Pascolini, D., et al., 2002 global update of available data on visual impairment: a compilation of population-based prevalence studies. Ophthalmic Epidemiol, 2004. 11(2): p. 67-115). Age-Related Macular Degeneration (AMD) is a leading cause of RD in people over 55 years old in developed countries. The “baby boom” generation of Americans is aging, and many of them will develop AMD, with the number of new AMD cases projected to nearly double by 2030. About 15 million people in the US are currently affected by AMD (Friedman, D. S., et al., Prevalence of age-related macular degeneration in the United States. Arch Ophthalmol, 2004. 122(4): p. 564-72; Jager, R. D., et al., Age-related macular degeneration. N Engl J Med, 2008. 358(24): p. 2606-17). AMD accounts for about 50% of all vision loss in the US and Canada (Access Economics, prepared for AMD Alliance International: The Global Economic Cost of Visual Impairment. 2010; Brandt, N., R. Vierk, and G. M. Rune, Sexual dimorphism in estrogen-induced synaptogenesis in the adult hippocampus. Int J Dev Biol, 2013. 57(5): p. 351-6). Therefore, AMD represents a major health issue facing the world and finding a treatment for it is of great significance. Retinitis pigmentosa (RP) is the most frequent cause of inherited visual impairment, with a prevalence of 1:4000, and is estimated to affect 50,000 to 100,000 people in the United States and approximately 1.5 million people worldwide (Christensen, R., Z. Shao, and D. A. Colon-Ramos, The cell biology of synaptic specificity during development. Curr Opin Neurobiol, 2013. 23(6): p. 1018-26; Hartong, D. T., E. L. Berson, and T. P. Dryja, Retinitis pigmentosa. Lancet, 2006. 368(9549): p. 1795-809).

There are currently two main strategies for restoration of vision loss resulting from retinal degeneration: (1) stem cell grafts, and (2) regeneration of cells in the human retina. The success of both approaches vitally depends on reestablishing the specific synaptic connectivity between the newly introduced (via regeneration or transplantation) retinal neurons and the remaining retinal neurons in the degenerating retina. Our lack of understanding of the mechanisms driving regeneration and reconnection of human retinal neurons hampers the development of therapies alleviating blindness. Furthermore, addressing such questions one mechanism or pathway at a time using animal, e.g. mouse, models is time consuming, costly and problematic in that the animal models do not always correctly recapitulate the pathways regulating development and synaptogenesis in the human retina (e.g. RB or retinoblastoma pathway).

While cell replacement is the ultimate goal of retinal cell therapies, many challenges to PR replacement, and neuronal replacement in general, remain (Nasonkin, I., et al., Long-term, stable differentiation of human embryonic stem cell-derived neural precursors grafted into the adult mammalian neostriatum. Stem Cells, 2009. 27(10): p. 2414-26; Hambright, D., et al., Long-term survival and differentiation of retinal neurons derived from human embryonic stem cell lines in un-immunosuppressed mouse retina. Mol Vis, 2012. 18: p. 920-36; Yao, J., et al., XIAP therapy increases survival of transplanted rod precursors in a degenerating host retina. Invest Ophthalmol Vis Sci, 2011. 52(3): p. 1567-72; Lamba, D., M. Karl, and T. Reh, Neural regeneration and cell replacement: a view from the eye. Cell Stem Cell, 2008. 2(6): p. 538-49; Lamba, D. A., M. O. Karl, and T. A. Reh, Strategies for retinal repair: cell replacement and regeneration. Prog Brain Res, 2009. 175: p. 23-31; MacLaren, R. E., et al., Retinal repair by transplantation of photoreceptor precursors. Nature, 2006. 444(7116): p. 203-7; Homma, K., et al., Developing rods transplanted into the degenerating retina of Crx-knockout mice exhibit neural activity similar to native photoreceptors. Stem Cells, 2013. 31(6): p. 1149-59; Tabar, V., et al., Migration and differentiation of neural precursors derived from human embryonic stem cells in the rat brain. Nat Biotechnol, 2005. 23(5): p. 601-6; Freed, C. R., et al., Do patients with Parkinson's disease benefit from embryonic dopamine cell transplantation? J Neurol, 2003. 250 Suppl 3: p. 11144-6; Bjorklund, A., et al., Neural transplantation for the treatment of Parkinson's disease. Lancet Neurol, 2003. 2(7): p. 437-45).

Ophthalmology research has recently uncovered significant problems originating from using oversimplified retinal tissue culture models without rechecking the result in more complex tissue (Krishnamoorthy, R. R., et al., A forensic path to RGC-5 cell line identification: lessons learned. Invest Ophthalmol Vis Sci, 2013. 54(8): p. 5712-9). Mouse models frequently cannot recapitulate the pathway driving disease progression in human retina (Macpherson, D., Insights from mouse models into human retinoblastoma. Cell Div, 2008. 3: p. 9.; Donovan, S. L., et al., Compensation by tumor suppressor genes during retinal development in mice and humans. BMC Biol, 2006. 4: p. 14.238).

Repairing the retina by functional cell replacement via cell transplantation or by inducing regeneration (which will work in cases of slowly progressing RD) is a complex task. In the case of neural retina, the task is especially challenging, because the new cells need to migrate to specific neuroanatomical locations in the retinal layer and re-establish specific synaptic connectivity in the synaptic architecture of the host retina. Synaptic remodeling of neural circuits during advancing retinal degeneration further complicates this task. With the exception of anti-VEGF antibody (Ab) injection therapy, there are no drugs yet that can substantially postpone, let alone repair, retinal damage in all major medical conditions leading to blindness. Preserving the original neural architecture of the retina, preserving the retinal pigmented epithelium (RPE)-photoreceptor (PR) niche, preserving the PR-2nd order retinal neuron niche and enhancing synaptic connectivity are major therapeutic goals in alleviating RP and AMD-related blindness. Until it is possible to regenerate human retina or to reconnect grafted PRs/retinal tissue, the strategy of slowing down PR cell death and deterioration of RPE-PR and PR-2nd order retinal neuron niches will remain the most viable alternative for reversing blindness. Moreover, for a number of RD diseases with rapid loss of PRs the strategy of retinal regeneration and likely PR grafting is unsuccessful, due to rapid deterioration of RPE-PR and PR-2nd order neuron niches. Thus, there is a need to develop new neuroprotective molecular treatments (e.g., small molecules, genes) and their combinations to efficiently protect photoreceptors from rapid deterioration and cell death.

There is a need for new therapeutics for the treatment of retinal degeneration (RD) in humans. Further, to improve our understanding of retinal degeneration in humans and to speed up discovery of novel drugs, factors, signaling molecules and pathways that provide PR neuroprotection and stimulation of synaptogenesis, there is a need for high-throughput, rapid screening methods and systems for evaluating a large number of candidate molecules that play a role in RD, and that correctly recapitulate processes of development and synaptogenesis in human retina. The present disclosure provides methods and compositions that address these needs.

### SUMMARY

Disclosed herein are methods for making in vitro retinal tissue from pluripotent cells; compositions comprising in vitro retinal tissue made from pluripotent cells; and methods of using in vitro retinal tissue for therapy and screening. The pluripotent cell-derived, three-dimensional in vitro retinal tissue disclosed herein is suitable for transplantation in cell-based therapies for retinal degeneration, and is an ideal tissue model to use in a discovery-based screening approach because it preserves the complexity of the RPE-PR-2nd order neuron niche while allowing for exceptional flexibility in experimental setup (e.g., genetic modification, rapid screening).

Accordingly, disclosed herein is a pluripotent cell-derived in vitro three-dimensional retinal tissue (i.e., a retinal organoid). Due to its growth and differentiation in adherent culture, the in vitro retinal tissue has a three-dimensional disc-like shape (i.e., similar to a flattened right cylinder) and has a laminar structure containing concentric layers of tissue extending out radially from a core of retinal pigmented epithelial (RPE) cells, as follows: a layer of retinal ganglion cells (RGCs), a layer of second-order retinal neurons (i.e., inner nuclear layer, INL), a layer of photoreceptor (PR) cells, and an exterior layer of retinal pigmented epithelial cells.

In certain embodiments, any one or more of the aforementioned layers has a thickness of one cell. In additional embodiments, any one or more of the layers has a thickness greater than a single cell. Any one of the layers can contain progenitor cells, in addition to the differentiated retinal cells present in the layer. Thus, for example, the RGC layer can also contain RGC progenitor cells; the inner nuclear layer can also contain progenitors of second-order retinal neurons; the photoreceptor (PR) cell layer can also contain PR progenitor cells, and the exterior RPE layer, and/or the RPE cell core, can also contain RPE progenitors. Any of the layers can also contain less differentiated progenitor cells (e.g., neuroectoderm progenitors, eye field progenitors, etc.).

In vitro retinal tissue, as disclosed herein, contains cells that express the adult stem cell marker LGR5 and/or TERT.

In certain embodiments, in vitro retinal tissue as disclosed herein contains cells that express one or more genes selected from the group consisting of RAX, OTX2, LHX2, CHX10, MITF, PAX6, CRX, Recoverin (RCVRN) and BRN3A.

In certain embodiments, in vitro retinal tissue as disclosed herein contains cells that express one or more of the SOX1, SOX2, OTX2 and FOXG1 genes.

In certain embodiments, in vitro retinal tissue as disclosed herein contains cells that express one or more of the RAX, LHX2, SIX3, SIX6 and PAX6 genes.

In certain embodiments, in vitro retinal tissue as disclosed herein contains cells that express one or more of the NEURO-D1, ASCL1 (MASH1), CHX10 and IKZF1 genes.

In certain embodiments, in vitro retinal tissue as disclosed herein contains cells that express one or more genes selected from the group consisting of CRX, RCVRN, NRL, NR2E3, RHO, PDE6B, PDE6C, OPN1MW, THRB(Thr2), CAR and OPN1SW.

In certain embodiments, in vitro retinal tissue as disclosed herein contains cells that express one or more genes selected from the group consisting of MAP2, DCX, ASCL1 and NEUROD1.

In certain embodiments, in vitro retinal tissue as disclosed herein contains cells that express one or more genes selected from the group consisting of MATH5, ISL1, BRN3A, BRN3B, BRN3C and DLX2.

In certain embodiments, in vitro retinal tissue as disclosed herein contains cells that expresses one or more genes selected from the group consisting of PROX1, PRKCA, CALB1 and CALB2.

In certain embodiments, in vitro retinal tissue as disclosed herein contains cells that express one or more genes selected from the group consisting of MITF, BEST1 (VMD2), TYR, TYRP, RPE65, DCT, PMEL, EZRIN and NHERF1.

In certain embodiments, in vitro retinal tissue as disclosed herein contains cells that express one or more genes selected from the group consisting of BDNF, GDNF, NGF, CNTF, PEDF (SERPIN-F1), VEGFA and FGF2.

In certain embodiments, in vitro retinal tissue as disclosed herein contains cells that express one or more genes selected from the group consisting of DICER, DROSHA, LIN28, DGCR8 (PASHA), AGO2 and TERT.

In certain embodiments, in vitro retinal tissue as disclosed herein contains cells that express one or more genes selected from the group consisting of Synaptophysin (SYP) and NF200.

In certain embodiments, in vitro retinal tissue as disclosed herein contains cells that do not express the NANOG and OCT3/4 genes.

In certain embodiments, in vitro retinal tissue as disclosed herein contains cells that do not express markers of endoderm, mesoderm, neural crest, astrocytes or oligodendrocytes.

Also provided are compositions comprising the in vitro retinal tissue as disclosed herein. Such compositions can comprise cell cultures and therapeutic compositions. Cell cultures comprising in vitro retinal tissue can also contain culture medium, mitogens, antibiotics, amino acids, hydrogels, etc. An exemplary hydrogel is HyStem® (BioTime, Alameda, Calif.). Cell cultures can also contain biological substrates deposited on the culture vessel (e.g., to promote adhesion of cells to the culture vessel), such that culture is conducted under adherent conditions. Exemplary substrates promoting adherence include, but are not limited to, Matrigel®, Matrigel®-GFR, vitronectin, laminin, fibronectin, collagen, gelatin, polyornithine and polylysine.

Therapeutic compositions can comprise in vitro retinal tissue and a delivery vehicle such as a pharmaceutically acceptable carrier or excipient.

Also provided are methods for making in vitro retinal tissue, wherein the methods comprise (a) culturing pluripotent cells, under adherent conditions, in the presence of noggin for a first period of time; then (b) culturing the adherent cells of (a) in the presence of noggin and basic fibroblast growth factor (bFGF) for a second period of time; then (c) culturing the adherent cells of (b) in the presence of Noggin, bFGF, Dickkopf-related protein 1 (Dkk-1) and insulin-like growth factor-1 (IGF-1) for a third period of time; and then (d) culturing the adherent cells of (c) in the presence of Noggin, bFGF, and fibroblast growth factor-9 (FGF-9) for a fourth period of time.

In some embodiments, the concentration of noggin is between 50 and 500 ng/ml; the concentration of bFGF is between 5 and 50 ng/ml; the concentration of Dkk-1 is between 5 and 50 ng/ml; the concentration of IGF-1 is between 5 and 50 ng/ml and the concentration of FGF-9 is between 5 and 50 ng/ml. In certain embodiments, the concentration of noggin is 100 ng/ml; the concentration of bFGF is 10 ng/ml; the concentration of Dkk-1 is 10 ng/ml; the concentration of IGF-1 is 10 ng/ml and the concentration of FGF-9 is 10 ng/ml.

In some embodiments, the first period of time is between 3 and 30 days; the second period of time is between 12 hours and 15 days; the third period of time is between 1 and 30 days; and the fourth period of time is 7 days to one year. In certain embodiments, the first period of time is 14 days; the second period of time is 14 days; the third period of time is 7 days; and the fourth period of time is 7 days to 12 weeks. In certain embodiments, the fourth period of time can last up to one year.

In certain embodiments for making in vitro retinal tissue, pluripotent cells are initially cultured in a first medium that supports stem cell growth and, beginning at two to sixty days after initiation of culture, a second medium that supports growth of differentiated neural cells is substituted for the first medium at gradually increasing concentrations until the culture medium contains 60% of the second medium and 40% of the first medium.

In some embodiments, the first medium is Neurobasal® medium and the second medium is Neurobasal®-A medium. In certain embodiments, the second medium is substituted for the first medium beginning seven days after initiation of culture. In certain embodiments, the culture medium contains 60% of the second medium and 40% of the first medium at 6 weeks after initiation of culture.

Conditions for adherent culture, used in the methods for making in vitro retinal tissue, comprise deposition of a substrate on a culture vessel prior to culture of the cells. Optionally, additional substrate is added during the first, second, third and/or fourth periods of time. Exemplary substrates include, but are not limited to, Matrigel®, Matrigel®-GFR, vitronectin, laminin, fibronectin, collagen, gelatin, polyornithine and polylysine.

In some embodiments, the fourth period of time is between 3 months and one year. In these embodiments, the method can further comprise addition of a biological substrate to the culture, during the fourth period of time, to facilitate adherence. Exemplary substrates include, but are not limited to, Matrigel®, Matrigel®-GFR, vitronectin, laminin, fibronectin, collagen, gelatin, polyornithine and polylysine.

Pluripotent cells for use in the disclosed methods of making in vitro retinal tissue include any pluripotent cell that is known in the art including, but not limited to, embryonic stem (ES) cells (e.g., human ES cells, primate ES cells), primate pluripotent stem cells (pPS cells), and induced pluripotent stem cells (iPS cells).

Therapeutic compositions comprising in vitro retinal tissue as disclosed herein (optionally comprising a buffer, saline, a pharmaceutically acceptable carrier and/or an excipient) can be used in methods for treating retinal degeneration; e.g., as occurs in retinitis pigmentosa (RP) and/or age-related macular degeneration (AMD). Thus, therapeutic methods utilizing in vitro retinal tissue as disclosed herein are also provided. In said therapeutic methods, a retinal organoid, or a portion thereof, is administered to a subject suffering from retinal degeneration. In certain embodiments, in vitro retinal tissue (i.e., a retinal organoid or a portion thereof) is administered to the eye of the subject, either intravitreally or subretinally.

In certain embodiments, a slice of a retinal organoid, taken along a chord or a diameter of an approximately cylindrical organoid, is used for administration. Such a slice possesses a flat, ribbon-like shape containing layers of different retinal cells (i.e., RPE cells, PR cells, second-order INL cells, RGCs) in a form that engrafts easily without deteriorating.

In certain embodiments, in vitro retinal tissue, or a portion thereof, such as a slice of an organoid taken along a chord or a diameter, is administered together with a hydrogel such as, for example, HyStem®. In certain embodiments, the hydrogel may be modified, e.g. embedded with one or more trophic factors, mitogens, morphogens and/or small molecules.

Also provided are screening methods. Accordingly, in certain embodiments, in vitro retinal tissue (i.e., retinal organoids) whose cells contain a first exogenous nucleic acid are provided. The first exogenous nucleic acid comprises (a) a recoverin (RCVN) promoter; (b) sequences encoding a first fluorophore; (c) an internal ribosome entry site (IRES) or a self-cleaving 2A peptide from porcine teschovirus-1 (P2A) site (Kim et al., High Cleavage Efficiency of a 2A Peptide Derived from Porcine Teschovirus-1 in Human Cell Lines, Zebrafish and Mice. PLoS ONE, 2011, Vol. 6 (4): e18556) for bicistronic exression; and (d) sequences encoding a fusion polypeptide comprising an anterograde marker and a second fluorophore. In certain embodiments, the first fluorophore is mCherry. In certain embodiments, the anterograde marker is wheat germ agglutinin (WGA). In certain embodiments, the second fluorophore is enhanced green fluorescent protein (EGFP). In retinal organoids containing the first exogenous nucleic acid, the second fluorophore (e.g., EGFP) is expressed in a PR cell (by virtue of the PR cell-specific RCVRN promoter), and is transported along the PR cell axon and into the cell with which the PR cell synapses (by virtue of the anterograde marker). Thus, retinal organoids containing the first exogenous nucleic acid can be used to measure synaptic activity of PR cells, as well as to measure the effects of substances that modulate synaptic activity of PR cells, by measuring transport of the second fluorophore into non-PR cells.

In certain embodiments, in vitro retinal tissue (i.e., retinal organoids) whose cells contain a second exogenous nucleic acid are provided. The second exogenous nucleic acid comprises (a) a tetracycline-inducible recoverin (RCVN) promoter (tet-on pRCVRN); (b) sequences encoding a test gene or a portion thereof; (c) an internal ribosome entry site (IRES); and (d) sequences encoding a marker gene. In certain embodiments, the marker gene is enhanced cyan fluorescent protein (ECFP). In certain embodiments, the test gene or portion thereof is inserted into the second exogenous nucleic acid using flippase recognition target (Frt) sequences present in the second exogenous nucleic acid.

Either of the first or second, or both, exogenous sequences can be chromosomally integrated. Alternatively, either of the first or second, or both, exogenous sequences can be extrachromosomal. In certain embodiments, one of the exogenous sequences is chromosomally integrated, and the other is extrachromosomal.

In certain embodiments, a method is provided for screening for a test substance that enhances synaptic connectivity between retinal cells, the method comprising (a) incubating in vitro retinal tissue whose cells comprise the first exogenous nucleic acid in the presence of the test substance; and (b) testing for synaptic activity; wherein an increase in synaptic activity in cultures in which the test substance is present, compared to cultures in which the test substance is not present, indicates that the test substance enhances synaptic connectivity. In certain embodiments, the method is used to screen for synaptic connections between PR cells and second-order retinal neurons.

Any substance can be used as a test substance. Exemplary test substances include, but are not limited to, exosome preparations, conditioned media, proteins, polypeptides, peptides, low molecular weight organic molecules, and inorganic molecules. Exosomes can be obtained from pluripotent cells or from various types of progenitor cells, such as those described in West et al. (2008) Regen Med 3:287 and US Patent Application Publication Nos. 20080070303 20100184033, all of which are incorporated herein by reference. Methods of obtaining exosome preparations from human embryonic progenitor cells are described, e.g. in US Patent Application Publication No. 20160108368, incorporated herein by reference.

Photoreceptor (PR) cells comprising the first exogenous nucleic acid express both the first and second fluorophores by virtue of the RCVRN promoter. Cells onto which PR cells form synapses express the second fluorophore by virtue of its anterograde transport to the post-synaptic cell. Thus, in certain embodiments, synaptic activity is determined by measuring the number of cells which express the second fluorophore, but do not express the first fluorophore.

In certain embodiments, synaptic activity is determined by electrical activity (e.g., as measured by patch-clamp methods), spectral changes in a calcium (Ca²⁺)-sensitive dye, spectral changes in a potassium (K⁺)-sensitive dye and/or by spectral changes in a voltage-sensitive dye.

Also provided are methods for assaying a test gene, or portion thereof, for its effect on synaptic activity utilizing cells comprising the second exogenous nucleic acid. Accordingly, in certain embodiments, a method for screening for a gene (or portion thereof) whose product enhances synaptic connectivity between retinal cells comprises (a) incubating in vitro retinal tissue whose cells comprise the second exogenous nucleic acid under conditions such that the test gene (or portion thereof) is expressed; and (b) testing for synaptic activity; wherein an increase in synaptic activity in cultures in which the test gene is expressed, compared to cultures in which the test gene is not expressed, indicates that the test gene encodes a product that enhances synaptic connectivity.

In certain embodiments, the conditions such that the test gene is expressed constitute culture in the presence of doxycycline or tetracycline.

In certain embodiments, the method is used to screen for the effect of a gene product (or portion thereof) on synaptic connections between PR cells and second-order retinal neurons.

In certain embodiments, synaptic activity is determined by electrical activity (e.g., as measured by patch-clamp methods), spectral changes in a calcium (Ca²⁺)-sensitive dye, spectral changes in a potassium (K⁺)-sensitive dye and/or by spectral changes in a voltage-sensitive dye.

If the cells comprising the second exogenous nucleic acid also comprise the first exogenous nucleic acid, synaptic activity can be determined by measuring the number of cells which express the second fluorophore (encoded by the first exogenous nucleic acid), but do not express the first fluorophore (encoded by the first exogenous nucleic acid).

Methods for screening for test substances (or test genes or portions thereof) that modulate PR cell survival are also provided. Accordingly, in certain embodiments, in vitro retinal tissue (i.e., retinal organoids) whose cells contain a mutation in the PDE6B or RHO gene are provided. Mutations in either gene lead to PR cell degeneration and death. Cells containing a mutation in the PDE6B or RHO gene can also comprise one or both of the first and second exogenous nucleic acids described above.

Thus, in certain embodiments, methods for screening for a test substance that promotes survival of photoreceptor (PR) cells comprise (a) incubating in vitro retinal tissue whose cells contain a mutation in the PDE6B or RHO gene in the presence of the test substance; and (b) testing for PR cell survival; wherein an increase in PR cell survival in cultures in which the test substance is present compared to cultures in which the test substance is not present indicates that the test substance promotes survival of photoreceptor cells.

Any substance can be used as a test substance. Exemplary test substances include, but are not limited to, exosome preparations, conditioned media, proteins, polypeptides, peptides, low molecular weight organic molecules, and inorganic molecules. Exosomes can be obtained from pluripotent cells or from various types of progenitor cells, such as those described in West et al. (2008) Regen Med 3:287 and US Patent Application Publication Nos. 20080070303 and 20100184033, all of which are incorporated herein by reference. Methods of obtaining exosome preparations from human embryonic progenitor cells are described, e.g., in US Patent Application Publication No. 20160108368, incorporated herein by reference.

Additional substances that can be tested for their effect on PR cell survival include mitogens, trophic factors, epigenetic modulators (i.e., substances that modulate, for example, DNA methylation, DNA hydroxymethylation, histone methylation, histone acetylation, histone phosphorylation, histone ubiquitination and/or microRNA expression) and substances that induce hypoxia or otherwise modulate cellular metabolism.

If the organoids whose cells comprise the PDE6B or RHO mutation also comprise the first exogenous nucleic acid described above, tests for synaptic activity, based on expression of the first and second fluorophores encoded by the first exogenous nucleic acid, can also be conducted.

Also provided are methods for assaying a test gene, or portion thereof, for its effect on PR cell survival utilizing retinal organoids whose cells comprise a PDE6B or RHO mutation and the second exogenous nucleic acid. Accordingly, in certain embodiments, methods for screening for a gene (or portion thereof) whose product promotes survival of photoreceptor (PR) cells comprises (a) incubating in vitro retinal tissue whose cells comprise a mutation in the PDE6B or RHO gene and whose cells comprise the second exogenous nucleic acid under conditions such that the test gene is expressed and (b) testing for PR cell survival; wherein an increase in PR cell survival in cultures in which the test gene is expressed, compared to cultures in which the test gene is not expressed, indicates that the test gene encodes a product that promotes survival of photoreceptor cells.

In certain embodiments, the conditions in which the test gene is expressed constitute culture in the presence of doxycycline or tetracycline.

Genes that can be tested include those that encode mitogens, trophic factors, epigenetic modulators (i.e., substances that modulate, for example, DNA methylation, DNA hydroxymethylation, histone methylation, histone acetylation, histone phosphorylation, histone ubiquitination and/or microRNA expression) and genes that encode products that induce hypoxia or otherwise modulate cellular metabolism.

If the organoids whose cells comprise the PDE6B mutation and the second exogenous nucleic acid also comprise the first exogenous nucleic acid described above, tests for synaptic activity, based on expression of the first and second fluorophores encoded by the first exogenous nucleic acid, can also be conducted. Accordingly, in certain embodiments, PR cell survival is determined by the number of cells in the culture that express the second fluorophore and do not express the first fluorophore. In additional embodiments, PR cell survival is determined by spectral changes in a calcium (Ca²⁺)-sensitive dye, a potassium (K⁺)-sensitive dye, or a voltage-sensitive dye.

In various embodiments described herein, the present disclosure provides, inter alia, compositions and methods for screening novel drugs, factors, genes and signaling pathways involved in RD and/or maintenance of normal PR function. In certain embodiments, compositions and methods for screening novel drugs, factors, genes and signaling pathways for PR regeneration are provided. In certain embodiments, compositions and methods for screening novel drugs, factors, genes and signaling pathways for specific synaptic reconnection of PRs to non-PR second order retinal neurons are provided. In certain embodiments, the present disclosure provides compositions and methods for screening novel drugs, factors, genes and signaling pathways providing PR neuroprotection via trophic, epigenetic and/or metabolic changes induced in the PRs.

In certain embodiments, the present disclosure provides methods and compositions for identifying small molecule drug targets and/or large molecule biologics suitable for the treatment or amelioration of RD-related vision loss. In certain embodiments, the present disclosure provides methods and compositions for identifying epigenetic modulators of PR degeneration and/or regeneration. In certain embodiments, the present disclosure provides methods and compositions for identifying trophic factors modulating PR degeneration and/or regeneration. In certain embodiments, the present disclosure provides methods and compositions for identifying modulators of PR energy metabolism. In certain embodiments, the present disclosure provides methods and compositions for identifying signaling molecules modulating PR degeneration and/or regeneration.

In certain embodiments, the present disclosure provides a 3D human retinal model comprising pluripotent stem cell-derived 3D retinal organoids. In certain embodiments, the present disclosure provides a system for screening RD-related vision loss in humans, comprising pluripotent stem cell-derived 3D retinal organoids and various factors for screening. In certain embodiments, the pluripotent stem cell-derived 3D retinal organoids are engineered to stably or transiently express one or more transgenes of interest.

In certain embodiments, the present disclosure provides a method for obtaining stem cell-derived 3D retinal organoids, the method essentially comprising culturing hESC colonies according to the protocol outlined in FIG. 1 and described in Example 1.

In certain embodiments, the present disclosure provides a method of screening for novel drugs, factors, genes and signaling pathways involved in RD and/or maintenance of normal PR function, the method comprising: 1) obtaining pluripotent stem cell-derived 3D retinal organoids, and 2) combining the pluripotent stem cell-derived 3D retinal organoids with one or more factors of interest, wherein the pluripotent stem cell-derived 3D retinal organoids have all retinal layers (RPE, PRs, inner retinal neurons and retinal ganglion cells). In certain embodiments, the pluripotent stem cell-derived 3D retinal organoids are capable of synaptogenesis. In certain embodiments, the pluripotent stem cell-derived 3D retinal organoids are capable of axonogenesis.

In another embodiment, the present disclosure provides a method for treating a subject in need of therapy, comprising administering to the subject hESC-derived 3D retinal tissue. In some embodiments, the subject in need of therapy needs retinal repair. In some embodiments, the subject in need of therapy is human. In some embodiments, the hESC-derived 3D retinal tissue is administered in a biologically acceptable carrier or delivery system. In some embodiments, the delivery system comprises a hydrogel.

In another embodiment, the present disclosure provides a pharmaceutical composition comprising isolated hESC-derived 3D retinal tissue and a biologically acceptable carrier or delivery system. In some embodiments, the delivery system comprises a hydrogel.

Other embodiments and aspects are described infra.

### BRIEF DESCRIPTION OF THE DRAWINGS

FIG. 1 shows a schematic that outlines the procedure for obtaining 3D retinal tissue (retinal organoids) from hES cells. Also shown are photomicrographs of 3D retinal tissue cultures at 4, 5 and 6 weeks after initiation of culture

FIG. 2 shows expression patterns of genes in human fetal development.

FIG. 3 shows evaluation of the expression of retinal markers in hESC-3D retinal tissue.

FIG. 4 shows markers of retinal pigmented epithelium (RPE) in developing hESC-3D retinal tissue. qRT-PCR data is shown in the Table at the top. The panels below depict sections of 6-week-old hESC-3D retinal organoids immunostained for RPE markers, EZRIN and NHERF. The left panel is focused on one RPE cell within the organoid, which displays the presence of both EZRIN and NHERF markers, while the panel on the right shows the presence of pigmented cells (RPE) in such hESC-3D retinal tissue, mostly on the basal side, which also carries a layer of PRs.

FIG. 5 shows typical results of staining hESC-3D retinal tissue, between 6-8 weeks of development, for various photoreceptor (PR) cell markers. A large number of PRs are observed in the basal side adjacent to the RPE (the nuclear marker is CRX; the cytoplasmic marker is recoverin (RCVRN) and the outer/inner segment marker is the lectin Peanut Agglutinin (PNA). Second order retinal neurons (CALRETININ=CALB2) with developed axons on the apical side of hESC-3D retina are also present. Some CALB2⁺ neurons are still migrating from the basal side (purple arrow), the side of mitotic division and cell fate acquisition.

FIG. 6 shows developing retinal ganglion cells (green: BRN3B RGC nuclear marker, arrow; blue: DAPI, nuclear marker) in 6-8wk old hESC-3D retinal tissue.

FIG. 7 shows analysis of synaptogenesis and axonogenesis in developing hESC-3D retinal tissue. Synaptogenesis begins at about 6-8 weeks in some organoids; and continues to become more pronounced during the 3rd and 4th month of hESC-3D retinal tissue development.

FIG. 8 shows measurements of electrical activity in hESC-3D retinal tissue. Upper panel, top, left: infrared image of a retinal neuron in hESC-3D retinal tissue being recorded, the pipet is filled with Lucifer yellow (top, right) to prove that patch-clamp connection between the neuron and the pipet is created. Left panel, bottom: Voltage-step responses of a 12-week old inner retinal neuron (likely amacrine, based on the position in 3D tissue and the shape of cell body with multiple axons, shown with Lucifer yellow) in hESC-3D retinal tissue. The transient inward currents (arrows) induced shortly after the capacitive currents were voltage-gated Na⁺, where the slow decaying outward currents were voltage-gated K⁺ currents. Lower panel, qRT-PCR of hESC-3D retinal tissue at 6 weeks and 12 weeks, targets: voltage-gated channel genes SCNA1, SCN2A, KCNA1, KCNA6.

FIG. 9 shows images of hESC-3D retinal tissue developed from hESC line H1 (WA01) containing RPE cells around a mass of cells carrying retinal neurons.

FIG. 10 shows estimates of PR, second order neuron and RGC number in a 1 mm slice of hESC-derived retinal tissue.

FIG. 11 shows the karyotype of hESC line H1 (WA01) used for the derivation of 3D retinal tissue. A normal karyotype (46, X,Y) is observed.

FIG. 12 shows hESC colony H1 (WA01) transfected (Fugene 6) with plasmid EGFP-N1 (as a control to evaluate transfection efficiency). Between 2-4% of hESCs were positive for EGFP.

FIG. 13 shows results indicating successful generation of a 2 base-pair change in the Pde6a gene of mouse ES cells, by CRISPR-Cas9 engineering. The off-target mutation rate was reduced in this case by using a D10A (“single nickase”) mutant version of Cas9 (pSpCas9n(BB)-2A-Puro). Shen, B., et al., Efficient genome modification by CRISPR-Cas9 nickase with minimal off-target effects. Nat Methods, 2014. 11(4): p. 399-402.

FIG. 14 shows expression of WGA-cre in HEK293 cells. The mCherry-IRES-WGA-Cre plasmid was tested for ability to express WGA-Cre in HEK293 cells by (i) transfecting it into HEK293, mCherry and Cre co-localization (upper three panels) and (ii) checking Cre activity by co-transfecting it with plasmid, expressing a conditional reporter CMV-loxp-STOP-loxP-YFP (lower three panels). Cre activates YFP.

FIG. 15 shows a comparison between transplantation of tubular, suspension culture-derived retinal tissue (panels A-C) and linear pieces of retinal tissue (panels D-G).

FIG. 16 shows a micrograph of a retinal organoid (upper left) showing how a linear slice of tissue can be cut from the organoid and transplanted (lower left). A schematic diagram of the shape and cellular composition of the slice is presented on the right. RGCs: retinal ganglion cells; RPE: retinal pigmented epithelium.

FIG. 17 shows expression of Lgr5 and TERT in a retinal organoid. Panels A and B show expression of TERT (green); panel C shows expression of Lgr5 (green). DAPI (blue) is a nuclear marker.

FIG. 18A and FIG. 18B show schematic diagrams of an exemplary in vitro retinal organoid, in which the three-dimensional shape of the organoid is approximated as a right cylinder. FIG. 18A shows a side view (also including a culture vessel); FIG. 18B shows a top view. Ovals represent retinal cells, with each color representing a different cell type. The large brown central oval represents a core of retinal pigmented epithelial (RPE) cells. Also shown is an exemplary method of obtaining a tissue slice from the organoid by cutting along a chord of the cylinder (red line).

FIG. 19 shows immunophenotyping results of 13-week old human fetal retina and 8-week old hESC-3D retinal tissue.

FIG. 20 shows a heat map illustrating the comparison of retinal progenitor cell expression profiles for hESC-3D retinal tissue (H1) and human fetal retina (F-Ret) at different time points.

FIG. 21 shows a heat map representing a comparison of RPE specific gene expression in hESC-3D retinal tissue versus human fetal retina at different time points.

FIG. 22 shows a heat map depicting the pattern of photoreceptor-specific gene expression, which is very similar in hESC-3D retinal tissue and human fetal retinal tissue.

FIG. 23 and FIG. 24 show heat maps that illustrate the similarities in gene expression profiles for amacrine cells and retinal ganglion cells (RGC) (respectively) among hESC-3D retinal tissue and human fetal retinal tissue at different time points.

FIG. 25 shows a heat map displaying similar cell surface marker gene expression profiles for hESC-3D retinal tissue and human fetal retinal tissue.

FIG. 26 shows images of the RPE and EZRIN cell markers which can be seen in the apical surface of both 10-week old human fetal retina and 8-week old hESC-3D retinal tissue.

FIG. 27 shows images of the distribution of OTX2 and MAP2 cell markers which are very similar in the 10-week old human fetal retina and 8-week old hESC-3D retinal tissue.

FIG. 28 show images of the pattern of cell marker distribution of the CRX (cone rod homeobox) marker, which is a major early photoreceptor marker, and the PAX6 marker for retinal progenitor cells and RGCs. The distribution patters in the 10-week old human fetal retina and 8-week old hESC-3D retinal tissue are comparable for these two markers.

FIG. 29 shows images of highly similar patterns of marker distribution for the Recoverin marker, which is present in young photoreceptors in the 13-week old human fetal retinal tissue and in 8-week old hESC-3D retinal tissue.

FIG. 30 shows images comparing the immunostaining of the BRN3B marker for RGCs in 10-week old human fetal retinal tissue and 8-week old hESC-3D retinal tissue.

FIG. 31 shows images of highly similar distribution patterns for cells labeled with CALB2 (calretinin) in 10-week old human fetal retinal tissue and 8-week old hESC-3D retinal tissue.

FIG. 32 shows the distribution of cells labeled with the LGR5 marker, which shows dividing stem cells (Wnt-signaling, postmitotic marker) for 10-week old human fetal retinal tissue and in 8-week old hESC-3D retinal tissue.

FIG. 33 provides a summary of the comparison of developmental dynamics in human fetal retina and human pluripotent stem cell derived retinal tissue.

FIG. 34a shows an Optical Coherence Tomography (OCT) image of the hESC-3D retinal tissue graft after 230 days.

FIG. 34b shows a graph of the results of visual acuity improvements testing using optokinetic (OKN) on rats at 2, 3, and 4 months after organoid transplantation surgery and control groups.

FIG. 34c shows a spike count heat map of visual responses in superior colliculus (electrophysiological recording) evaluated at 8.3 months post-surgery in one animal which demonstrated the animal's response to light. No responses to light were detected in RD age-matched control group and sham surgery RD group.

FIG. 34d shows a graph of examples of traces of visual responses in superior colliculus (electrophysiological recording).

FIG. 34e shows a table of visual responses in superior colliculus (electrophysiological recording) evaluated at 8.3 months post-surgery.

FIG. 34f through FIG. 34h show images demonstrating the presence of mature PRs and other retinal cell types in transplanted hESC-3D retinal tissue grafts.

### DETAILED DESCRIPTION

Before the present compositions and methods are described, it is to be understood that this invention is not limited to the particular processes, compositions, or methodologies described, as these may vary. It is also to be understood that the terminology used in the description is for the purpose of describing the particular versions or embodiments only, and is not intended to limit the scope of the present invention which will be limited only by the appended claims. Unless defined otherwise, all technical and scientific terms used herein have the same meanings as commonly understood by one of ordinary skill in the art. Any methods and materials similar or equivalent to those described herein can be used in the practice or testing of embodiments of the present disclosure.

### Definitions

The terms “hESC-derived 3D retinal tissue”, “hESC-derived 3D retinal organoids”, “hESC-3D retinal tissue,” “in vitro retinal tissue,” “retinal organoids,” “retinal spheroids” and “hESC-3D retinal organoids” are used interchangeably in the present disclosure and refer to pluripotent stem cell-derived three-dimensional aggregates comprising retinal tissue. The hESC-derived 3D retinal organoids develop all retinal layers (RPE, PRs, inner retinal neurons (i.e., inner nuclear layer) and retinal ganglion cells) and display synaptogenesis and axonogenesis commencing as early as around 6-8 weeks in certain organoids and becoming more pronounced at around 3^{rd} or 4^{th} month of hESC-3D retinal development. The 3D retinal organoids disclosed herein express the LGR5 gene, which is an adult stem cell marker. In addition, the hESC-derived 3D retinal organoids may be genetically engineered to transiently or stably express a transgene of interest.

Although the present disclosure refers to hESC-derived 3D retinal tissue, it will be appreciated by those skilled in the art that any pluripotent cell (ES cell, iPS cell, pPS cell, ES cell derived from parthenotes, and the like), may be used as a source of 3D retinal tissue according to methods of the present disclosure.

As used herein, “embryonic stem cell” (ES) refers to a pluripotent stem cell that is 1) derived from a blastocyst before substantial differentiation of the cells into the three germ layers; or 2) alternatively obtained from an established cell line. Except when explicitly required otherwise, the term includes primary tissue and established cell lines that bear phenotypic characteristics of ES cells, and progeny of such lines that have the pluripotent phenotype. The ES cell may be human ES cells (hES). Prototype hES cells are described by Thomson et al. (Science 282:1145 (1998); and U.S. Pat. No. 6,200,806), and may be obtained from any one of number of established stem cell banks such as UK Stem Cell Bank (Hertfordshire, England) and the National Stem Cell Bank (Madison, Wis., United States).

As used herein, “primate pluripotent stem cells” (pPS) refers to cells that may be derived from any source and that are capable, under appropriate conditions, of producing primate progeny of different cell types that are derivatives of all of the 3 germinal layers (endoderm, mesoderm, and ectoderm). pPS cells may have the ability to form a teratoma in 8-12 week old SCID mice and/or the ability to form identifiable cells of all three germ layers in tissue culture. Included in the definition of primate pluripotent stem cells are embryonic cells of various types including human embryonic stem (hES) cells, (see, e.g., Thomson et al. (1998) Science 282:1145) and human embryonic germ (hEG) cells (see, e.g., Shamblott et al. (1998) Proc. Natl. Acad. Sci. USA 95:13726); embryonic stem cells from other primates, such as Rhesus stem cells (see, e.g., Thomson et al., (1995) Proc. Natl. Acad. Sci. USA 92:7844), marmoset stem cells (see, e.g., (1996) Thomson et al., Biol. Reprod. 55:254), stem cells created by nuclear transfer technology (U.S. Patent Application Publication No. 2002/0046410), as well as induced pluripotent stem cells (see, e.g., Yu et al., (2007) Science 318:5858); Takahashi et al., (2007) Cell 131(5):861). The pPS cells may be established as cell lines, thus providing a continual source of pPS cells.

As used herein, “induced pluripotent stem cells” (iPS) refers to embryonic-like stem cells obtained by de-differentiation of adult somatic cells. iPS cells are pluripotent (i.e., capable of differentiating into at least one cell type found in each of the three embryonic germ layers). Such cells can be obtained from a differentiated tissue (e.g., a somatic tissue such as skin) and undergo de-differentiation by genetic manipulation which re-programs the cell to acquire embryonic stem cell characteristics. Induced pluripotent stem cells can be obtained by inducing the expression of Oct-4, Sox2, Kfl4 and c-Myc in a somatic stem cell. Thus, iPS cells can be generated by retroviral transduction of somatic cells such as fibroblasts, hepatocytes, gastric epithelial cells with transcription factors such as Oct-3/4, Sox2, c-Myc, and KLF4. Yamanaka S, Cell Stem Cell. 2007, 1(1):39-49; Aoi T, et al., Generation of Pluripotent Stem Cells from Adult Mouse Liver and Stomach Cells. Science. 2008 Feb. 14. (Epub ahead of print); 111 Park, Zhao R, West J A, et al. Reprogramming of human somatic cells to pluripotency with defined factors. Nature 2008; 451:141-146; K Takahashi, Tanabe K, Ohnuki M, et al. Induction of pluripotent stem cells from adult human fibroblasts by defined factors. Cell 2007; 131:861-872. Other embryonic-like stem cells can be generated by nuclear transfer to oocytes, fusion with embryonic stem cells or nuclear transfer into zygotes if the recipient cells are arrested in mitosis.

It will be appreciated that embryonic stem cells (such as hES cells), embryonic-like stem cells (such as iPS cells) and pPS cells as defined infra may all be used according to the methods of the present invention. Specifically, it will be appreciated that the hESC-derived 3D retinal organoids/retinal tissue may be derived from any type of pluripotent cells.

The term “subject,” as used herein includes, but is not limited to, humans, non-human primates and non-human vertebrates such as wild, domestic and farm animals including any mammal, such as cats, dogs, cows, sheep, pigs, horses, rabbits, rodents such as mice and rats. In some embodiments, the term “subject,” refers to a male. In some embodiments, the term “subject,” refers to a female.

The terms “treatment,” “treat” “treated,” or “treating,” as used herein, can refer to both therapeutic treatment or prophylactic or preventative measures, wherein the object is to prevent or slow down (lessen) an undesired physiological condition, symptom, disorder or disease, or to obtain beneficial or desired clinical results. In some embodiments, the term may refer to both treating and preventing. For the purposes of this disclosure, beneficial or desired clinical results may include, but are not limited to one or more of the following: alleviation of symptoms; diminishment of the extent of the condition, disorder or disease; stabilization (i.e., not worsening) of the state of the condition, disorder or disease; delay in onset or slowing of the progression of the condition, disorder or disease; amelioration of the condition, disorder or disease state; and remission (whether partial or total), whether detectable or undetectable, or enhancement or improvement of the condition, disorder or disease. Treatment includes eliciting a clinically significant response. Treatment also includes prolonging survival as compared to expected survival if not receiving treatment.

As used herein, the term “synaptic activity” refers to any activity or phenomenon that is characteristic of the formation of a synapse between two neurons. Synaptic activity can include electrical activity of a neuron, spectral changes in a voltage-sensitive or calcium-sensitive dye; and anterograde transport of a reporter such as, for example, wheat germ agglutinin (WGA).

### 3D Retinal Tissue (“Retinal Organoids”)

Using the methods and compositions disclosed herein, plupipotent cells (e.g., hESCs, iPS cells) can be converted to in vitro retinal tissue (“retinal organoids”). The derivation, growth and maturation of retinal organoids is conducted in adherent culture, rather than under embryoid body/retinosphere conditions. That is, in contrast to previous methods for deriving retinal tissue in suspension culture, resulting in the generation of ball-like optical cup structures, the methods disclosed in the present disclosure utilize adherent culture, which permits the generation of 3-dimensional flattened spheres, or “pancake-like” retinal tissue structures. Thus, this approach allows for derivation and growth of long, flat and rather flexible pieces of hESC-3D retinal tissue that are easily amenable to cutting for subretinal grafting. In contrast, optic cup-like spheres present a major problem for subretinal grafting. Such aggregates are rigid, cannot be cut as a long stretches of 3D retinal tissue (which is needed for retinal replacement therapies), and, as a consequence, can be delivered into subretinal space only when crumbled into small pieces, to fit into subretinal space niche. This leads to loss of 3D structure and tissue organization in grafted hESC-retina derived from optical cup-like structures.

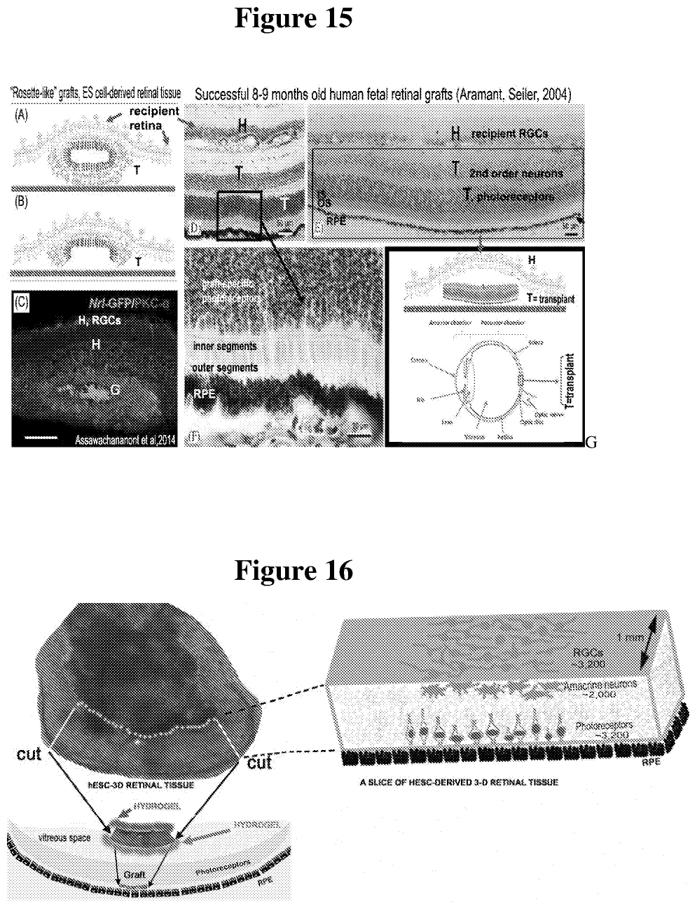

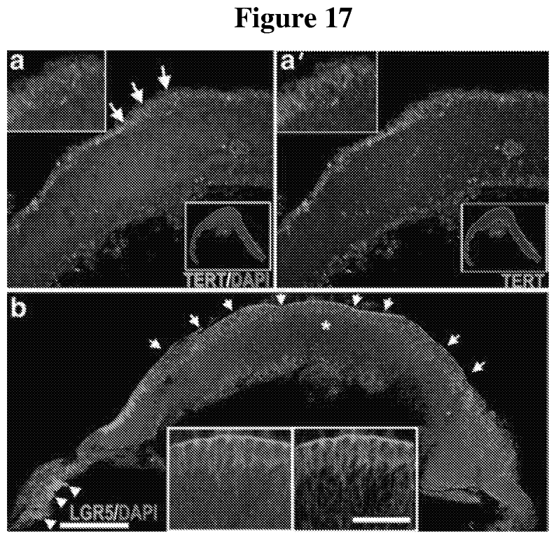

The therapeutic outcome (i.e., restoration of vision) of such therapy using retinal tissue from optical cup-like spheres is expected to be poor; due to poor structural integration of the crumbled optic cup-like tissue. This is illustrated in FIG. 15, which shows the poor result of grafting pieces of spherical hESC-retinal tissue (obtained from suspension culture) into the subretinal space of monkeys. Assawachananont et al. (2014) Stem Cell Reports 2: 662-674; see also Shirai et al. (2016) Proc. Natl. Acad. Sci. USA 113:E81-E90. Such grafts inevitably form tubular structures rather than a straight line of retinal tissue (as shown on the right side of FIG. 15, in which a long and flexible piece of human fetal retina was used for grafting into the subretinal space). Grafting as shown in the example on the right side of FIG. 15 resulted in improvements in vision in 7 out of 10 patients with subretinal grafts (Radtke et al., Vision improvement in retinal degeneration patients by implantation of retina together with retinal pigment epithelium. Am J Ophthalmol. 2008 146(2): 172-182).

Culture under adherent conditions, as disclosed herein, prevents the differentiating cells from forming spheres, as in previous methods of suspension culture, thereby allowing the in vitro retinal tissue (i.e., organoids) to attain a distinctive three-dimensional shape. Thus, in contrast to the tubular structures obtained using previous methods of deriving retinal tissue in suspension culture, the retinal organoids described herein, grown in adherent cultures, adopt a flattened cylindrical, disc-like, or “pancake-like” structure, allowing isolation of long and flexible pieces of hESC-derived 3D retinal tissue, resembling human fetal retina, for transplantation. Thus, the hESC-3D retinal tissue described herein is a good candidate to eventually replace human fetal tissue in all retinal replacement surgeries.

The in vitro retinal tissue of the present disclosure, in addition to possessing a disc-like or dome-like shape, is characterized by a laminar structure containing a plurality of layers of differentiated retinal cells and/or their progenitors. Each layer can be one cell thick or can contain multiple layers of cells.

In certain embodiments, three-dimensional in vitro retinal tissue, in the approximate shape of a flattened cylinder (or disc) contains a central core of retinal pigmented epithelial (RPE) cells, and, moving radially outward from the RPE cell core, a layer of retinal ganglion cells (RGCs), a layer of second-order retinal neurons (corresponding to the inner nuclear layer of the mature retina), a layer of photoreceptor (PR) cells, and an outer layer of RPE cells. Each of these layers can possess fully differentiated cells characteristic of the layer, and optionally can also contain progenitors of the differentiated cell characteristic of the layer. For example, the RPE cell layer (or core) can contain RPE cells and/or RPE progenitor cells; the PR cell layer can contain PR cells and/or PR progenitor cells; the inner nuclear layer can contain second-order retinal neurons and/or progenitors of second-order retinal neurons; and the RGC layer can contain RGCs and/or RGC progenitor cells.

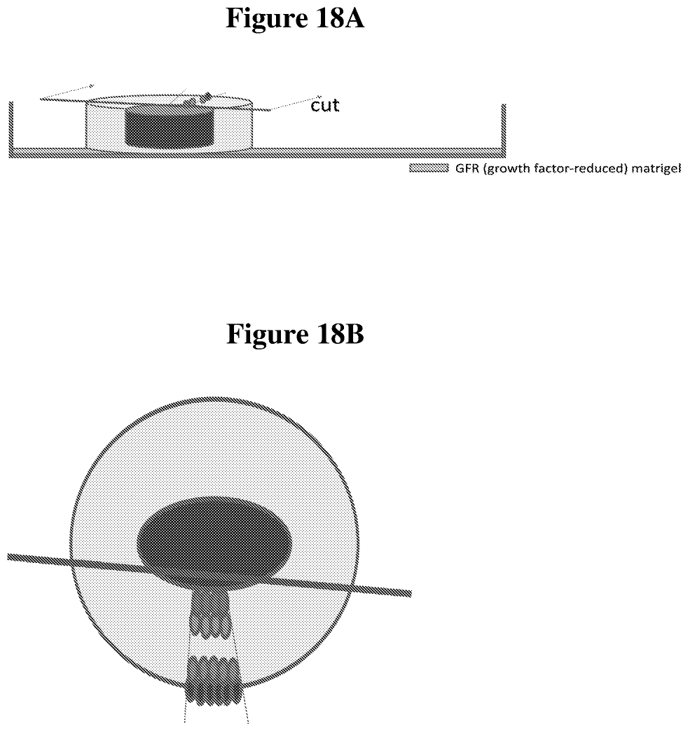

Due to the unique laminar structure of the in vitro retinal tissue disclosed herein (described above), it is possible to obtain slices from the three-dimensional organoid, (e.g., for transplantation) that contain layers of different retinal cells (e.g., RGCs, second order neurons, PR cells and RPE cells). Thus, if the shape of an in vitro retinal tissue disc as disclosed herein is approximated as a right cylinder, cutting along a diameter or along a chord of such a cylinder will yield a strip of tissue containing multiple cell layers. See FIGS. 18A and 18B. Not only will such a strip of tissue contain multiple cell layers (i.e., lamina); it will possess a flat, ribbon-like structure which facilitates transplantation and engraftment. Accordingly, in vitro retinal tissue as disclosed herein, or portions thereof, can be used for transplantation, for example in the treatment of retinal degeneration (see below).

In an exemplary method for deriving 3-D retinal organoids, pluripotent cells (e.g., hESCs, iPS cells) are cultured in the presence of the noggin protein (e.g., at a final concentration of between 50 and 500 ng/ml final concentration) for between 3 and 30 days. Basic fibroblast growth factor (bFGF) is then added to the culture (e.g., at a final concentration of 5-50 ng/ml) along with noggin, and culture is continued for an additional 0.5-15 days. At that time, the morphogens Dickkopf-related protein 1 (Dkk-1) and insulin-like growth factor-1 (IGF-1) (each at e.g., 5-50 ng/ml) are added to the culture, along with the noggin and bFGF already present, and culture is continued for an additional time period of between 1 and 30 days. At this point, Dkk-1 and IGF-1 are removed from the culture and fibroblast growth factor-9 (FGF-9) is added to the culture (e.g., at 5-10 ng/ml) along with noggin and bFGF. Culture is continued in the presence of noggin, bFGF and FGF-9 until retinal tissue is formed; e.g., from 1-52 weeks.

In certain embodiments for deriving 3-D retinal organoids, pluripotent cells (e.g., hESCs, iPS cells) are cultured in the presence of the noggin protein (at 100 ng/ml final concentration) for two weeks. Basic fibroblast growth factor (bFGF) is then added to the culture (to a final concentration of 10 ng/ml) along with noggin (at 100 ng/ml), and culture is continued for an additional two weeks. At that time, the morphogens Dickkopf-related protein 1 (Dkk-1) and insulin-like growth factor-1 (IGF-1) are added to the culture (each to a final concentration of 10 ng/ml), along with the noggin and bFGF already present, and culture is continued for an additional week. At this point, Dkk-1 and IGF-1 are removed from the culture and fibroblast growth factor-9 (FGF-9) is added to the culture (to a final concentration of 10 ng/ml) along with noggin and bFGF. Culture is continued in the presence of noggin, bFGF and FGF-9 until retinal tissue is formed. In certain embodiments, retinal tissue begins to appear within two weeks after addition of FGF-9 (i.e., 6 weeks after initiation of culture in noggin).

In addition to the polypeptide growth factors used in the manufacture of the in vitro retinal tissue as described above, modifications of said proteins and/or agonists or antagonists of the signaling pathways modulated by said proteins, can also be used.

Culture is conducted under adherent conditions to generate the three-dimensional in vitro retinal organoids disclosed herein. To achieve adherent culture conditions, in which the cells in culture adhere to the culture vessel, a biological substrate is applied to the culture vessel. For example, the surface of the culture vessel is coated with a biological substrate such as, for example, feeder cells, e.g. murine fibroblasts, Matrigel®, vitronectin, laminin, or fibronectin; and pluripotent cells (e.g., hESCs) are plated onto the substrate. In certain embodiments, culture is conducted in the presence of a hydrogel, e.g., HysStem®, or a modified hydrogel, e.g. a hydrogel embedded with one or more of trophic factors, morphogens and/or mitogens.

In certain embodiments, retinal tissue is detectable within six weeks after initiation of culture of pluripotent cells in the presence of noggin (or modified noggin or a noggin agonist). However, long-term culture can be continued from three months to up to one year, thereby providing a long-lasting source of in vitro retinal tissue. In certain embodiments, longer-term culture is facilitated by provision of additional substrate (e.g., MatriGel®) to the long-term culture, to maintain cell adherence to the culture vessel.

In the course of retinal organoid formation, hESCs differentiate into progenitor cells, which themselves undergo further differentiation into, e.g., phorotreceptor cells, second order neurons (e.g., amacrine cells), ganglion cells and retinal pigmented epithelium (RPE) cells. To support the growth and survival of these more differentiated cells, yet still preserve the stem cells and progenitor cells remaining in the cultures, the content of the culture medium is changed gradually over time, from a medium that supports survival of embryonic cells (e.g., Neurobasal®, also denoted Neurobasal®-E) to a medium that supports survival of more differentiated cells (e.g., Neurobasal®-A). Accordingly, in certain embodiments for the manufacture of in vitro retinal tissue, pluripotent cells are initially cultured in a first medium that supports stem cell growth and, beginning at two to sixty days after initiation of culture, a second medium that supports growth of differentiated neural cells is substituted for the first medium at gradually increasing concentrations. In certain embodiments, a second medium supporting differentiated cell growth is gradually substituted for a first medium that supports stem cell growth beginning seven days after initiation of culture, and continuing until the culture medium contains 60% of the second medium and 40% of the first medium.

In additional embodiments, for the first week of culture, the culture medium is 100% Neurobasal®; from 8-14 days after initiation of culture, the medium is changed to 97% Neurobasal®/3% Neurobasal®-A; from 15-21 days of culture, the medium is 93% Neurobasal®/7% Neurobasal®-A; from 21-28 days of culture, the medium is 85% Neurobasal®/15% Neurobasal®-A; from 29-35 days of culture, the medium is 70% Neurobasal®/30% Neurobasal®-A; and from day 36 onward, the medium is 40% Neurobasal®/60% Neurobasal®-A.

The retinal organoids disclosed herein express the adult stem cell marker LGR5. Barker et al. (2007) Nature 449:1003-1008. The Lgr5 protein is responsible for renewal and regeneration of cells in several tissue types, including retina. Chen et al. (2015) Aging Cell 14:635-643. In retinal organoids, it is generally co-expressed, with TERT, on the basal side of the organoids near the portion of the organoid occupied by RPE cells. See FIG. 17.

During the conversion of hESCs to retinal organoids, the hESCs differentiate into progenitor cells, which themselves differentiate further into mature retinal cells, such as photoreceptor (PR) cells, retinal ganglion cells (RGCs), cells of the inner nuclear layer (INL) and cells of the retinal pigmented epithelium (RPE). Thus, cells in organoid cultures express genes characteristic of these progenitor cells and mature retinal cells.

For example, in certain embodiments, cells in the retinal organoid express or more genes selected from the group consisting of RAX, OTX2, LHX2, CHX10, MITF, PAX6, CRX, Recoverin (RCVRN) and BRN3A.

In certain embodiments, cells in the organoid express a marker of neuroectoderm or anterior neuroectoderm selected from one or more of SOX1, SOX2, OTX2 and FOXG1.

In certain embodiments, cells in the organoid express a marker of the eye field selected from one or more of RAX, LHX2, SIX3, SIX6 and PAX6.

In certain embodiments, cells in the organoid express a marker of retinal progenitor cells selected from one or more of NEURO-D1, ASCL1 (MASH1), CHX10 and IKZF1.

In certain embodiments, cells in the organoid express a marker of photoreceptor cells selected from one or more of CRX, RCVRN, NRL, NR2E3, PDE6B, and OPN1SW.

In certain embodiments, cells in the organoid express a marker of ganglion cells selected from one or more of MATH5, ISL1, BRN3A, BRN3B, BRN3C and DLX2.

In certain embodiments, cells in the organoid express a marker of inner nuclear layer cells selected from one or more of PROX1, PRKCA, CALB1 and CALB2.

In certain embodiments, cells in the organoid express a marker of retinal pigmented epithelium selected from one or more of MITF, TYR TYRP, RPE65, DCT PMEL, EZRIN and NHERF1.

As cells differentiate in the retinal organoid cultures, they cease to express certain stem cell markers. Accordingly, in certain embodiments, cell in the retinal organoid do not express either or both of the NANOG and OCT3/4 genes.

The retinal organoid cells also do not express markers of endoderm, mesoderm, neural crest, astrocytes or oligodendrocytes.

Compositions comprising in vitro retinal tissue are also provided. For example, cell cultures comprising the in vitro retinal tissue disclosed herein are provided. Such cultures can contain culture medium (e.g., DMEM, NeuroBasal®, NeuroBasal-A® or any other medium known in the art). Cultures can also contain substrates, optionally applied to the culture vessel, that facilitate adherence of cells to the culture vessel. Exemplary substrates include, but are not limited to, fibroblasts, Matrigel®, vitronectin, laminin, and fibronectin. Cultures can also optionally contain a hydrogel such as, for example HyStem®.

Compositions comprising in vitro retinal tissue, or portions thereof, can also contain one or more pharmaceutically acceptable carriers or excipients, as are well-known in the art (see below).

### Therapeutic Uses of 3D Retinal Organoids

In certain embodiments, the 3D retinal organoids (i.e., in vitro retinal tissue) of the present disclosure can be used for maintenance, repair and regeneration of retinal tissue in any subject, including human or non-human subjects. To determine the suitability of compositions comprising 3D retinal organoids of the present disclosure for therapeutic administration, such compositions can first be tested in a suitable subject such as a rat, mouse, guinea pig, rabbit, cow, horse, sheep, pig, dog, primate or other mammal.

The 3D retinal organoids of the present disclosure may be used for repairing and/or regenerating retinal tissues in a human patient or other subject in need of cell therapy. In certain embodiments, one or more 3D retinal organoids, or portions thereof, are administered to a subject for the treatment of retinal degeneration in age-related macular degeneration (AMD) or retinitis pigmentosa (RP).

The 3D retinal organoids are administered in a manner that permits them to graft or migrate to the intended tissue site and reconstitute or regenerate the functionally deficient area. Therefore, in certain embodiments, one or more slices of 3D retinal organoid is transplanted to the eye of the subject; e.g., intravitreally or subretinally. As described supra, a slice cut from a retinal organoid along a diameter or a chord provides a flat, ribbon-like piece of tissue suitable for transplantation, and superior in its abilities to engraft and restore optical function. In certain embodiments, the 3D retinal organoid, or slice thereof, is administered together with a hydrogel. In these cases, the organoid can either be cultured in the presence of the hydrogel, or the hydrogel can be mixed with the organoid, or slice thereof, prior to administration. Exemplary hydrogels include, but are not limited to, HyStem®, and hydrogels described in U.S. Pat. Nos. 8,324,184, 8,859,523, 7,928,069, 7,981,871 and 8,691,793, incorporated herein by reference.

Administration of the 3D retinal organoids is achieved by any method known in the art. For example, the cells may be administered surgically directly to the eye, either intravitreally or subretinally. Alternatively, non-invasive procedures may be used to administer the 3D retinal organoids to the subject. Examples of non-invasive delivery methods include the use of syringes and/or catheters.

### Screening Using 3D Retinal Organoids

The 3D retinal organoids of the present disclosure can be used to screen for factors (such as gene products, small molecule drugs, peptides or other large molecule biologics, oligonucleotides, and/or epigenetic or metabolic modulators) or environmental conditions (such as culture conditions) that affect the characteristics of retinal cells, particularly PR cells. Characteristics may include phenotypic or functional traits of the cells. Other characteristics that may be observed include the differentiation status of the cells; the synaptic activity of the cells; the maturity of the cells and the survival and growth rate of the cells after exposure to the factor.

Thus the 3D retinal organoids may be contacted with one or more factors (i.e., test substances) and the effects of the factors may be compared to an aliquot of the same 3D retinal organoids that has not been contacted with the factors. Any factor or test substance can be screened according to the methods disclosed herein including, but not limited to, exosome preparations, conditioned media, proteins, polypeptides, peptides, low molecular weight organic molecules, and inorganic molecules. Exosomes can be obtained from pluripotent cells or from various types of progenitor cells, such as those described in West et al. (2008) Regen Med 3:287 and US Patent Application Publication Nos. 20080070303 20100184033, all of which are incorporated herein by reference. Methods of obtaining exosome preparations from human embryonic progenitor cells are described, e.g. in US Patent Application Publication No. 20160108368, incorporated herein by reference.

Other screening applications of this invention relate to the testing of pharmaceutical compounds for their effect on retinal cells, particularly PR cells. Screening may be done either because the compound is designed to have a pharmacological effect on the cells, or because a compound is designed to have effects elsewhere and may have unintended side effects on retinal cells. The screening can be conducted using any of the 3D retinal organoids of the present disclosure in order to determine if the target compound has a beneficial or harmful effect on retinal cells.

The reader is referred generally to the standard textbook In vitro Methods in Pharmaceutical Research, Academic Press, 1997. Assessment of the activity of candidate substances (e.g., pharmaceutical compounds) generally involves combining the 3D retinal organoids of the present disclosure with the candidate substance (e.g., gene product, chemical compound), either alone or in combination with other drugs. The investigator determines any change in the morphology, marker phenotype as described infra, or functional activity of the cells, that is attributable to the substance (compared with untreated cells or cells treated with an inert substance), and then correlates the effect of the substance with the observed change.

Where an effect is observed, the concentration of the substance can be titrated to determine the median effective dose (ED50).

Cytotoxicity can be determined in the first instance by the effect on cell viability, survival, morphology, and the expression of certain markers and receptors. Effects of a drug on chromosomal DNA can be determined by measuring DNA synthesis or repair. [³H]-thymidine or BrdU incorporation, especially at unscheduled times in the cell cycle, or above the level required for cell replication, is consistent with a drug effect. Expression of the Ki76 marker (e.g., increased Ki76 expression in the presence of a test substance) is an indicator of cell proliferation. Unwanted effects can also include unusual rates of sister chromatid exchange, determined by metaphase spread. The reader is referred to A. Vickers (pp. 375-410 in In vitro Methods in Pharmaceutical Research, Academic Press, 1997) for further elaboration.

Synaptic activity can be determined, for example, by observation of spectral changes in voltage-sensitive dyes introduced into cells, by electrical activity of cells (e.g., measured by patch-clamp techniques), by changes in spectral properties of Ca²⁺-sensitive and/or K⁺-sensitive dyes, and by observation of anterograde transport of a marker from one cell to another. In certain embodiments, wheat germ agglutinin (WGA) is used as an anterograde marker. In certain embodiments, WGA is fused to or labeled with a detectable molecule, so that transport can be observed via the detectable molecule. Detectable molecules include the various fluorescent proteins as known in the art (e.g., green fluorescent protein, red fluorescent protein, yellow fluorescent protein, cyan fluorescent protein, etc.), alkaline phosphatase, horseradish peroxidase, and radioactively labeled molecules.

In certain embodiments, photoreceptor (PR) cells in the retinal organoids disclosed herein express a transgene encoding a polypeptide comprising a fusion between WGA and a fluorescent polypeptide (e.g., EGFP), which serves as a marker for synaptic activity of PR cells. Expression of the fusion transgene is under the control of the PR-specific recoverin (RCVRN) promoter, so expression of the transgene is limited to PR cells. If a PR makes a synaptic connection with another cell (e.g., a second-order retinal neuron) the fusion protein travels down the PR cell axon and into the post-synaptic cell. Thus, fluorescence (e.g., green fluorescence in the case of a WGA/EGFP fusion protein) is observed in the post-synaptic partner of the PR cell. In certain embodiments, the cells comprising a, for example, WGA-EGFP transgene also express another fluorophore (e.g., mCherry) whose expression is limited to the PR cell. Sequences encoding the PR-specific fluorophore (e.g., mCherry) can be present in the same transgene construct that expresses the WGA-EGFP marker, or in a different transgene construct. Expression of the PR-specific fluorophore can also be placed under the control of the recoverin promoter, so that its expression is restricted to PR cells. In certain embodiments, both fluorophores are contained in the same transgene construct, which is introduced into pluripotent (e.g., hESC) cells prior to their conversion to retinal organoids. For example, a transgene construct containing, in operative linkage, a recoverin promoter (pRCVRN), sequences encoding the mCherry fluorophore, an internal ribosome entry site (IRES) and sequences encoding a wheat germ agglutinin (WGA)/enhanced green fluorescent protein (EGFP) fusion gene is introduced into hESCs prior to their conversion to retinal organoids. The transgene can be integrated or non-chromosomal.

For example, in organoids made from cells containing a pRCVRN-mCherry-IRES-WGA/EGFP transgene, synaptic activity of PR cells can be detected, since PR cells will exhibit both red fluorescence due to mCherry and green fluorescence due to EGFP; and their post-synaptic partners will exhibit only green (EGFP) fluorescence. Thus, in certain embodiments, formation of synapses, by PR cells, onto second-order retinal neurons, is detected.

It will be clear that the foregoing approach can be used to assess the synaptic activity of cells other that PR cells, simply be replacing, in the transgene construct, the PR cell-specific recoverin promoter with a promoter that is specific to the cell under study. That is, the mCherry-IRES-WGA/EGFP cassette can be placed under the transcriptional control of, for example, a RPE cell-specific promoter, an INL cell-specific promoter, a RG cell-specific promoter, etc. to assess the synaptic activity of RPE cells, INL cells and RG cells, respectively.

For applications in which it is desirable to test the effect of a predetermined gene product on survival and/or synaptic activity of PR cells, cells containing the first construct described above (i.e., the pRCVRN-mCherry-IRES-WGA/EGFP transgene) can also contain a second construct that allows conditional expression of a gene of interest. For example, in certain embodiments, hESCs used for generation of retinal organoids contain an exogenous nucleic acid comprising, in operative linkage, a tetracycline-inducible recoverin promoter (tet-on pRCVRN); sequences encoding a test gene; an internal ribosome entry site (IRES) or a self-cleaving 2A peptide from porcine teschovirus-1 (P2A) site (Kim et al., High Cleavage Efficiency of a 2A Peptide Derived from Porcine Teschovirus-1 in Human Cell Lines, Zebrafish and Mice. PLoS ONE, 2011, Vol. 6 (4): e18556) for bicistronic exression; and sequences encoding a marker gene, e.g., a fluorophore such as, e.g., enhanced cyan fluorescent protein (ECFP).

Accordingly, the present disclosure provides vectors (e.g., lentiviral) that contain a tetracycline-inducible recoverin promoter (tet-on pRCVRN); FLP recombinase target (Frt) sequences; an internal ribosome entry site (IRES); and sequences encoding a marker gene such as a fluorophore (e.g., ECFP). Such vectors are used for making constructs that conditionally express a test gene of interest in PR cells. For example, test sequences encoding a protein of interest or a portion thereof are introduced into the vector, at the Frt sites, using FLP-mediated recombination. Following insertion of the test sequences, this vector is introduced into pluripotent cells, which are then converted to in vitro retinal tissue using the methods disclosed herein. ECFP fluorescence can be assayed, if necessary, to confirm that tet- or dox-inducible gene expression is limited to PR cells.

Using the cells and constructs described above, the effect of a particular gene on synaptic activity is assessed, in retinal organoids made from cells containing both of the above-described constructs, by activating expression of the test gene using, e.g., doxycycline (DOX) and measuring, e.g., mCherry and EGFP fluorescence to determine synaptic connections between PR cells and their post-synaptic partners as described above. Alternatively, or in addition, electrical activity and/or spectral changes in voltage-sensitive and/or calcium-sensitive dyes can be used as indicators of synaptic activity. In certain embodiments, synaptic connections between PR cells and second-order retinal neurons are detected.

For determining the effect of a transgene on PR cell growth and/or proliferation, any of the methods described above and/or known in the art for measuring cell growth and proliferation can be used. In certain embodiments for measuring the effect of a transgene on PR cell growth and/or proliferation, the cells do not contain the pRCVRN-mCherry-IRES-WGA/EGFP transgene.

Introduction of transgenes such as those described above can be accomplished by any method for DNA integration known in the art, for example, lentiviral vectors or the CRISPR/Cas-9 system.

### Screening Using a PR Cell Degeneration Model in 3D Retinal Organoids

In certain embodiments, the retinal organoid system disclosed herein is used as a screening system to identify substances that prevent death and/or promote survival of PR cells. For this purpose, in certain embodiments, a mutation in the PDE6B gene is introduced into hES cells, which are then used for the derivation of in vitro retinal tissue as described herein. The hESCs can optionally contain the pRCVRN-mCherry-IRES-WGA/EGFP construct described above. Also, the hESCs can contain a tet-on pRCVRN-Frt-IRES-ECFP construct or a tet-on pRCVRN-(test gene)-IRES-ECFP construct as described above.

The PDE6B mutation is the human counterpart of the mouse rd10 mutation, which leads to PR cell degeneration and death. The RHO mutation is one of the most frequent mutations in patients with RD, causing blindness. Thus, in retinal tissue (i.e., organoids) made from hESCs containing a PDE6B or RHO mutation, PR cells are prone to degeneration and death. By incubating such organoids in the presence of one or more test substances, it is possible to determine whether the test substance reverses the death and degeneration of PR cells by assaying for viability, proliferation and synaptic activity of the PR cells.

Any method of mutagenesis known in the art can be used to introduce a PDE6B or RHO mutation into hESCs. For example, the CRISPR-Cas9 system, TALENS or zinc finger nucleases can be used. In one embodiment, the sequence ATCCAGTAG in exon 22 of the PDE6B gene is converted to ATCCTATAG.

In organoids containing the pRCVRN-mCherry-IRES-WGA/EGFP transgene, synaptic activity can be assessed by noting the presence and number of mCherry⁻/EGFP⁺ post-synaptic partners of PR cells. Thus, in certain embodiments, organoids whose cells contain a PDE6B or RHO mutation and a pRCVRN-mCherry-IRES-WGA/EGFP transgene are cultured in the presence of a test substance, and PR cell survival and synaptic activity are assessed.

If the organoids contain the tet-on pRCVRN-(test gene)-IRES-ECFP construct, the effect of the test gene on PR cell survival can be assayed by observing and/or assaying the organoids in the presence (e.g., + doxycycline) and absence (e.g., doxycycline) of the test gene product. Thus, in certain embodiments, organoids whose cells contain a tet-on pRCVRN-(test gene)-IRES-ECFP transgene are cultured in the presence and absence of doxycycline, and PR cell survival and synaptic activity are assessed. If the organoids additionally contain a pRCVRN-mCherry-IRES-WGA/EGFP, synaptic activity can be assessed by noting the presence and number of mCherry⁻/EGFP⁺ post-synaptic partners of PR cells. Alternatively, or in addition, synaptic activity can be assessed by electrical activity and/or spectral changes in voltage- and/or calcium-sensitive dyes. Thus, in certain embodiments, to identify gene products that promote PR cell survival, organoids whose cells contain both a pRCVRN-mCherry-IRES-WGA/EGFP construct and a tet-on pRCVRN-(test gene)-IRES-ECFP construct are cultured in the presence and absence of doxycycline, and PR cell survival and synaptic activity are assessed by noting, for example, the presence and number of mCherry⁻/EGFP⁺ post-synaptic partners of PR cells.

Methods for determining PR cell survival include, for example, evaluating PR cell number by immunohistochemistry, mCherry fluorescence, EGFP fluorescence spectral changes in voltage-sensitive and/or calcium-sensitive dyes and change in electric activity in organoids in response to light.

Candidate genes to be tested for the ability of their product to promote PR cell survival can be, for example, genes encoding mitogens (i.e., polypeptides that stimulate cell division) or trophic factors (e.g., polypeptides that stimulate cell growth and/or differentiation). Exemplary trophic factors and mitogens include brain-derived neurotrophic factor (BDNF), glial cell-derived neurotrophic factor (GDNF), nerve growth factor (NGF), neurotrophin 3 (NT3), basic fibroblast growth factor (bFGF), ciliary neurotrophic factor (CNTF), and pigment epithelium-derived factor (PEDF). In certain embodiments, a cDNA encoding one or more of the aforementioned factors is inserted into the pRCVRN-Flt-IRES-ECFP construct in the hESCs used for derivation of 3D retinal organoids.

Additional factors and/or test substances that can be assayed for their effect of PR cell survival include exosome preparations, conditioned media, proteins, polypeptides, peptides, low molecular weight organic molecules, and inorganic molecules. Exosomes can be obtained, for example, from pluripotent cells. Proteins and gene products that can be tested for their effect on PR cell survival include epigenetic modulators and molecules that induce hypoxia or that are associated with the hypoxic response, for example, HIF-1α. Epigenetic modulators include, for example, protein that modulate DNA methylation, DNA hydroxymethylation, histone methylation, histone acetylation, histone phosphorylation, histone ubiquitination and expression of chromatin-associated microRNAs.

The effect of a protein on PR cell survival can be tested by incubating in vitro retinal tissue with the protein, or by expressing the protein in in vitro retinal tissue using the pRCVRN-test gene-IRES-ECFP construct.

### Pharmaceutical Compositions

The 3D retinal organoids of the present disclosure may be administered to a subject in need of therapy per se. Alternatively, the 3D retinal organoids of the present disclosure may be administered to a subject in need of therapy in a pharmaceutical composition mixed with a suitable carrier and/or using a delivery system.

As used herein, the term “pharmaceutical composition” refers to a preparation comprising a therapeutic agent or therapeutic agents in combination with other components, such as physiologically suitable carriers and excipients. The purpose of a pharmaceutical composition may be, e.g., to facilitate administration of a therapeutic agent to a subject and/or to facilitate persistence of the agent subsequent to administration.

As used herein, the term “therapeutic agent” may refer to either the 3D retinal tissue of the present disclosure or to a specific cell type or a combination of cell types within the 3D retinal tissue accountable for a biological effect in the subject.

As used herein, the terms “carrier” “physiologically acceptable carrier” and “biologically acceptable carrier” may be used interchangeably and refer to a diluent or a carrier substance that does not cause significant adverse effects or irritation in the subject and does not abrogate the biological activity or effect of the therapeutic agent. The term “excipient” refers to an inert substance added to a pharmaceutical composition to further facilitate administration of the therapeutic agent.

The therapeutic agents of the present disclosure may be administered as a component of a hydrogel, such as those described in US Patent Application Publication No. 2014/0341842, (Nov. 20, 2014), and U.S. Pat. Nos. 8,324,184 and 7,928,069.

The therapeutic agents of the present disclosure can also be administered in combination with other active ingredients, such as, for example, adjuvants, protease inhibitors, or other compatible drugs or compounds where such combination is seen to be desirable or advantageous in achieving the desired effects of the methods described herein.

### Kits

Also included in the present invention are kits. Such kits can include an agent or composition described herein and, in certain embodiments, instructions for administration. For example, a kit can comprise pluripotent cells (such as, for example, hESCs), culture media, and growth factors useful for steering the differentiation of the hESCs into 3D retinal organoids. Thus, in certain embodiments, a kit can comprise hESCs, Neurobasal® medium, Neurobasal®-A medium, noggin, bFGF, Dkk-1, IGF-1 and FGF-9. Such kits can be used to obtain the 3D retinal organoids of the invention or to facilitate performance of the methods described herein.

### EXAMPLES

The following examples are not intended to limit the scope of what the inventors regard as their invention nor are they intended to represent that the experiments below are all or the only experiments performed.

### Example 1: Generation of hESC-Derived In Vitro Retinal Tissue/3D Retinal Organoids

Composition of Neurobasal® Complete Medium.

1×N2, 1×B27 without retinoic acid, 1.1-glutamine (1%), 1% Minimal Essential Medium nonessential amino acid solution (MEM), 1. amphotericin-B/gentamicin (Life Technologies), BSA fraction V (0.1%) (Sigma-Aldrich), b-mercaptoethanol (0.1 mM; Sigma-Aldrich), and 94.8% (volume/volume) of Neurobasal® medium.

The derivation and maturation of hESC-derived 3D human retinal tissue has been recently described. Singh, R. K., et al., Characterization of Three-Dimensional Retinal Tissue Derived from Human Embryonic Stem Cells in Adherent Monolayer Cultures. Stem Cells Dev, 2015. 24(23): p. 2778-95, incorporated herein by reference in its entirety. Briefly, hESC (WA01, formerly H1) colonies were grown to 75-80% density in hESC medium (containing basic fibroblast growth factor (bFGF)). Medium was then replaced (Day 0) with hESC medium/Neurobasal® complete (NB) medium (1:1 ratio) with no bFGF and 100 ng/mL noggin morphogen (Sigma-Aldrich). On day 3, the medium was again replaced with 100% NB containing 1×N2, 1×B27, and 100 ng/mL noggin, and cultured for another 3 days. The recipe is described (Nasonkin et al. (2009) Long-term, stable differentiation of human embryonic stem cell-derived neural precursors grafted into the adult mammalian neostriatum. Stem Cells 27:2414-2426), except for the replacement of 1× Pen-Strep with 1×.amphotericin-B, 1× gentamicin. Thereafter, one-half of the conditioned medium was replaced every third day with fresh NB/N2/B27/noggin. At +2 weeks after initiating the protocol (i.e., 14 days after introduction of noggin to the culture), bFGF (Sigma-Aldrich) was added to cultures at a concentration of 10 ng/mL (retaining noggin at 100 ng/ml). At +4 weeks, retinal induction was induced by addition of DKK-1 and IGF-1 (both at 10 ng/mL; obtained from Sigma-Aldrich) to the noggin- and bFGF-containing cultures. After one week, in retinal induction medium, the induced retinal cells were transferred to Neurobasal® complete medium (recipe below) containing noggin (100 ng/mL), bFGF (10 ng/mL), and FGF9 (10 ng/mL) to promote neural retinal differentiation. Retinal organoids were maintained in Noggin, bFGF, FGF-9 containing medium for up to 12 weeks or more.

In addition, over the course of culture, the composition of Neurobasal® medium in Neurobasal® complete was very gradually changed weekly. Two types of Neurobasal® media (both from Life Technologies) were used: standard Neurobasal® (more suitable for culture of embryonic neural tissue) and Neurobasal®-A (NB-A), formulated for long-term culture of postnatal and adult neurons. The percentage (volume/volume) of NB-A in the culture medium was gradually increased from 2% at day 7 to 60% at 6-12 weeks to promote the survival of already differentiated postmitotic neurons while maintaining the differentiating progenitors. Thus, the composition of Neurobasal medium during culture was as follows: Days 0-7: 100% NB, no NB-A; days 8-14: 98% NB/2% NB-A; days 15-21: 93% NB/7% NB-A; days 21-28: 85% NB/15% NB-A; days 29-35: 70% NB/30% NB-A; and days 36+: 40% NB/60% NB-A. NB-A is expected to promote the survival of mature retinal neurons. About 50% of the medium was renewed every 3 days with fresh Neurobasal complete supplemented with noggin, bFGF, and FGF-9.

Three-dimensional hESC-derived retinal tissue aggregates (organoids) began to appear by about week 4 after initiation of the differentiation protocol, and rapidly increased in size by 6 weeks. The 3D growth of retina-like tissue aggregates in cultures was not synchronous, producing various shapes and sizes, and the number of such aggregates varied between 2-3 and 15 or more per 35-mm plate.

Maintaining hESC-derived retinal tissue on the plates at later time points (beyond 10-12 weeks) was accomplished by adding additional substrate (e.g., Matrigel®) to the cultures. The hESC-derived retinal tissue was characterized by quantitative reverse transcription-coupled polymerase chain reaction, immunoblot, immunohistochemistry (IHC), and electrophysiology at 6 weeks See Example 2.

### Example 2: Characterization of hESC-Derived In Vitro Retinal Tissue/3D Retinal Organoids

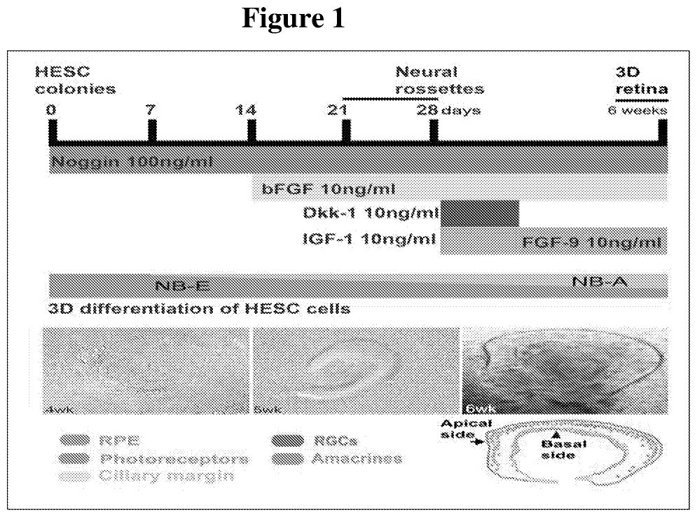

Robust and reproducible derivation of hESC-3D immature retinal tissue occurred in 6-8 weeks, with retinal cells growing out of the monolayer of hESC-derived neural cells further induced with a retinal induction protocol. See Example 1 and Singh, R. K., et al., Characterization of Three-Dimensional Retinal Tissue Derived from Human Embryonic Stem Cells in Adherent Monolayer Cultures. Stem Cells Dev, 2015. 24(23): p. 2778-95; Hambright, D., et al., Long-term survival and differentiation of retinal neurons derived from human embryonic stem cell lines in un-immunosuppressed mouse retina. Mol Vis, 2012. 18: p. 920-36. (FIG. 1). 3D retinal tissue comprised of all three retinal layers (ganglion cells, inner retinal neurons, photoreceptors) and retinal pigmented epithelium (RPE) is observed within 6-8 weeks after initiation of culture. Further maturation of this tissue (as manifested by short outer segment elongation, synaptogenesis and axonal elongation from ganglion cells) takes up to 3-4 months and is continuing as hESC-3D retinal tissue grows and matures in a dish.

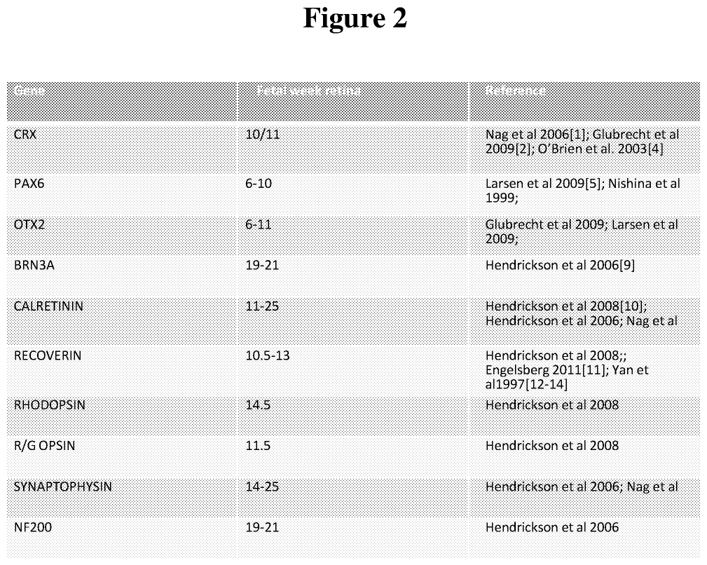

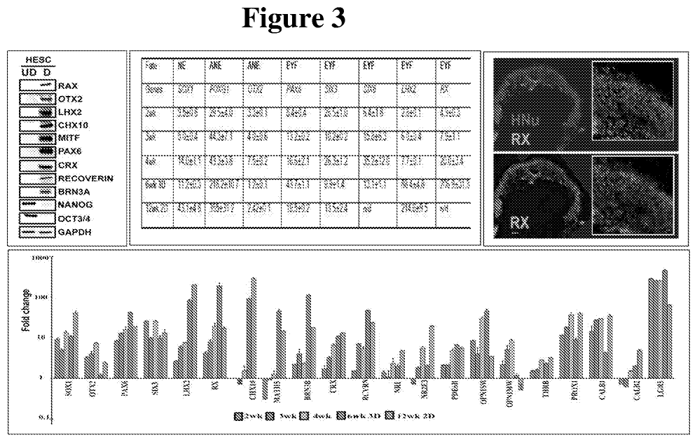

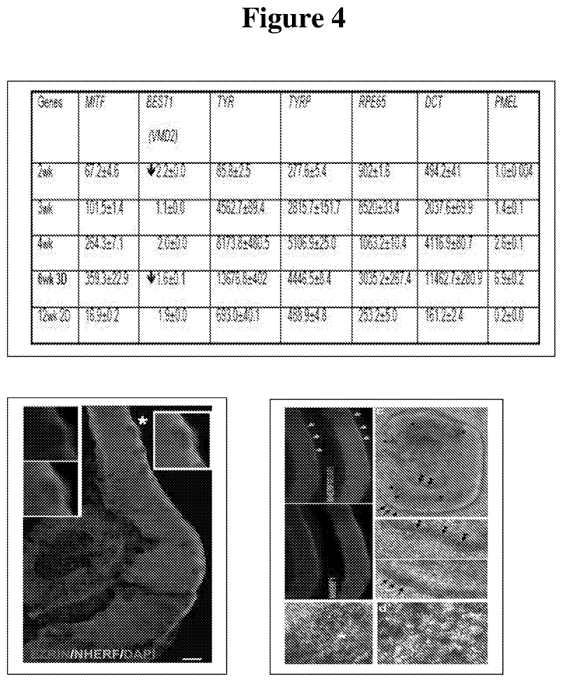

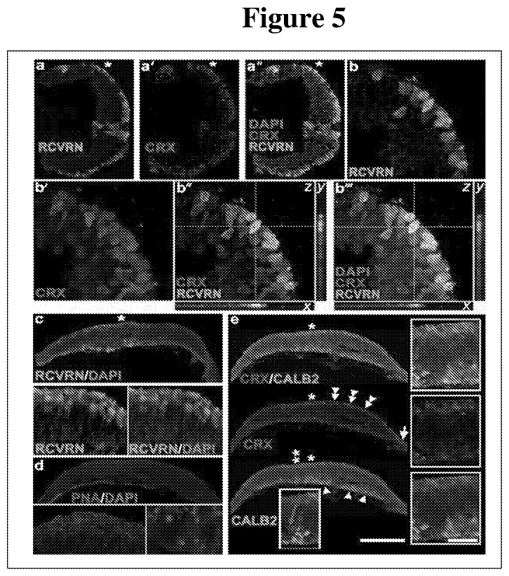

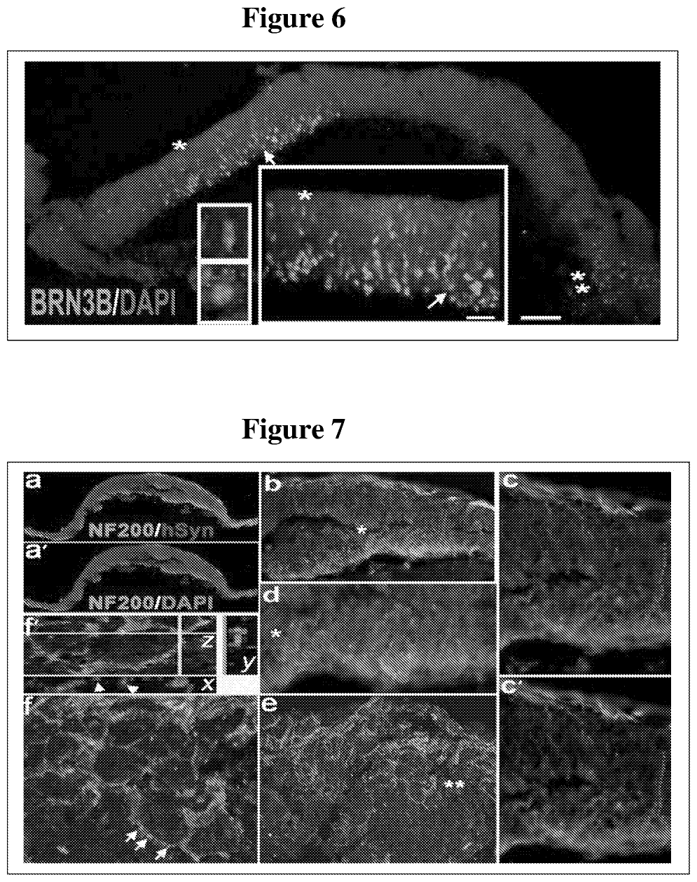

Reproducible recapitulation of mammalian retinogenesis was observed in growing hESC-3D retinal tissue, and was similar to that described in mouse retina, with close similarity between 8-week-old hESC-3D in vitro retinal tissue and human embryonic tissue of age 6-10 weeks, with respect to structure and timing of activation of markers CRX, PAX6, OTX2, BRN3A/B, CALRETININ (CALB2), RCVRN and RHO (determined by qRT-PCR and immunohistochemistry, IHC) (FIG. 2). Specifically, robust upregulation of all retinal field markers (LHX2, PAX6, RX, SIX3, SIX6) was observed in developing hESC-3D retinal tissue between 4-5 weeks by immunoblot, qRT-PCR and IHC (FIG. 3 top panel, left, middle and right panels, respectively). Furthermore, both markers of neural retina (FIG. 3, bottom panel above) and RPE (FIG. 4) were robustly expressed in hESC-3D retinal tissue. Abundant presence of PRs was observed in the basal side next to the RPE layer (FIG. 5) and developing retinal ganglion cells (RGCs) were also detected (FIG. 6) in 6-8 week old hESC-3D in vitro retinal tissue. Finally, robust synaptogenesis and axonogenesis occurred in hESC-3D retinal tissue (FIG. 7). Synaptogenesis began at around 6-8 weeks in some retinal organoids and continued and became more pronounced during the third and fourth month of hESC-3D retinal tissue development.

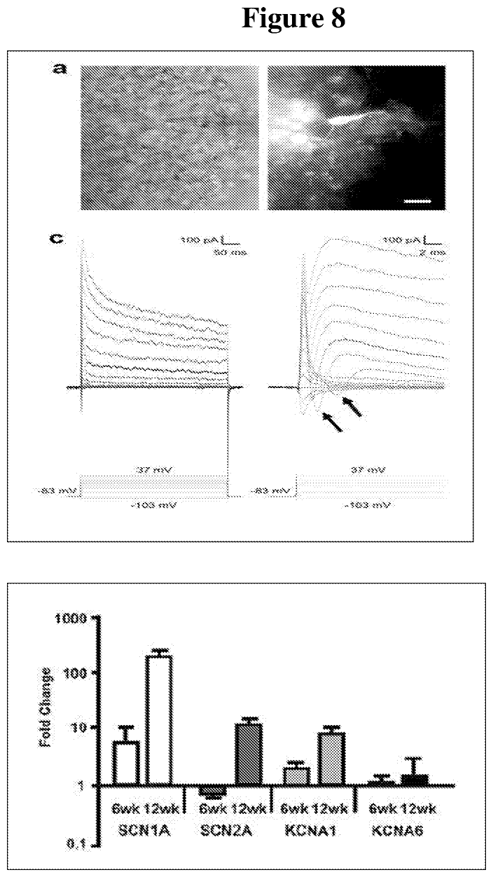

FIGS. 1-7 demonstrate that: 1) the hESC-derived 3D retinal organoids of the present disclosure have the organization of human retinal tissue, with a layer of RPE, PRs (with short outer segments), second order neurons with developed axons, and retinal ganglion cells with elongating axons; and 2) the hESC-derived 3D retinal organoids of the present disclosure also display robust synaptogenesis, which is most prominent in the apical and basal sides of the developing hESC-3D retinal tissue. It has also been observed that increased synaptogenesis coincides with increase in electrical activity within hESC-3D retinal tissue. While only some neurons showed Na⁺ and K⁺ currents in 6-8 week-old hESC-3D retinal tissue, almost all retinal neurons that were tested in 12-15-week-old hESC-3D retinal tissue aggregates displayed robust Na⁺ and K⁺ currents (FIG. 8).

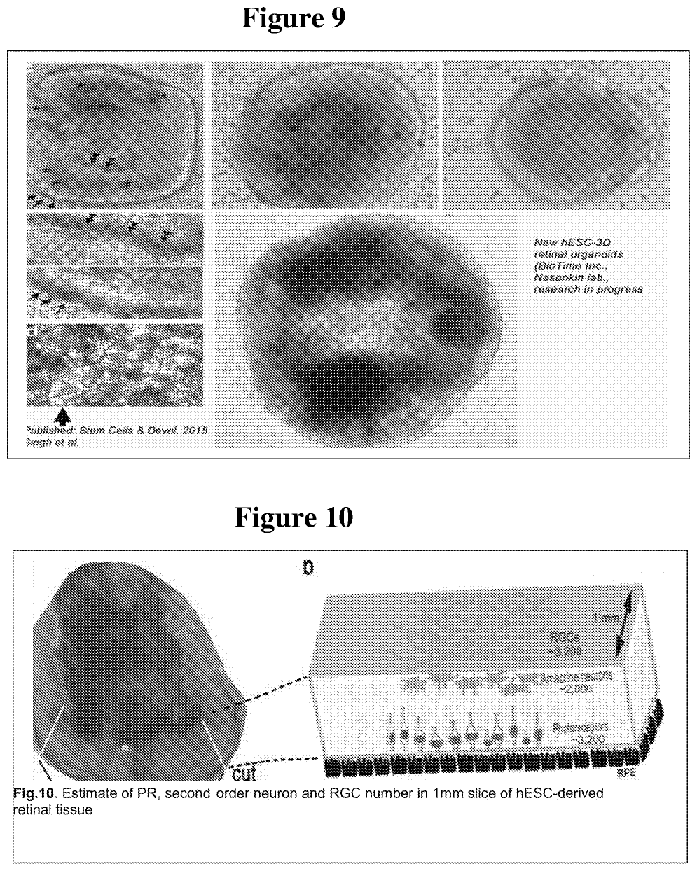

Collectively, the data in FIGS. 1-8 demonstrate that the hESC-derived 3D retinal organoids of the present disclosure represent a human retinal model which can survive in culture for several months, develop all retinal layers (RPE, PRs, inner retinal neurons and RGCs), displays robust synaptogenesis (especially in the apical (RGC) and inner retinal neuron layer, i.e., the PR-2nd order neuron junction), and exhibits robust electrical activity from about 2.5 to 3 months after development. Using the methods and compositions disclosed herein, it is possible to generate hundreds of such organoids. Exemplary organoids are shown in FIG. 9.

It is estimated that an average hESC-3D retinal tissue aggregate is 150-300 somas in diameter and 8-12 somas in thickness (which includes PRs, 2nd order neurons and RGCs) plus a RPE layer. It is also estimated that a typical hESC-3D retinal tissue aggregate generated as disclosed herein contains approximately 3,200 PRs, 2,000 amacrine neurons and 3,200 RGCs in one hESC-3D retinal tissue slice (FIG. 10). Collectively, these numbers allow a projection that several hESC-3D retinal tissue aggregates placed in one well of a 96-well plate are sufficient to evaluate the impact of gene overexpression or suppression (e.g., via siRNA), or a drug, on PR connectivity (i.e., synaptogenesis, synaptic activity) or/and regeneration (e.g., proliferation), creating an opportunity for rapid evaluation of the impact of many different factors on PR connectivity and/or regeneration simultaneously in a multi-well plate (i.e., a discovery-based approach).

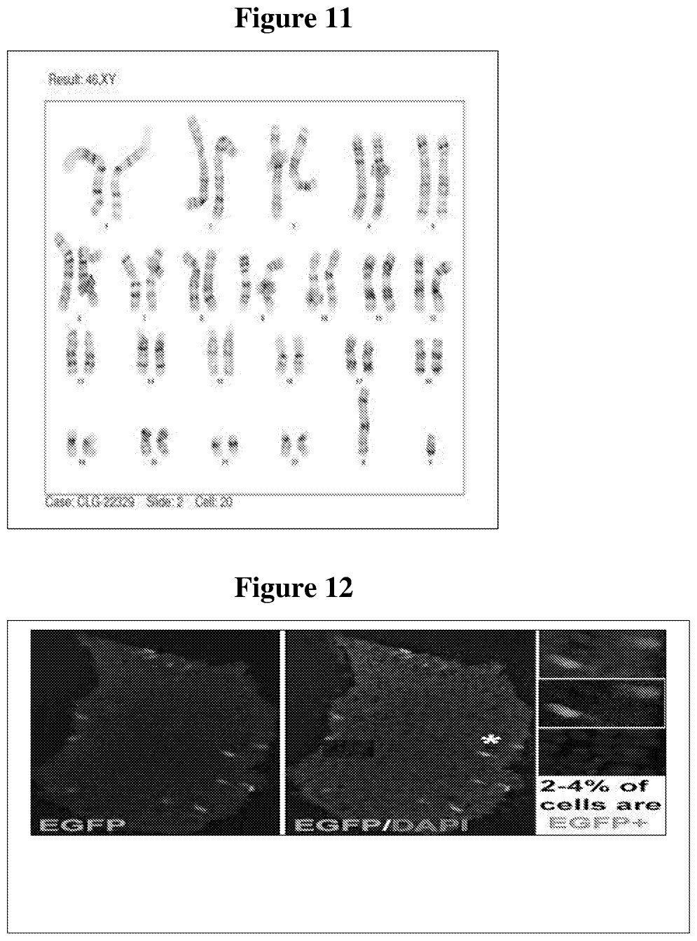

The hESC line H1 (WA01) used for derivation of 3D retinal tissue has a normal karyotype (46, X,Y) (FIG. 11), supporting the use of this hESC line for the derivation of 3D retinal organoids. The hESCs were successfully transfected with the plasmid EGFP-N1 (as a control to evaluate transfection efficiency) using FuGene 6 (FIG. 12). The same transfection protocol can also be used to isolate and subclone transgene-positive hESCs when using the CRISPR-Cas9 method (Ran, F. A., et al., Genome engineering using the CRISPR-Cas9 system. Nat Protoc, 2013. 8(11): p. 2281-308) to genetically modify the hESC-derived 3D retinal organoids of the present disclosure, (e.g., to engineer a mutation in the PDE6B gene in hESCs to create an Rd10-like RD phenotype in hESC-3D retinal tissue, see Example 6) or for routine stable transfection of hESCs (Gerrard, L., et al., Stably transfected human embryonic stem cell clones express OCT4-specific green fluorescent protein and maintain self-renewal and pluripotency. Stem Cells, 2005. 23(1): p. 124-33) and drug selection (Trion, S., et al., Identification and targeting of the ROSA26 locus in human embryonic stem cells. Nat Biotechnol, 2007. 25(12): p. 1477-82).

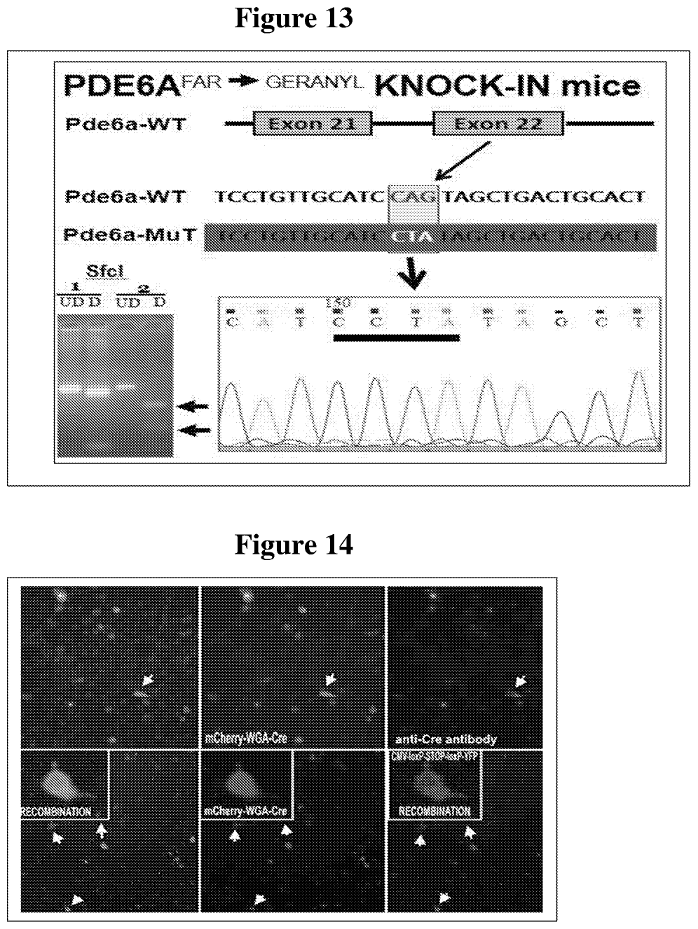

In certain embodiments, genetically modified hESC-derived 3D retinal organoids are obtained by using CRISPR-Cas9 genome engineering in their ES cell progenitors (Ran, F. A., et al., Genome engineering using the CRISPR-Cas9 system. Nat Protoc, 2013. 8(11): p. 2281-308). For example, the CRISPR-Cas9 system is used to engineer PDE6B mutation in hESCs (mimicking the Rd10 mouse mutation in Pde6brd10 (Chang, B., et al., Two mouse retinal degenerations caused by missense mutations in the beta-subunit of rod cGMP phosphodiesterase gene. Vision Res, 2007. 47(5): p. 624-33; Gargini, C., et al., Retinal organization in the retinal degeneration 10 (rd10) mutant mouse: a morphological and ERG study. J Comp Neurol, 2007. 500(2): p. 222-38). FIG. 13 shows experimental data from the generation of a 2 base pair change in the PDE6A gene in mouse ES cells by CRISPR-Cas9 engineering, according to a protocol by Ran et al. supra. The off-target mutation rate was reduced in this case by using a D10A (“single nickase) mutant version of Cas9 (pSpCas9n(BB)-2A-Puro) (Shen, B., et al., Efficient genome modification by CRISPR-Cas9 nickase with minimal off-target effects. Nat Methods, 2014. 11(4): p. 399-402).

Young PRs can be enriched from hESC-3D retinal tissue, for example, by CD73 sorting using FACS. See, for example, Postel, K., et al., Analysis of cell surface markers specific for transplantable rod photoreceptors. Mol Vis, 2013. 19: p. 2058-67; Lakowski, J., et al., Effective transplantation of photoreceptor precursor cells selected via cell surface antigen expression. Stem Cells, 2011. 29(9): p. 1391-404; Eberle, D., et al., Increased integration of transplanted CD73-positive photoreceptor precursors into adult mouse retina. Invest Ophthalmol Vis Sci, 2011. 52(9): p. 6462-71; and Koso, H., et al., CD73, a novel cell surface antigen that characterizes retinal photoreceptor precursor cells. Invest Ophthalmol Vis Sci, 2009. 50(11): p. 5411-8.

### Example 3: High Throughput Screening of PR Synaptic Connectivity and Regeneration Pathways Using hESC-Derived In Vitro Retinal Tissue/3D Retinal Organoids

This example describes the generation of a 3D human retinal tissue (organoid) culturing system for use in assaying for substances (e.g., genes, gene products, small organic molecules) which influence processes involved in retinal growth and development; for example, synaptogenesis, photoreceptor cell proliferation, etc. This assay system can be: (i) rapidly modified to predictably express new transgenes in PRs using the Tet-ON approach, (ii) maintained in 96 well plates for prolonged time, up to 24 weeks and longer, (iii) screened noninvasively in 96 well plates or other high throughput culturing systems to detect increase in synaptogenesis and PR regeneration, (iv) screened in 96 well plates or other high throughput culturing systems for small molecule drugs or biologics promoting PR survival; and (v) perfected to grow for up to 9 months and produce elongated PR outer segments.

A mCherry-IRES-WGA-Cre plasmid (Xu et al. (2013) Science 339(6125):1290-1295) was used to engineer a WGA-EGFP transsynaptic monosynaptic tracer fusion protein to label PR synaptic partners in hESC-3D retinal tissue. The mCherry-IRES-WGA-Cre plasmid has been validated by (i) transfecting the plasmid into HEK293 cells, and observing co-localization of mCherry and Cre (FIG. 14, upper three panels) and (ii) confirming Cre activity by co-transfecting the mCherry-IRES-WGA-Cre plasmid into HEK293 cells with a CMV-loxp-STOP-loxP-YFP plasmid that conditionally expresses the yellow fluorescent protein (YFP) reporter, and observing activation of YFP (FIG. 14, lower three panels). The integrity of the plasmid was further confirmed by DNA sequencing.

The human 3D retinal organoids described in Examples 1 and 2 are used in an assay for synaptic connectivity (synaptogenesis) in conjunction with the monosynaptic transsynaptic reporter construct pRCVRN-mCherry-IRES-(WGA˜EGFP). This reporter construct contains, in the following order, a recoverin (RCVN) promoter, sequences encoding a mCherry fluorophore, an internal ribosome entry site (IRES) or a self-cleaving 2A peptide from porcine teschovirus-1 (P2A) site (Kim et al., High Cleavage Efficiency of a 2A Peptide Derived from Porcine Teschovirus-1 in Human Cell Lines, Zebrafish and Mice. PLoS ONE, 2011, Vol. 6 (4): e18556) for bicistronic exression, and sequences encoding a wheat germ agglutinin (WGA)/enhanced green fluorescent protein (EGFP) fusion gene. The reporter construct is expressed in the cells of the organoids (e.g., by transfection), and the entire transcriptome of the reporter-expressing cells is evaluated by RNA-Seq to identify PR and synaptic connectivity-related genes/pathways activated or downregulated in the retinal organoids. Changes in gene expression, as detected by transcriptome analysis, are correlated with synaptic connectivity, as evidenced by expression of mCherry-negative, EGFP-positive cells, to identify genes and pathways involved in synaptogenesis.

Organoid cells can also optionally contain a tetracycline-inducible (Tet-ON) Flp-In transgene comprising a recoverin promoter, a flippase recognition target (Frt), an IRES and sequences encoding enhanced cyan fluorescent protein (ECFP).

Using, for example, transduction with lentiviral vectors; CRISPR-Cas9-mediated gene insertion or other methods known in the art (e.g., TALENs, ZFNs); hESCs expressing a monosynaptic transsynaptic reporter construct pRCVRN-mCherry-IRES-(WGA˜EGFP) and a Tetracycline-inducible (Tet-ON) Flp-In system vector (pRCVRN-Frt-IRES-ECFP) are generated. The hESCs are converted to 3D retinal organoids as described in Example 1, and the entire transcriptome of the organoids is evaluated at 8, 16 and 24 weeks by RNA-Seq to identify PR and synaptic connectivity-related genes/pathways activated in the-3D retinal organoid tissue. Voltage-sensitive dyes (Leao, R. N., et al., A voltage-sensitive dye-based assay for the identification of differentiated neurons derived from embryonic neural stem cell cultures. PLoS One, 2010. 5(11): p. e13833; Adams, D. S. and M. Levin, General principles for measuring resting membrane potential and ion concentration using fluorescent bioelectricity reporters. Cold Spring Harb Protoc, 2012. 2012(4): p. 385-97) and Ca2+-sensitive dyes are used to noninvasively monitor increase of synaptic maturation in organoid tissue, and presence of the WGA˜EGFP fusion protein is used to identify non-PR (EGFP⁺, mCherry⁻) retinal neurons synapsing on PRs (mCherry⁺, EGFP⁺). The number of such synaptic events in hESC-3D retina at 8, 16, and 24 weeks is measured.

Candidate genes to be tested for their effect on synaptogenesis are introduced into PR cells by inserting sequences encoding a gene of interest, or a fragment thereof, at the Frt site of the pRCVRN-Frt-IRES-ECFP construct, using FLP-mediated recombination. The pRCVRN-test gene-IRES-ECFP construct is introduced into pluripotent cells (also optionally containing the pRCVRN-mCherry-IRES-(WGA˜EGFP construct) and the pluripotent cells are converted to in vitro retinal tissue using the methods disclosed herein. Expression of the candidate gene is activated in organoid cultures using the tet-ON system (e.g., by adding doxycycline to the culture) and the effect on synaptogenesis is determined using methods described herein (e.g., appearance of EGFP⁺/mCherry⁻ cells, voltage sensitive dyes, electrophysiology etc.). In an exemplary method, the pRCVRN-mCherry-IRES-(WGA˜EGFP) and Tetracycline-inducible (Tet-ON) pRCVRN-Frt-IRES-ECFP reporters are introduced (via, e.g., lentiviral transgenes) into hESCs under conditions in which individual hESCs receive both transgenes (or conditions which select for such). Ten hESC clones having normal karyotype and carrying both transgenes are selected, frozen stocks of these clones are established, and expression of mCherry, EGFP, and ECFP is evaluated in developing PRs in hESC-3D retinal tissue. Clones in which activation of mCherry, EGFP and ECFP is restricted to PRs in hESC-3D retinal tissue are selected. Selection criteria include immunohistochemistry with anti-RCVRN Ab/mCherry/EGFP/ECFP, and anti-CRX Ab/mCherry/EGFP/ECFP using far-red fluorophore Alexa 647 for RCVRN or CRX, and observation of the pattern of mCherry[+], EGFP/ECFP[+] cell distribution. If necessary, flow cytometry and sorting for CD73⁺ cells (a PR marker) is conducted. PR cell bodies form a layer of cells primarily adjacent to the RPE layer. Singh, R. K., et al., Characterization of Three-Dimensional Retinal Tissue Derived from Human Embryonic Stem Cells in Adherent Monolayer Cultures. Stem Cells Dev, 2015. 24(23): p. 2778-95. Alternatively, CRISPR-Cas9 engineering (via a bicistronic system ˜IRES-mCherry, ˜IRES-WGA˜EGFP) is used, instead of lentiviral transgenes, to express mCherry and the WGA˜EGFP transsynaptic tracer in PRs.

To test this system, one of the ten clones described in the preceding paragraph is selected, and a pilot transgene (BDNF cDNA) is introduced at the site of the Frt sequences using the Flp-in system. Lu, H., et al., A rapid Flp-In system for expression of secreted H5N1 influenza hemagglutinin vaccine immunogen in mammalian cells. PLoS One, 2011. 6(2): p. e17297. hESC-3D retinal tissue is derived according to the method of Example 1, and BDNF expression is induced, e.g., with doxycycline (DOX). The synaptic connectivity of PRs to other retinal neurons in hESC-3D retinal tissue is then evaluated with or without BDNF transgene expression in PRs (e.g., in the presence or absence of DOX, respectively). Synaptogenesis between PR cells and second order retinal neurons, if it occurs, is observed in approximately 10-12 week old hESC-3D retinal tissue [Singh, R. K., et al., Characterization of Three-Dimensional Retinal Tissue Derived from Human Embryonic Stem Cells in Adherent Monolayer Cultures. Stem Cells Dev, 2015. 24(23): p. 2778-95]. An indication of synaptogenesis is migration of WGA˜EGFP transsynaptic monosynaptic tracer fusion protein from PRs into PR synaptic partners. Xu, W. and T. C. Sudhof, A neural circuit for memory specificity and generalization. Science, 2013. 339(6125): p. 1290-5; Braz, J. M., B. Rico, and A. I. Basbaum, Transneuronal tracing of diverse CNS circuits by Cre-mediated induction of wheat germ agglutinin in transgenic mice. Proc Natl Acad Sci USA, 2002. 99(23): p. 15148-53.

The reproducibility of these data from hESC-3D retinal tissue aggregates is further evaluated in a 96-well plate by measuring the activity of voltage-sensitive dyes (Adams, D. S. and M. Levin, Measuring resting membrane potential using the fluorescent voltage reporters DiBAC4(3) and CC2-DMPE. Cold Spring Harb Protoc, 2012. 2012(4): p. 459-64; Leao, R. N., et al., A voltage-sensitive dye-based assay for the identification of differentiated neurons derived from embryonic neural stem cell cultures. PLoS One, 2010. 5(11): p. e13833; Adams, D. S. and M. Levin, General principles for measuring resting membrane potential and ion concentration using fluorescent bioelectricity reporters. Cold Spring Harb Protoc, 2012. 2012(4): p. 385-97) and by measuring levels of EGFP in each well at 8, 16 and 24 weeks.

These data are correlated with electrophysiological measurements of hESC-3D retinal tissue in selected plates (Singh, R. K., et al., Characterization of Three-Dimensional Retinal Tissue Derived from Human Embryonic Stem Cells in Adherent Monolayer Cultures. Stem Cells Dev, 2015. 24(23): p. 2778-95), also with qRT-PCR data for expression of the SCN1A, SCN2A, KCNA1, KCNA6 genes; and with IHC data from selected hESC-3D retinal tissue aggregates (by counting the number of mCherry-negative/EGFP-positive neurons, which are not PRs but are PR synaptic partners). Selected hESC-3D retinal organoids are dissociated, and sorting by flow cytometry is conducted to evaluate the number of mCherry⁻/EGFP⁺ neurons, which are PR synaptic partners. In addition, four sets of BDNF-transgene-negative (i.e., “wild-type”) organoids are collected (from selected wells of a 96-well plate with comparable high activity of voltage-sensitive dyes) at 8, 16 and 24 weeks (total of 12 sets) for whole transcriptome analysis to determine if the development of hESC-3D retinal tissue aggregates is comparable in different wells. Evaluation of synaptic maturation in developing hESC-3D retinal tissue using Ca²⁺-sensitive and voltage-sensitive dyes (Adams, D. S. and M. Levin, Measuring resting membrane potential using the fluorescent voltage reporters DiBAC4(3) and CC2-DMPE. Cold Spring Harb Protoc, 2012. 2012(4): p. 459-64; Leao, R. N., et al., A voltage-sensitive dye-based assay for the identification of differentiated neurons derived from embryonic neural stem cell cultures. PLoS One, 2010. 5(11): p. e13833) is also conducted.

To maintain and mature hESC-3D retinal tissue aggregates for prolonged periods of time (up to 9 months), and achieve PR outer segment elongation, suitable Hydrogel support systems (based on proprietary HyStem® hydrogel technologies from ESI Bio, a subsidiary of BioTime, Inc.) are utilized. Hydrogels containing various morphogens, mitogens and trophic factors are used to achieve robust survival, growth and development of hESC-3D retinal tissue aggregates, to perfect retinal organoid culture, and to mimic, as closely as possible, the developing human retina.

hESC Culture, Genetic Engineering and Analysis

WA01 (formerly called H1), an established and tested hESC line (Thomson, J. A., et al., Embryonic stem cell lines derived from human blastocysts. Science, 1998. 282(5391): p. 1145-7) is cultured in feeder-free serum-free conditions using the TeSR1 medium (Ludwig, T. E., et al., Derivation of human embryonic stem cells in defined conditions. Nat Biotechnol, 2006. 24(2): p. 185-7 and protocol, supplied from Stem Cell Technologies (www.stemcell.com), with the addition of 200 ng/ml heparin to maintain a higher level of pluripotency and reduce the rate of spontaneous differentiation in hESC culture.

The pRCVRN-mCherry-IRES-(WGA˜EGFP) reporter is constructed by replacing WGA-cre, in the pRCVN-mCherry-IRES-WGA-Cre construct, with WGA˜EGFP using routine genetic engineering methods including PCR. Stable Genetic modification of hESC H1 (WA01), by introduction of pRCVRN-mCherry-IRES-(WGA˜EGFP) and Tetracycline-inducible (Tet-ON) pRCVRN-Frt-IRES-ECFP, is accomplished using lentiviral vectors and/or CRISPR-Cas9 technology. For use of lentiviral vectors to introduce transgenes into retinal cells, see, for example, Campbell, L. J., J. J. Willoughby, and A. M. Jensen, Two types of Tet-On transgenic lines for doxycycline-inducible gene expression in zebrafish rod photoreceptors and a gateway-based tet-on toolkit. PLoS One, 2012. 7(12): p. e51270; Le, Y. Z., et al., Inducible expression of cre recombinase in the retinal pigmented epithelium. Invest Ophthalmol Vis Sci, 2008. 49(3): p. 1248-53; and Chang, M. A., et al., Tetracycline-inducible system for photoreceptor-specific gene expression. Invest Ophthalmol Vis Sci, 2000. 41(13): p. 4281-7. Lentiviral vectors can maintain high titers while carrying up to 7.5-8 kb of transgene (al Yacoub, N., et al., Optimized production and concentration of lentiviral vectors containing large inserts. J Gene Med, 2007. 9(7): p. 579-84; and Jakobsson, J. and C. Lundberg, Lentiviral vectors for use in the central nervous system. Mol Ther, 2006. 13(3): p. 484-93); which is greater than the estimated size of the pRCVRN-mCherry-IRES WGA˜EGFP reporter; which is calculated to be 3-3.5 kb pRCVRN+0.768 kb mCherry+0.35 kb IRES+0.558 kb WGA+0.879 EGFP (Xu and Sudhof, supra; Raikhel and Wilkins (1987) Proc. Natl. Acad. Sci. USA 84(19):6745-6749).

For hESC subcloning, single hESCs are grown in 10 μM Rho-kinase inhibitor (ROCK), 40-60 subclones are picked (with the expectation that approximately every fifth hESC subclone carrys a lentiviral insertion), and transgene-positive subclones are selected by PCR. The subclones are expanded and karyotyped, and subclones with a normal karyotype (46 chromosomes) are selected and tested for pluripotency as described (Singh, R. K., et al., supra). One or more of the engineered hESC clones are used for experiments as outlined herein.

As an alternative to lentiviral-mediated introduction of transgenes, the CRISPR-Cas9 approach can also be used for targeted genome engineering in cells, including hESCs. Zhang, F., Y. Wen, and X. Guo, CRISPR/Cas9 for genome editing: progress, implications and challenges. Hum Mol Genet, 2014. 23(R1): p. R40-R46. With this approach, the reporter constructs (pRCVRN-mCherry-IRES-(WGA˜EGFP) and Tetracyclin-inducible (Tet-ON) pRCVRN-Frt-IRES-ECFP) are placed into the ubiquitously expressed “safe harbor” locus ROSA26 (Trion, S., et al., Identification and targeting of the ROSA26 locus in human embryonic stem cells. Nat Biotechnol, 2007. 25(12): p. 1477-82), to achieve reliable expression from the pRCVRN promoter that is not affected by the (transgene) position effect. Yin, Z., et al., Position effect variegation and epigenetic modification of a transgene in a pig model. Genet Mol Res, 2012. 11(1): p. 355-69; Peach, C. and J. Velten, Transgene expression variability (position effect) of CAT and GUS reporter genes driven by linked divergent T-DNA promoters. Plant Mol Biol, 1991. 17(1): p. 49-60.

CRISPR-Cas9 engineering follows the protocol of Ran et al. Briefly, guide RNA specific to the human ROSA26 locus (Trion, S., et al., Identification and targeting of the ROSA26 locus in human embryonic stem cells. Nat Biotechnol, 2007. 25(12): p. 1477-82) is designed using the CRISPR design tool (http://tools.genome-engineering.org) and cloned into Cas9 expression vectors (pSpCas9(BB)-2A-GFP, PX458; pSpCas9(BB)-2A-Puro, PX459; and pSpCas9n(BB)-2A-Puro (PX462). To reduce the off-target mutation frequency in human cells (Fu, Y. et al., High-frequency off-target mutagenesis induced by CRISPR-Cas nucleases in human cells. Nat Biotechnol, 2013. 31(9): p. 822-6), a D10A (“single nickase”) mutant version of Cas9 (pSpCas9n(BB)-2A-Puro) is used. Shen, B., et al., Efficient genome modification by CRISPR-Cas9 nickase with minimal off-target effects. Nat Methods, 2014. 11(4): p. 399-402. DNA (“Southern”) blotting is used to confirm that the transgene is integrated at a single genomic locus.

The donor plasmid used for targeting contains ROSA26 5′ and 3′ targeting arms (500 base pairs each) for homology-directed repair. WA01 cells are co-transfected with Cas9 vector and linearized targeting DNA, plated as single cells with 10 μM ROCK (Watanabe, K., et al., A ROCK inhibitor permits survival of dissociated human embryonic stem cells. Nat Biotechnol, 2007. 25(6): p. 681-6), and selected using 0.4 μg/mL puromycin for 48 hr. Colonies are grown and expanded for ˜3 weeks, then analyzed for targeted insertion in ROSA26 locus.

For introduction of test genes into the (Tet-ON) pRCVRN-Frt-IRES-ECFP reporter construct, the Flp-in system (ThermoFisher) design and protocols are used. See, for example, https://www.thermofisher.com/us/home/references/protocols/proteins-expression-isolation-and-analysis/protein-expression-protocol/flp-in-system-for-generating-constitutive-expression-cell-lines.htm.

For activation of expression of test genes inserted into the pRCVRN-Frt-IRES-ECFP reporter, the Tet-On system (Clontech) is used. See, for example, http://www.clontech.com/US/Products/Inducible Systems/Tetracycline Inducible_Expression/Tet-On_3G; and Campbell, L. J., J. J. Willoughby, and A. M. Jensen, Two types of Tet-On transgenic lines for doxycycline-inducible gene expression in zebrafish rod photoreceptors and a gateway-based tet-on toolkit. PLoS One, 2012. 7(12): p. e51270.

For assays, hESC-3D retinal tissue aggregates are cultured in 96-well plates at a density of one aggregate per well. Density can be increased (e.g., to several aggregates per well) when the retinal tissue aggregates develop and mature at a similar pace in culture. Having several organoids per well will enable generation of flow-sorting, IHC, RNA-Seq and electrophysiology data from the same plate.

HyStem® hydrogel technologies (ESI Bio, a subsidiary of BioTime, Inc.) are used in certain cultures. One or more morphogens, mitogens, and/or trophic factors are embedded in the hydrogel to sustain growth and maturation of RPE and neural retina in hESC-3D retinal tissue. Exemplary morphogens include, but are not limited to Indian hedgehog homologue (IHH) and sonic hedgehog (SHH). Nasonkin, I. O., et al., Conditional knockdown of DNA methyltransferase 1 reveals a key role of retinal pigment epithelium integrity in photoreceptor outer segment morphogenesis. Development, 2013. 140(6): p. 1330-41.

Use of voltage-sensitive dyes is conducted according to instructions from Thermo Fisher Scientific on using voltage-sensitive dyes, Cat #k1016 and publications (Adams, D. S. and M. Levin, Measuring resting membrane potential using the fluorescent voltage reporters DiBAC4(3) and CC2-DMPE. Cold Spring Harb Protoc, 2012. 2012(4): p. 459-64; Leao, R. N., et al., A voltage-sensitive dye-based assay for the identification of differentiated neurons derived from embryonic neural stem cell cultures. PLoS One, 2010. 5(11): p. e13833). Alternatively, FURA2 (Thermo Fisher Scientific, Cat. #F1221) is used.

Electrophysiology recordings are conducted as described. Singh, R. K., et al., Characterization of Three-Dimensional Retinal Tissue Derived from Human Embryonic Stem Cells in Adherent Monolayer Cultures. Stem Cells Dev, 2015. 24(23): p. 2778-95]. Flow cytometry sorting is used to count the number of PRs [mCherry-positive, EGFP-positive neurons] and their synaptic partners [mCherry-negative, EGFP-positive cells]. The number of PRs [mCherry-positive, EGFP-positive neurons] and their synaptic partners [mCherry-negative, EGFP-positive] are evaluated by routine immunohistochemistry (IHC). Data from whole transcriptome analysis (RNA-Seq) is analyzed to identify PR- and synaptic connectivity-related genes and pathways that are activated or downregulated in the human retinal organoid model.

### Example 4: Screening for Optimal Combinations of Factors for Upregulating Synaptogenesis and Photoreceptor-Second Neuron Connectivity in Human Retina

In certain embodiments, assays utilizing in vitro retinal tissue (i.e., 3D retinal organoids) are used to define and optimize combinations of specific factors which significantly upregulate synaptogenesis in hESC-3D human retinal tissue (as monitored by voltage-sensitive dyes, Ca²⁺ dye, quantitative RT-PCR, localization of the monosynaptic trans synaptic tracer WGA-EGFP, electrophysiology and IHC); and to identify and optimize combinations of factors that enhance connectivity of PRs to 2nd order retinal neurons. Several sets of optimal conditions are selected; using the criteria of: (1) upregulated functional activity, (2) synaptogenesis and (3) connectivity of mCherry-positive, EGFP-positive PRs to mCherry-negative, WGA-EGFP-positive second-order retinal neurons. Whole transcriptome analysis of 3D retinal organoids is conducted under optimal conditions selected as described above to identify pathways (i.e., small molecule drug targets) involved in enhancement of PR-2nd order neuron synaptic connectivity.

High throughput screening of synaptogenesis in hESC-3D retinal tissue cultured in 96-wells (or other suitable culture vessels) as described supra enables rapid screening of dozens of transgenes (such as BDNF, CNTF) and/or chemicals (such as db cAMP, DHA, taurine) and/or inhibitors/agonists of synaptogenesis/axonal elongation and connectivity (e.g., activity-induced, light-induced, neurotransmitter-driven, channelrhodopsin-activated, voltage-gated channel-promoted agonists or antagonists). Exemplary agonists and/or antagonists reported to positively impact PR synaptic connectivity and axonogenesis are set forth in Table 1, below.

TABLE 1

| | | | | | | |
|---|---|---|---|---|---|---|
| DHA | Uridine | DA | Osteopontin | SynCAM1 | GAD65 | SNAP-25 |
| dbcAMP | Choline | L-Glutamate | Netrin | PCDH-gamma | mGluR6 | Syntaxin-1 |
| cGMP | Spadin | 5HT | SEMA-1 | THBS1 | D2 DopamineR | Piccolo |
| HDACinhib | Ketamin | GABA | bFGF | PSD95 | Wnt7A | RIBEYE |
| Taurine | NMDAmod | Glycine | N-Cadherin | SYN | BMP7 | Bassoon |
| Lithuim-Cl | Testosterone | AMPA | NCAM | β-Neurexin | SHH | CACNA1F |
| Ret. Acid | Estradiol | B/GDNF | Dscam | GABAAreceptor | ChR2 | SCN1A |
| ATP/ADP | ACh | NOS | Sidekick-1 | GlyR | Rhodopsin | Ca2+ATPase |
| Ritalin | NMDA | Oncomodulin | Neuroligin | VGLUT1 | V-ATPase | KCNA1 |

Data using this multiplex screening strategy is generated according to the methods described in Examples 2 and 3. Each substance listed in Table 1 is tested in quadruplicate, in 4 wells of a 96-well plate, with 4-20 hESC-3D retinal tissue aggregates tested for each substance. The best candidates are selected for screening various permutations of molecules/factors. A large number of permutations, each combining several promising molecules/factors that promote synaptogenesis and/or PR-2nd order neuron connectivity, are tested together.

### Example 5: Evaluation of Sustained Expression of Genes Implicated in Developmental Plasticity and Dedifferentiation on PR Regeneration Using hESC-3D Retinal Model

Three-dimensional retinal organoids (i.e., in vitro retinal tissue) are used in assays to detect substances (e.g., gene products) that stimulate proliferation of photoreceptor cells; for example, genes involved in developmental plasticity and dedifferentiation.

To this end, several DOX-inducible Tet-ON transgenes are tested in hESC-3D retinal tissue, alone and in combination with one another, for the ability of inducible and transient expression of these genes to induce changes in PR plasticity. Initially, individual genes and/or conditions are tested (in quadruplicate, 4 wells, 4-20 hESC-3D retinal tissue aggregates/each condition) and the best candidates are selected for screening in combination. The criteria for selection include increase in mitosis in the PR layer (next to the RPE layer), increase in PR numbers, increase in mCherry fluorescence and increase in EGFP fluorescence. Subsequently, combinations of successful genes and/or conditions identified in the first step are tested together, using the same criteria.

Transiently turning off tumor suppressor genes p53, ARF and RB as outlined earlier (Pajcini, K. V., et al., Transient inactivation of Rb and ARF yields regenerative cells from postmitotic mammalian muscle. Cell Stem Cell, 2010. 7(2): p. 198-213; Hesse, R. G., et al., The human ARF tumor suppressor senses blastema activity and suppresses epimorphic tissue regeneration. Elife, 2015. 4), in conjunction with transient activation of certain pluripotency/neural plasticity genes (e.g., KLF4, SALL4, OCT3/4, MYC, NGN2, ASCL1, MYOD1) or/and retinal field/PR progenitor genes (e.g., PAX6, RX, SIX3, SIX6, OTX2) by DOX induction enable some PRs to reenter mitosis. In addition, hESC-3D retinal tissue is incubated with exosome preparations from progenitor cells, since exosome preparations from progenitor cells reportedly possess regeneration properties (Quesenberry, P. J., et al., Cellular phenotype and extracellular vesicles: basic and clinical considerations. Stem Cells Dev, 2014. 23(13): p. 1429-36; Katsman, D., et al., Embryonic stem cell-derived microvesicles induce gene expression changes in Muller cells of the retina. PLoS One, 2012. 7(11): p. e50417; De Jong, O. G., et al., Extracellular vesicles: potential roles in regenerative medicine. Front Immunol, 2014. 5: p. 608; Takeda, Y. S. and Q. Xu, Synthetic and nature-derived lipid nanoparticles for neural regeneration. Neural Regen Res, 2015. 10(5): p. 689-90; Stevanato, L., et al., Investigation of Content, Stoichiometry and Transfer of miRNA from Human Neural Stem Cell Line Derived Exosomes. PLoS One, 2016. 11(1): p. e0146353).

For both transgene-based and exosome-based approaches for regeneration of PRs, mCherry and EGFP fluorescence are used as initial readouts to monitor PR regeneration noninvasively, followed by conducting Red-Green flow-sorting from papain-dissociated 3D retinal tissue, immunohistochemistry, counting PR cell number, and counting the number of dividing Ki67+ cells. hESC-3D retinal tissue phenotype is observed (e.g., by qRT-PCR and/or IHC) after DOX activation of siRNA targeted to p53 and/or ARF and/or RB; PR numbers are measured and PR connectivity is evaluated (as described in previous Examples). Inactivation of tumor suppressor gene(s) is then combined with DOX-induced expression of one or more plasticity genes and/or one or more retinal field genes; and PR numbers, mitotic activity and connectivity are evaluated again. Reduction of complexity is achieved by eliminating redundant genes to obtain a combination of gene activation and/or repression which will enable PRs to reenter mitosis, maintain PR cell fate (rather than initiate tumors) and connect to 2nd order neurons.

Methods are described in Examples 2-4. Exosomes are prepared by methods known in the art and previously disclosed, e.g., in U.S. patent application Ser. No. 14/748,215.

### Example 6: Retinal Organoid System to Assay for Factors that Promote Photoreceptor Cell Survival

This example describes the generation of a 3D retinal tissue culturing system for detection of substances that promote PR cell survival and/or prevent PR cell degeneration, which can be (i) rapidly modified to predictably express new transgenes in PRs using the Tet-ON approach, (ii) maintained in 96 well plates for prolonged time, up to 24-36 weeks and longer, and (iii) screened noninvasively in 96 well plates to detect increase in synaptogenesis and PR survival. Combining the hESC-3D retinal tissue model with rapid screening in 96-well plates allows identification of the most effective therapies for support of degenerating PRs. Such issues cannot be addressed through tissue culture methods (lack of complexity) or animal modeling (too slow, too costly, not human). hESC-3D retinal tissue provides a suitable biological niche for testing questions related to PR cell survival and activity, including the RPE-PR-2nd order retinal neuron niche in the basal side.

Introduction of PDE6B Mutation into hESCs

Genetic mutations in enzymes involved the cGMP-hydrolyzing enzyme PDE6 are seen in up to 10% of human RP cases, and are known to cause PR cell death. Such mutations form the basis for several different mouse models for RP, including rd1 and rd10. Sancho-Pelluz, J., et al., Photoreceptor cell death mechanisms in inherited retinal degeneration. Mol Neurobiol, 2008. 38(3): p. 253-69; Veleri, S., et al., Biology and therapy of inherited retinal degenerative disease: insights from mouse models. Dis Model Mech, 2015. 8(2): p. 109-29. Using the CRISPR-Cas9 system, a PDE6B mutation is introduced into hESCs; optionally expressing a monosynaptic transsynaptic reporter construct pRCVRN-mCherry-IRES-(WGA˜EGFP) and/or a Tetracycline-inducible (Tet-ON) Flp-In system (pRCVRN-Frt-IRES-ECFP) to generate a “mutant” line. The generation of hESCs containing the two reporter constructs (the “control” line) is described in Example 3.

Mutant and control hESCs are converted to in vitro retinal tissue (i.e., retinal organoids) using the procedure described in Example 1, and PR cell survival is assayed in the control and mutant lines at defined time periods (e.g., 8, 16, 24, 36 weeks) using IHC/histology. In addition, the whole transcriptomes of control and mutant organoids are compared (e.g., at 8, 16, 24, 36 weeks) by RNA-Seq. to identify PR and synaptic connectivity-related changes in mutant hESC-3D retinal tissue indicative of retinal degeneration (RD). Voltage-sensitive dyes and Ca²⁺-sensitive dyes are used to noninvasively monitor increase of synaptic maturation in hESC-3D retina, as a sign of the degree of PR-inner retinal neuron connectivity. The presence of the WGA˜EGFP fusion protein in the synaptic partners of (EGFP⁺, mCherry⁺) PRs is used as an additional sign of PR-inner retinal neuron connectivity. PR synaptic partners are expected to be mCherry⁻/EGFP⁺, if such synaptic connectivity is not destroyed by RD symptoms. The number of mCherry⁻/EGFP⁺ cells is quantified by IHC and a possible correlation between the number of PR synaptic partners and the EGFP fluorescence in 96-wells (measured noninvasively) is investigated. If a correlation is observed, it provides a simple, noninvasive method to evaluate preservation of PR-inner neuron synaptic connectivity in a 96-well format as a way to monitor PR degeneration/survival.

Separately, the luciferase gene is tested to determine if it provides a more reliable and/or sensitive reporter than mCherry or EGFP for noninvasively screening for PR survival and preservation of PR-inner retinal neuron connectivity.

Drug-Induced PR Degeneration Models

In addition to using organoids whose cells contain the PDE6B mutation as a model of PR degeneration; drug-treated organoids can also be used. For example, a DOX-inducible lentiviral transgene encoding ataxin-7(Q90) is integrated into the genome of hESCs used to make retinal organoids. In the organoids, ataxin-7(Q90) is overexpressed in rod cells (via the RCVRN promoter), causing severe rod cell degeneration after DOX induction.

A second drug-induced PR degeneration model relies on treatment of retinal organoids with N-methyl, N-nitrosourea (MNU), an alkylating agent, which causes selective and progressive PR cell death involving the caspase pathway, within 7 days after application.

Another method to induce PR degeneration is to modulate cGMP-dependent protein kinase (PKG) in PRs using the PKG agonist 8-pCPT-PETcGMP (Biolog, Inc.). Activation of cGMP-dependent protein kinase is a hallmark of photoreceptor degeneration in the mouse rd1 and rd2 PR degeneration models. When induced in wild-type retinas, PKG activity was both necessary and sufficient to trigger cGMP-mediated photoreceptor cell death. Paquet-Durand, F., et al., PKG activity causes photoreceptor cell death in two retinitis pigmentosa models. J. Neurochem, 2009. 108(3): p. 796-810.

The PDE5/6-specific inhibitor zaprinast (Sigma, Stockholm/Sweden) can also be used to induce PR degeneration. Paquet-Durand et al., supra. Treatment with zaprinast (100 μM) raises intracellular cGMP and induces PR degeneration at a level comparable to that observed in the mouse rd1 model. Vallazza-Deschamps, G., et al., Excessive activation of cyclic nucleotide-gated channels contributes to neuronal degeneration of photoreceptors. Eur J Neurosci, 2005. 22(5): p. 1013-22.

### Example 7: Screening for Factors (and Combinations of Factors) that Promote Photoreceptor Survival

PR neuroprotection mediated by trophic factors, epigenetic modulators and/or metabolic changes induced in PRs is a feasible, noninvasive and broadly applicable way to alleviate blindness caused by PR cell death. Providing long-lasting trophic support to PRs (Yu, D. and G. A. Silva, Stem cell sources and therapeutic approaches for central nervous system and neural retinal disorders. Neurosurg Focus, 2008. 24(3-4): p. E11; Ramsden, C. M., et al., Stem cells in retinal regeneration: past, present and future. Development, 2013. 140(12): p. 2576-85; Stern, J. and S. Temple, Stem cells for retinal repair. Dev Ophthalmol, 2014. 53: p. 70-80) shows promise in alleviating PR cell death and is being evaluated in clinical trials (McGill, T. J., et al., Transplantation of human central nervous system stem cells—neuroprotection in retinal degeneration. Eur J Neurosci, 2012. 35(3): p. 468-77).

To develop a retinal organoid-based model system for investigating the effects of trophic factors, mitogens, epigenetic modulators and metabolic alterations on RP cell survival, ten clones of hESCs carrying the pRCVRN-mCherry-IRES-(WGA˜EGFP) and Tetracycline-inducible (Tet-ON) pRCVRN-Frt-IRES-ECFP lentiviral transgenes (described in Example 3), having normal karyotype, are obtained and frozen stocks are established. Retinal organoids (i.e., hESC-3D in vitro retinal tissue) are derived from these ten hESC lines, and the expression of mCherry, EGFP, and ECFP in developing PRs in the organoids is assessed by IHC with anti-RCVRN Ab/mCherry/EGFP/ECFP fluorescence, and anti-CRX Ab/mCherry/EGFP/ECFP fluorescence using far-red fluorophore Alexa 647 for RCVRN or CRX Ab, observing the pattern of mCherry⁺, EGFP/ECFP⁺ cell distribution and, if necessary, conducting CD73 flow sorting of PRs to determine the number of cells that are mCherry⁺/EGFP/ECFP⁺. A single clone in which mCherry, EGFP, and ECFP activation are maximal, in which expression is restricted to PRs in hESC-3D retinal tissue, and in which ECFP expression is induced by DOX is selected.

The PDE6B mutation (identical to the mouse rd10 mutation) is then introduced into the selected clone by CRISPR-Cas9 engineering.

Evaluating RD in hESC-3D Retinal Tissue with PDE6B Mutation

Organoids (hESC-3D in vitro retinal tissue) are produced from “Control” and “Mutant” hESC clones, as described in the previous example. 96 control organoids and 96 mutant organoids are cultured at a density of one organoid/well of a 96-well plate. Organoids are exposed to test substances; and PR survival, PR degeneration and PR-2nd order neuron synaptic connectivity are evaluated at 8, 16, 24 and optionally 36 weeks, as described supra. For example, indicia of retinal degeneration are determined by IHC (for mCherry, EGFP, and using photoreceptor cell-specific antibodies) and measurement of the activity of voltage-sensitive dyes. These data are correlated with electrophysiological measurements of hESC-3D retinal tissue in selected plates (Singh, R. K., et al., Characterization of Three-Dimensional Retinal Tissue Derived from Human Embryonic Stem Cells in Adherent Monolayer Cultures. Stem Cells Dev, 2015. 24(23): p. 2778-95); with qRT-PCR data for SCN1A, SCN2A, KCNA1, KCNA6 (Singh et al. supra); with IHC data from selected hESC-3D retinal tissue aggregates (by counting the number of MCherry⁺ PRs, and mCherry⁻/EGFP⁺ neurons (which are not PRs); and with antibody detection of cleaved Caspase-3 (a marker of apoptosis). Optionally, selected hESC-3D retinal organoids are dissociated and flow cytometry is conducted to evaluate the number of mCherry⁺ PRs and mCherry⁻/EGFP⁺ neurons, which are PR synaptic partners. Finally, at each timepoint (8, 16, 24 and optionally 36 weeks), 4-6 organoids are collected from each of the “Control” and “Mutant” sets, and RNA-Seq is conducted to delineate RD-related changes in the transcriptome of “Mutant” organoids.

Similar measurements are conducted on control organoids (i.e., organoids whose cells have a wild-type PDE6B gene) treated with, for example, MNU, 8-pCPT-PETcGMP or zaprinast to induce PR cell degeneration.

Organoids Expressing Transgenes

Genes and/or cDNAs encoding trophic factors (TF) and/or mitogens (M) (e.g., (BDNF, GDNF, NGF, NT3, bFGF, CNTF and/or PEDF cDNA) are introduced into the (Tet-ON) pRCVRN-Frt-IRES-ECFP transgene in a PDE6B-mutant hESc line selected as described supra in this Example, using the Flp-in system (Lu, H., et al., A rapid Flp-In system for expression of secreted H5N1 influenza hemagglutinin vaccine immunogen in mammalian cells. PLoS One, 2011. 6(2): p. e17297) to introduce the gene or cDNA into the Frt site. “Mutant” organoids (i.e., organoids whose cells contain a PDE6B mutation) are then derived from these hESCs with an integrated TF or M transgene. Expression of the TF or M transgene is induced with DOX, and mutant organoids expressing the transgene are compared with mutant organoids that do not express the transgene. For example, PR proliferation and the synaptic connectivity of PRs to other retinal neurons is evaluated as described elsewhere herein. Measurements are conducted in 96-well plates containing organoid material, and reproducibility of the data is evaluated by measuring the activity of voltage-sensitive dyes in each individual organoid in 96-well plates, as well as EGFP and mCherry levels in every well at, for example, 8, 16 and 24 weeks. These data are correlated with electrophysiological measurements of hESC-3D retinal tissue in selected plates, with qRT-PCR data for SCN1A, SCN2A, KCNA1, KCNA6, and with IHC data from selected hESC-3D retinal tissue aggregates by counting the number of mCherry⁻/EGFP⁺ neurons, which are not PRs. Optionally, selected hESC-3D retinal organoids are dissociated and flow cytometric sorting is conducted to evaluate the number of mCherry⁺ PRs and mCherry⁻/EGFP⁺ neurons, which are PR synaptic partners. Organoids are collected for RNA-Seq experiments as well.

Once it is determined which trophic factors and/or mitogens provide neuroprotection, whole transcriptome analysis is conducted on 3 sets of transgene-negative and 3 sets of transgene-positive organoids with induced PR degeneration at 8 weeks (4 organoids), 16 weeks (4 organoids) and 24 weeks (4 organoids) to delineate neuroprotective changes induced by expression of selected trophic factors and mitogens. Ca²⁺-sensitive dyes are also used as a sensor of synaptic activity in developing hESC-3D retinal tissue.

Alternatively, rather than using integrated transgenes to provide mitogens and/or trophic factors, mitogens and/or trophic factors of choice can be included in the cell culture medium, for example, by adding a predetermined concentration of M/TF into the wells of 96-well plates every other day. In addition, small molecule organic compounds are tested for neuroprotection by addition to the culture medium.

Assays for Multiple Mitogens and/or Trophic Factors

If two or more mitogens and/or trophic factors are shown to prevent PR cell degradation, retinal organoids containing a plurality of mitogens/trophic factors are tested to determine optimal combinations of mitogens and/or trophic factors. For these experiments, a plurality of colonies of PDE6B-mutant hESCs, each containing a single different M or TF construct, are dispersed into single cells, and seeded at high density on Matrigel®, using equal number of hESCs of each type (e.g., 50% BDNF-containing hESCs+50% bFGF-containing hESCs, or 33% BDNF-containing hESCs+33% NGF-containing hESCs+33% CNTF-containing hESCs). Retinal organoids (i.e., hESC-3D in vitro retinal tissue) are derived from these mixed cultures according to the methods described in Example 1; the organoids will thus contain approximately equal number of cells carrying each of the selected transgenes. Assays for PR cell neuroprotection, as described above, are conducted to identify the combination(s) of factors providing optimal prevention of PR cell degradation.

Provision of PR Cell Neuroprotection by Exosomes

Exosomes obtained from progenitor/stem cells reportedly possess neuroprotective properties, promoting neuronal survival and connectivity. They are reported to contain trophic factors and mitogens, as well as microRNAs with potent biological activities including neuroprotection and neural regeneration. Accordingly, exosomes prepared from proprietary hESC-derived progenitor lines (West, M. D., et al., The ACTCellerate initiative: large-scale combinatorial cloning of novel human embryonic stem cell derivatives. Regen Med, 2008. 3(3): 287-308) are tested as new vehicles for delivery of neuroprotective substances to degenerating PRs in in vitro retinal tissue as described herein.

For these experiments, retinal organoids derived from PDE6B-mutant hESCS as described herein, optionally containing the pRCVRN-mCherry-IRES-(WGA˜EGFP) transgene; are contacted with exosome preparations, and measurements of PR proliferation, PR survival and synaptic activity are conducted as described above. mCherry and EGFP are used as initial readouts to monitor PR regeneration noninvasively, followed by conducting Red-Green flow-sorting from papain-dissociated 3D retinal tissue, MC, and counts of PR number.

The exosome-based approach allows the identification of new molecules supporting PR survival by (i) identifying exosome preparations ameliorating PR cell death in the hESC-3D retinal tissue model and (ii) deciphering the exosome content within these preparations; e.g., by identification of microRNAs by routine microRNA preparation-sequencing, (Qiagen); and/or identification of proteins by, e.g., 2D proteome analysis.

Assay Criteria

To obtain statistically significant results, data (e.g., flow cytometry, IHC, voltage-sensitive dye activity, RNA-Seq, quantification of mCherry, EGFP fluorescence and Luciferase) are generated from multiple hESC-3D retinal tissue aggregates per each time point of organoid differentiation (8, 16, 24, and optionally 36 weeks). For RNA-Seq, four organoids per time point are selected, from different wells of a 96-well plate. Similar levels of voltage-sensitive dye activation are interpreted to indicate similar level of synaptogenesis within the tissue; providing correlations are established with voltage-sensitive dye activity (by live imaging), synaptogenesis (by IHC), electrophysiology and qRT-PCR (using voltage-gated channel genes as targets).

Transsynaptic tracing of PR synaptic partners is measured by migration of WGA-EGFP via synapses formed between (mCherry+, EGFP⁺) PRs and their synaptic partners, to highlight the neurons (mCherry⁻, EGFP⁺) in hESC-3D retinal tissue, which are synaptically connected to PRs. MC data is examined for connectivity between (mCherry⁺, EGFP⁺) PRs and (mCherry⁻, EGFP⁺ neurons (PR synaptic partners) prior to flow cytometry and counting (Red⁺, Green⁺) versus (Red⁻, Green⁺).

It is possible that transsynaptic migration of WGA-EGFP into PR synaptic partners may also be detected noninvasively because of increase in EGFP-positive cell numbers in hESC-3D retinal organoids. If true, an additional noninvasive readout method of monitoring synaptogenesis in hESC-3D retina is available.

RNA-Seq data (i.e., whole transcriptome analysis) is used to identify pathways and/or genes in human retina that are involved in neuroprotection. These pathways and/or genes constitute future drug targets.

### Example 8: Screens for Chromatin Modifying Factors that Promote Photoreceptor Survival

DNA methylation, histone methylation and histone acetylation are key epigenetic modifications that help govern heterochromatin organization and dynamics and cell type-specific expression in retinogenesis, terminal differentiation and postmitotic homeostasis. Modulation of DNA methylation and histone acetylation in vivo in mouse models can cause significant changes in retinal physiology. Research on RD and PR cell death in the past 10-15 years identified epigenetic modulation (e.g., using valproic acid) as a promising neuroprotective approach to delay PR cell death.

Histone deacetylase (HDAC) inhibitors are good candidates as therapeutics to ameliorate PR cell death in RP patients with certain mutations. Zhang, H., et al., Histone Deacetylases Inhibitors in the Treatment of Retinal Degenerative Diseases: Overview and Perspectives. J Ophthalmol, 2015. 2015: p. 250812. HDAC inhibitors are an emerging class of therapeutics with potential to cause chromatin conformation changes, which causes multiple cell type-specific effects in vitro and in vivo, such as growth arrest, modulation of gene expression, cell differentiation and postmitotic homeostasis. Ververis, K., et al., Histone deacetylase inhibitors (HDACIs): multitargeted anticancer agents. Biologics, 2013. 7: p. 47-60. There is evidence that valproic acid (VPA) induces histone H3 acetylation (Koriyama, Y., et al., Heat shock protein 70 induction by valproic acid delays photoreceptor cell death by N-methyl-N-nitrosourea in mice. J Neurochem, 2014. 130(5): p. 707-19), providing a link between VPA and HDAC inhibitor activities. Collectively, some selective compounds in this group of epigenetic drugs (impacting chromatin via histone modifications) are already approved by the Food and Drug Administration (FDA), thus providing a 10-15 year shortcut in approval by repurposing these compounds for use in ophthalmology (e.g., targeting retinal degeneration and blindness).

Likewise, DNA methylation processes are active in retinal cells undergoing terminal differentiation (i.e., cell fate choice commitment) (Rai, K., et al., Dnmt2 functions in the cytoplasm to promote liver, brain, and retina development in zebrafish. Genes Dev, 2007. 21(3): p. 261-6; Rai, K., et al., Zebra fish Dnmt1 and Suv39h1 regulate organ-specific terminal differentiation during development. Mol Cell Biol, 2006. 26(19): p. 7077-85), and create a retina-restricted pattern of gene expression (Mu, X., et al., A gene network downstream of transcription factor Math5 regulates retinal progenitor cell competence and ganglion cell fate. Dev Biol, 2005. 280(2): p. 467-81). DNA methylation is catalyzed by DNA methyltransferases DNMT1, DNMT3A and DNMT3B (Jaenisch, R. and A. Bird, Epigenetic regulation of gene expression: how the genome integrates intrinsic and environmental signals. Nat Genet, 2003. 33 Suppl: p. 245-54), and may differentially affect promoters of key transcription factors, such as NRL (Oh, E. C., et al., Transformation of cone precursors to functional rod photoreceptors by bZIP transcription factor NRL. Proc Natl Acad Sci USA, 2007. 104(5): p. 1679-84), Brn3b (Mu et al., Discrete gene sets depend on POU domain transcription factor Brn3b/Brn-3.2/POU4f2 for their expression in the mouse embryonic retina. Development, 2004. 131(6): p. 1197-210) or Math5, thereby influencing cell fate specification. Differential DNA methylation can affect, for example, the affinity of a transcription factor for its binding site, and/or recruitment/release of chromatin-binding repressors, such as REST/NRSF (Mu et al., supra), thereby providing a direct link between histone modification and DNA methylation machineries. In addition, the high level of DNMT1 in postmitotic retinal neurons (Nasonkin, I. O., et al., Distinct nuclear localization patterns of DNA methyltransferases in developing and mature mammalian retina. J Comp Neurol, 2011. 519(10): p. 1914-30; Nasonkin, I. O., et al., Conditional knockdown of DNA methyltransferase 1 reveals a key role of retinal pigment epithelium integrity in photoreceptor outer segment morphogenesis. Development, 2013. 140(6): p. 1330-41) and other CNS neurons, and association of DNMT1 with DNA double-stranded breaks and the DNA repair machinery (Ha, K., et al., Rapid and transient recruitment of DNMT1 to DNA double-strand breaks is mediated by its interaction with multiple components of the DNA damage response machinery. Hum Mol Genet, 2011. 20(1): p. 126-40) points to additional roles of DNMT1 in postmitotic neurons, which may be more relevant for therapeutic goals than the known classic role of DNMT1 as a methylator of the daughter DNA strand during DNA replication.

The PDE6B-mutant retinal organoids described in Examples 6 and 7 are used to evaluate a large number of epigenetic drugs (E-drugs), including those used for clinical trials (mentioned above), all epigenetic drugs in the Sigma-Aldrich catalog (about 30), and drugs that modulate DNA methylation and histone modification (e.g., methylation, acetylation). Epigenetic drugs are tested for their ability to promote PR survival, prevent PR cell death, and restore the integrity of the RPE-PR inner retinal neuron layers in PDE6B-mutant organoids, or in organoids that have been treated with MNU, 8-pCPT-PETcGMP or zaprinast; using the assays for neuroprotection described in Examples 6 and 7.

Each drug is tested in quadruplicate experiments (4 wells of a 96-well plate/each drug, 4-20 hESC-3D retinal tissue aggregates/each E-drug) and the best candidates are selected for further testing and for tests for synergy with other substances (e.g., trophic factors and/or mitogens). Criteria for selecting best candidates are preservation of PR cell numbers and synaptic connectivity; evaluated by voltage-sensitive dye activity, IHC, including mCherry, EGFP fluorescence and PR-specific Abs anti-RCVRN, anti-CRX, qRT-PCR with PR-specific genes, migration of trans synaptic tracer WGA-EGFP into PR synaptic partners, and PR flow cytometry sorting with an anti-CD73 antibody.

Best candidates as described above are tested for synergistic effects in promoting PR survival and synaptic connectivity to 2nd order neurons. In certain embodiments, two or more E-drugs are tested for synergy. In additional embodiments, E-drug(s) and trophic factors are tested for synergy. In additional embodiments, E-drug(s) and mitogens are tested for synergy.

In addition, whole transcriptome analysis of 3D in vitro retinal tissue, in the presence of one or more of the best E-drug candidates, is conducted to identify pathways (i.e., future drug targets), induced by the best neuroprotective E-drug candidate(s). Two sets of organoids with induced PR death (“Control”=no treatment, and “Experiment”=treated) are collected at 8, 16, 24 and optionally 36 weeks. Each sample is represented by organoids collected from 4 different wells of a 96-well plate.

Finally, whole-genome DNA methylation changes, and/or changes in histone methylation and/or acetylation are evaluated, using Chip-Seq-grade antibodies.

### Example 9: Evaluation of Drug-Mediated Shift in Photoreceptor Metabolism to Hypoxia-Like Conditions

Modulation of PR physiology with drugs affecting PR energy metabolism pathways (oxidative phosphorylation and glycolysis) is another very promising drug-mediated approach to augment PR survival. Interestingly, a number of epigenetic and energy metabolism modulation-based retinal therapy approaches converge on HIF1α-mediated hypoxia. Zhong, L., et al., The histone deacetylase Sirt6 regulates glucose homeostasis via Hif1alpha. Cell, 2010. 140(2): p. 280-93; Zhong, L. and R. Mostoslaysky, SIRT6: a master epigenetic gatekeeper of glucose metabolism. Transcription, 2010. 1(1): p. 17-21. Hypoxia shows a strong neuroprotective effect. Chen, B. and C. L. Cepko, HDAC4 regulates neuronal survival in normal and diseased retinas. Science, 2009. 323(5911): p. 256-9; Vlachantoni, D., et al., Evidence of severe mitochondrial oxidative stress and a protective effect of low oxygen in mouse models of inherited photoreceptor degeneration. Hum Mol Genet, 2011. 20(2): p. 322-35; Bull, N. D., et al., Use of an adult rat retinal explant model for screening of potential retinal ganglion cell neuroprotective therapies. Invest Ophthalmol Vis Sci, 2011. 52(6): p. 3309-20. There is a critical need to rapidly evaluate a large number of promising small molecules impacting these metabolic pathways to design new drug regimens for attenuating PR cell death.

Recent research on RD and PR cell death has identified metabolic changes resembling the hypoxic state, in the retinal metabolome, as promising neuroprotective approaches to delay PR cell death. Vlachantoni, D., et al., Evidence of severe mitochondrial oxidative stress and a protective effect of low oxygen in mouse models of inherited photoreceptor degeneration. Hum Mol Genet, 2011. 20(2): p. 322-35; Thiersch, M., et al., The hypoxic transcriptome of the retina: identification of factors with potential neuroprotective activity. Adv Exp Med Biol, 2008. 613: p. 75-85; Thiersch, M., et al., Analysis of the retinal gene expression profile after hypoxic preconditioning identifies candidate genes for neuroprotection. BMC Genomics, 2008. 9: p. 73.

Aerobic glycolysis (the Warburg effect), a distinct feature of cancer and embryonic cell metabolism, is also typical in mammalian retina. The mammalian neural retina has high energy demands to keep the neurons in an excitable state for phototransduction, neurotransmission, and maintenance of normal homeostatic functions. The outer retina has the highest level of glycolytic activity. Most aerobic glycolysis takes place in the outer retina, mainly in the photoreceptors. Graymore (1960) observed a greater than 50% reduction in glycolytic activity within dystrophic rat retinas lacking photoreceptor cells, when compared to normal rat retina. Wang et al. (1997) reported glucose consumptions in pig retina in vivo by measuring the arteriovenous differences in glucose concentrations. The inner retina metabolized 21% of the glucose via glycolysis and 69% via oxidative metabolism, in contrast to the outer retina that metabolized 61% of the glucose via aerobic glycolysis and only 12% via oxidative metabolism.

The different retinal layers exhibit differential oxygen consumption in mammalian retina. The deep inner plexiform layer, the outer plexiform layer and the inner segments of photoreceptor cells have much higher oxygen consumption, compared to the outer segments of the photoreceptors and the outer nuclear layers in vascularized mammalian retina. Though the loss of oxygenation of retinal tissue (anoxia, such as in stroke or retinal detachment) leads to PR cell death, pharmacological modulation of PR metabolism to mimic the hypoxic state is neuroprotective and therapeutic. See, e.g., Vlachantoni, D. et al., Evidence of severe mitochondrial oxidative stress and a protective effect of low oxygen in mouse models of inherited photoreceptor degeneration. Hum Mol Genet, 2011. 20(2): p. 322-35; and Bull, N. D. et al., Use of an adult rat retinal explant model for screening of potential retinal ganglion cell neuroprotective therapies. Invest Ophthalmol Vis Sci, 2011. 52(6): p. 3309-20. The isolated rat retina can robustly support electrical activity in PRs anaerobically if glucose is abundant. In these conditions the electrical activity can be maintained at 80% for 30 min of anoxia; then falls to 40% of the aerobic value when the glucose supply is reduced. To summarize, while both oxidative phosphorylation and aerobic glycolysis are needed for optimal retinal metabolism and functioning (and RP disease may be induced in cases in which oxidative phosphorylation is completely abrogated), shifting the homeostatic balance of oxidative phosphorylation versus glycolysis to mimic conditions of very low oxygen concentration, just short of anoxia, does seem to be therapeutic and is a promising approach to protect and maintain PRs.

Because metabolic changes, including hypoxia, can ameliorate PR cell death, modulators of PR metabolism are useful in the treatment of retinal degeneration. Accordingly, the experimental system described in Examples 6 and 7 (i.e., human retinal organoids containing a mutation in the PDE6B gene) is used to screen test substances and/or test genes for their effect on PR metabolism. As noted previously, a number of epigenetic and energy metabolism modulation pathway converge on HIF1α-mediated hypoxia, which shows a strong neuroprotective effect and regulates mitochondrial genes encoding electron transport chain proteins. HIF1alpha and HDAC regulation seem also to be tightly connected, providing a link between epigenetic modulators and modulators of metabolism. Thus, epigenetic modulators and modulators of metabolism, identified by the screens described herein, are also screened in combination for synergistic activity in prevention PR cell death.

To this end, several small molecules known to shift the metabolic state of cells from the oxidative phosphorylation (OXPHOS) and glycolysis mode toward hypoxia-like conditions (Metabolic, or M-drugs, e.g. 1,4-dihydrophenonthrolin-4-one-3-carboxylic acid (1,4-DPCA), a PHD (prolyl hydrohylase) inhibitor that stabilizes HIF-1α) are evaluated for their ability to promote PR survival and synaptic activity in PDE6B-mutant 3D retinal organoids. Whole transcriptome analysis is conducted to delineate neuroprotective changes in the PR transcriptome induced by such M-drugs and identify pathways (i.e., future drug targets), induced by neuroprotective M-drug compounds.

The best M-drug candidates are tested for synergistic effects in promoting PR survival and synaptic connectivity to 2nd order neurons. In certain embodiments, two or more M-drugs are tested for synergy. In additional embodiments, M-drug(s) and E-drug(s) are tested for synergy. In additional embodiments, M-drug(s) and trophic factors are tested for synergy. In additional embodiments, M-drug(s) and mitogens are tested for synergy.

### Example 10: Comparison of Developmental Dynamics in Human Fetal Retina and hESC-3D Retinal Tissue

Although transplantation of human fetal retinal tissue has been shown to restore vision in some animals with retinal degeneration and in some patients with RP, fetal retina is limited in its availability and there are ethical constraints associated with its use. The hESC-3D retinal tissue (retinal organoids) derived from human pluripotent stem cells (hPSCs) share many similarities with human fetal retina and provide a surprising replacement for fetal retinal tissue to treat retinal diseases, injuries and disorders.

This Example demonstrates the similarities in distribution and gene expression of molecular markers in developing human fetal retina and hESC-3D retinal tissue. Immunophenotyping analysis, immunohistochemistry and RNA-seq methods were used to assess the similarities between fetal retina and hESC-3D retinal tissue. Results showed a high correlation in gene expression profiles between human fetal retina and hESC-3D retinal tissue, providing evidence of the use of these materials usefulness to treat retinal diseases, injuries and disorders. Immunohistochemical profiling of developing human fetal retinal tissue at 8-16 weeks showed strong expression of retinal pigment epithelium (RPE) markers (EZRIN, Beta-catenin), retinal progenitor markers (OTX2, CRX, PAX6), photoreceptor marker (RCVRN), amacrine marker (CALB2) and ganglion marker (BRN3B).

Immunophenotyping by Flow Cytometric Analysis

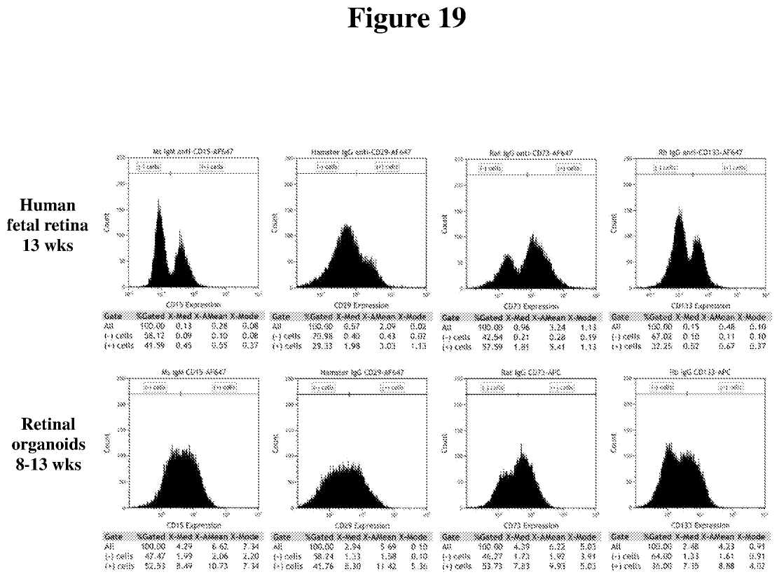

FIG. 19 shows immunophenotyping results of 13-week old human fetal retina and 8-week old hESC-3D retinal tissue. Cells were first dispersed into a uniform single-cell suspension using a papain digestion protocol, as previously described (Maric D, Barker J L. Fluorescence-based sorting of neural stem cells and progenitors. Curr Protoc Neurosci. 2005;Chapter 3 p. Unit 3 18). The resulting mixture of cells was immunolabeled with the following cocktail of lineage-selective surface markers: rabbit IgG anti-CD133, mouse IgM anti-CD15 (Santa Cruz Biotechnology, Santa Cruz, Calif.), mouse IgG1 anti-CD29 (BD Biosciences, San Jose, Calif.), and a mixture of tetanus toxin fragment C (TnTx)-anti-TnTx mouse IgG2b, which was prepared in-house as previously described (Maric and Barker, 2005). Primary immunoreactions were visualized using the following fluorophore-conjugated goat secondary antibodies: anti-rabbit IgG-FITC, anti-mouse IgM-PE (Jackson ImmunoResearch Laboratories Inc., West Grove, Pa.), anti-mouse IgG1-PE/Texas Red (PE/TR), and anti-mouse IgG2b-PE/Cy5 (Invitrogen, Carlsbad, Calif.). After surface labeling, cells were stained with 1 mg/ml DAPI to discriminate between live (DAPI-negative) and dead (DAPI-positive) cells. Quantitative immunophenotyping of cell populations was carried out using the FACSVantage SE flow cytometer (BD Biosciences), as previously described (Maric and Barker, 2005). Briefly, the fluorescence signals emitted by FITC, PE, PE/TR and PE/Cy5 on individual cells were excited using an argon-ion laser tuned to 488 nm and the resulting fluorescence emissions collected using bandpass filters set at 530±30 nm, 575±25 nm, 613±20 nm and 675±20 nm, respectively. DAPI-labeled cells were excited using a broad UV (351-364 nm) laser light and the resulting emission signals captured with a bandpass filter set at 440±20 nm. Cell Quest Acquisition and Analysis software (BD Biosciences) was used to acquire and quantify the fluorescence signal distributions and intensities from individual cells, to electronically compensate spectral overlap of individual fluorophores and to set compound logical electronic gates used for cell analysis.

CD15 has been described as a marker of retinal interneurons including amacrine and bipolar cells (Jakobs, T. C., Ben, Y., and Masland, R. H. (2003). CD15 immunoreactive amacrine cells in the mouse retina. J. Comp. Neurol. 465, 361-371). As shown in FIG. 19, there is a similarity in the number of cells with second order neurons (e.g., interneurons, including amacrine and bipolar neurons) in hESC-3D retinal tissue (52.53%) and human fetal retina (41.59%). CD73 is a surface marker present on developing and mature photoreceptors. The results illustrated in FIG. 19 show that 53.73% of cells in the hESC-3D retinal tissue and 57.59% of the cells in 13-week old human fetal retinal tissue are photoreceptors. FIG. 19 also shows a similarity in the presence of CD133 (a marker of symmetric division and major neural stem and progenitor cell marker) in hESC-3D retinal tissue (36.00%) and human fetal retina (32.25%). This data demonstrates the similarity in the number of young retinal cells that are dividing symmetrically and shows that the differentiation state of the developing hESC-3D retinal tissue and human fetal retina are very close at these time points.

Transcriptome Analysis

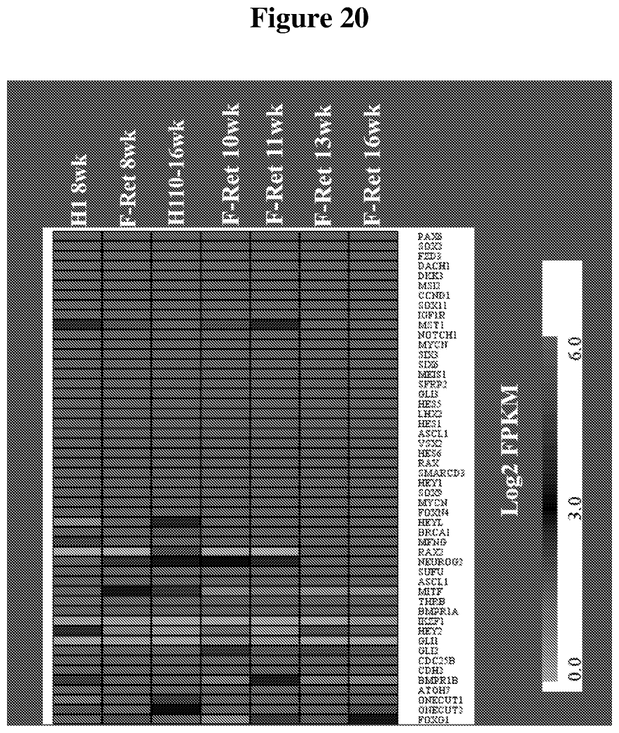

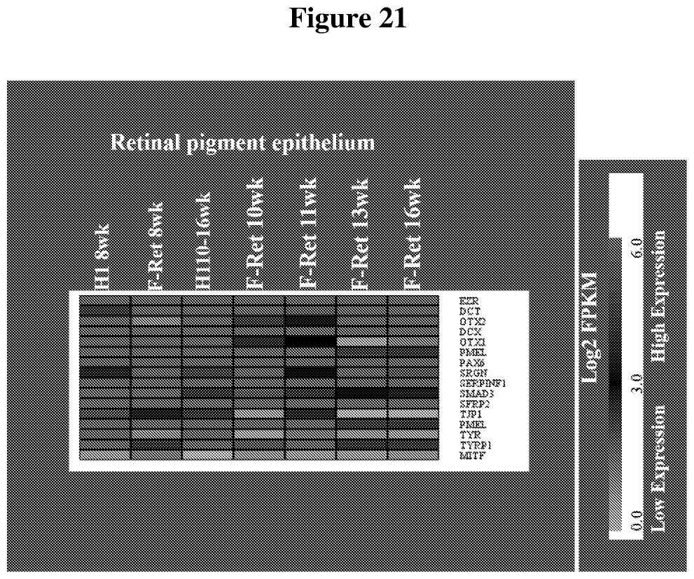

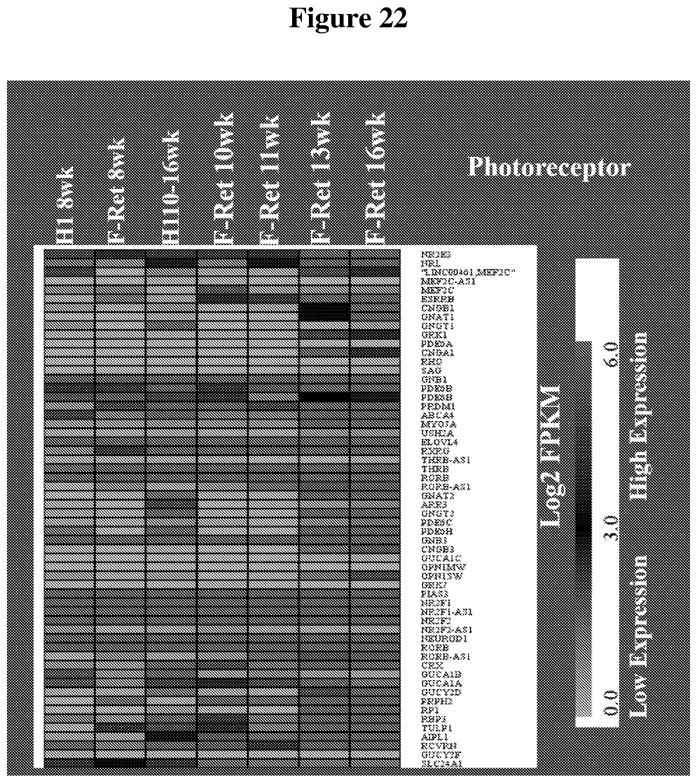

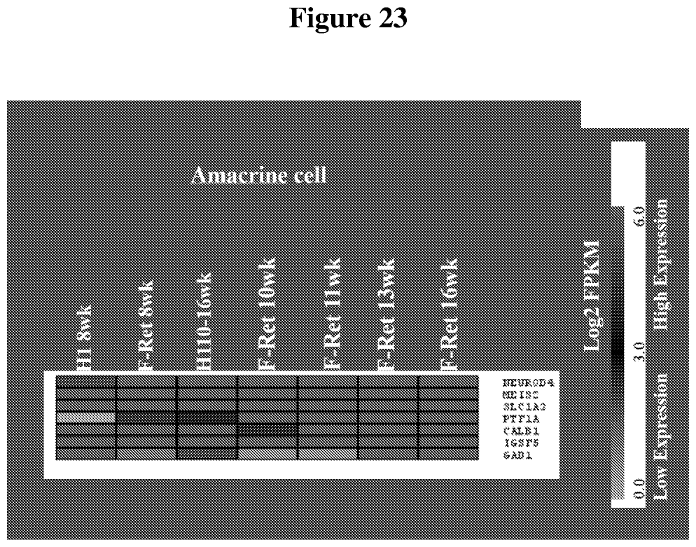

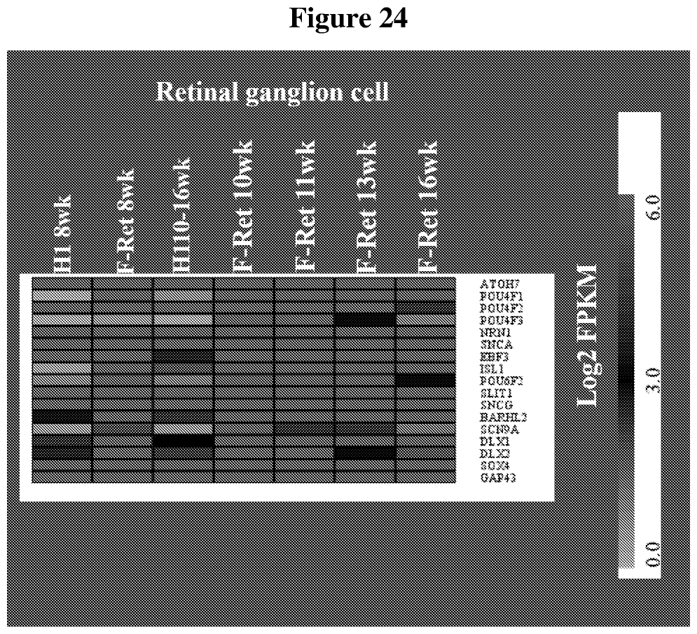

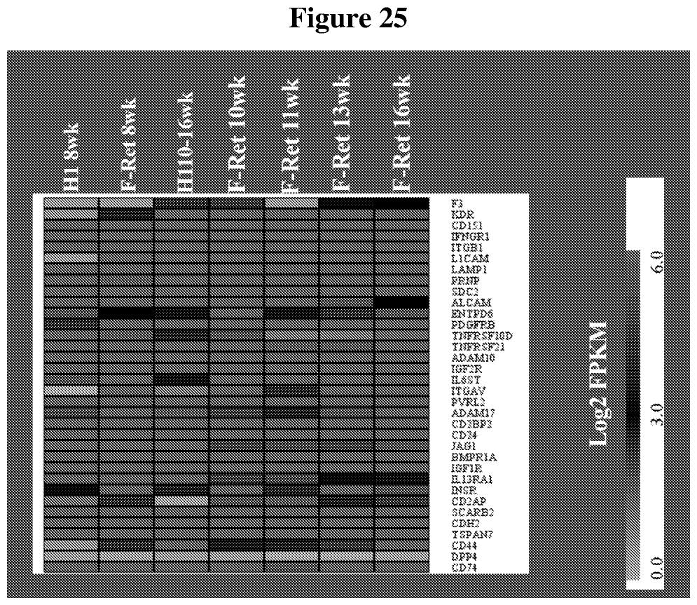

Transcriptome analysis utilizing RNA sequencing was performed by BGI according to our specifications. The data from the transcriptome profiling of hESC-3D retinal tissue and human fetal retina is presented in FIG. 20 through FIG. 25. FIG. 20 is a heat map showing a comparison of retinal progenitor cell expression profiles for hESC-3D retinal tissue (H1) and human fetal retina (F-Ret) at different time points. The data show a high similarity in progenitor specific gene expression among hESC-3D retinal tissue at 8 weeks and human fetal retina at 8 and 10 weeks. FIG. 21 shows a heat map comparing RPE specific gene expression in hESC-3D retinal tissue versus human fetal retina at different time points. The low level of expression in the human fetal retina samples was expected because human fetal retina samples are composed of “neural retina” that has been separated from the layer of RPE. In contrast, the hESC-3D retinal tissue shows higher expression of RPE-specific genes such as TYR and TYRP, indicating the presence of an RPE layer in hESC-3D retinal tissue. FIG. 22 shows a heat map depicting the pattern of photoreceptor-specific gene expression, which is very similar in hESC-3D retinal tissue and human fetal retinal tissue. FIG. 23 and FIG. 24 show heat maps that illustrate the similarities in gene expression profiles for amacrine cells and retinal ganglion cells (RGC) (respectively) among hESC-3D retinal tissue and human fetal retinal tissue at different time points. Finally, FIG. 25 shows a heat map displaying similar cell surface marker gene expression profiles for hESC-3D retinal tissue and human fetal retinal tissue.

Immunohistochemical Characterization of Retinal Sections: 10-Week Old Human Fetal Retina and 8-Week Old hESC-3D Retinal Tissue

Human fetal retina and hESC-derived retinal tissue aggregates growing in adherent condition were fixed in fresh ice-cold paraformaldehyde (4% PFA; Sigma-Aldrich) for 15 minutes (min), rinsed with 1× phosphate-buffered saline (PBS), and washed thrice in ice-cold PBS (5 min each). The aggregates were cryoprotected in 20% sucrose (prepared in PBS, pH 7.8), and then 30% sucrose (until tissue sank), and snap-frozen (dry ice/ethanol bath) in optimum cutting temperature (OCT) embedding material (Tissue-Tek). hESC-derived retinal tissue aggregates were serially sectioned at 12 μm. The sections were first permeabilized with 0.1% Triton X-100/PBS (PBS-T) at room temperature for 30 min, followed by 1 h of incubation in blocking solution [5% preimmune normal goat serum (Jackson Immunoresearch) and 0.1% PBS-T] at room temperature, and then were incubated with primary antibodies diluted in blocking solution at 4° C. overnight. The following day sections were washed thrice (10-15 min each time) with PBS-T, and then incubated with the corresponding secondary antibodies (Alexa Fluor 568 goat anti-mouse, Alexa Fluor 488 goat anti-rabbit, 1:1,000, or vice versa) at room temperature for 45 min. The slides were washed thrice with 0.1% PBS-T solution, incubated with 4′, 6-diamidino-2-phenylindole (DAPI) solution (1 μg/mL) for 10 min, and then washed again with 0.1% PBS-T solution. As a negative control for primary antibody-specific binding, we stained tissue sections with secondary antibodies only. The specimens were mounted with ProLong Gold Antifade medium (Life Technologies) and examined using a Nikon Eclipse Ni epifluorescent microscope with ZYLA 5.5 sCMOS (ANDOR Technologies) black and white charge-coupled device high-speed camera or Olympus FluoView FV1000 confocal microscope (Olympus). Antibodies are listed in Table S2.

SUPPLEMENTARY TABLE S2
LIST OF PRIMARY ANTIBODIES

| Target cells | Target proteins/epitope | Host | Dilutions | Vendor |
|---|---|---|---|---|
| HESC marker | Oct3/4 | Rabbit | 1:500 | Abcam |
| | Nanog | Rabbit | 1:1,000 | Abcam |
| RPE marker | Ezrin | Mouse | 1:250 | Abcam |
| | NHERF1-H100 | Rabbit | 1:250 | Santacruz |
| Eye field marker | RAX | Rabbit | 1:250 | Abcam |
| | OTX2 | Rabbit | 1:250 | Abcam |
| | MAP2 | Mouse | 1:500 | Abcam |
| | PAX6 | Rabbit | 1:500 | Covance |
| | CRX | Mouse | 1:500 | Abnova |
| | LHX2 | Rabbit | 1:250 | Gift from Edwin Monuki |
| | CHX10 | Rabbit | 1:500 | Gift from Connie Cepko |
| Cell proliferation | Ki67 | Rabbit | 1:500 | Abcam |
| | Ki67 | Mouse | 1:500 | BD Pharm |
| Photoreceptor | Recoverin | Rabbit | 1:500 | Millipore |
| | HNu | Mouse | | Chemicon |
| Horizontal Axons | NF200 | Rabbit | 1:500 | Chemicon |
| Amacrine | Calretinin | Rabbit | 1:250 | Millipore |
| | LGR5 | Rabbit | 1:250 | Abgent |
| Ganglion | Brn3b | Rabbit | 1:250 | gift front Tudor |
| | Brn3a | Rabbit | 1:250 | Millipore |
| | Synaptophysin | Mouse | 1:250 | Chemicon |
| Stem cell | TERT | Rabbit | 1:250 | Abgent |
| | DCAMLK1 | Rabbit | 21:250 | Abcam |

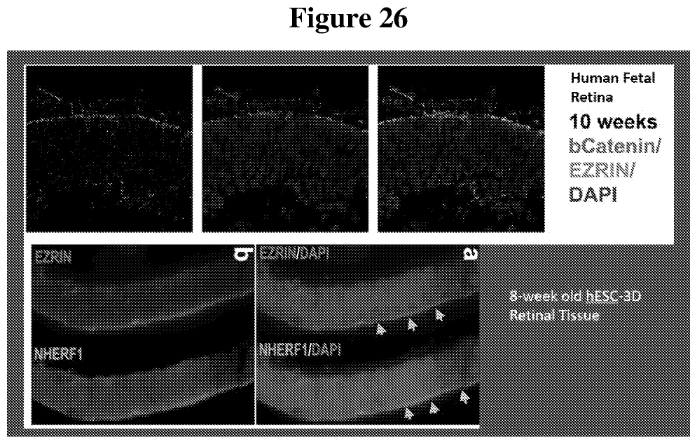

FIG. 26 through FIG. 32 show images of immunohistochemical characterization performed on both human fetal retina and hESC-3D retinal tissue. The images in FIG. 26 through FIG. 32 illustrate the similar cell marker distribution of many retinal and RPE markers for human fetal retina and hESC-3D retinal tissue. In FIG. 26, the presence of the RPE marker, EZRIN, can be seen in the apical surface of 10-week old human fetal retina and 8-week old hESC-3D retinal tissue. These images show the RPE as a single layer with a similar cell marker distribution in both the 10-week old human fetal retina and 8-week old hESC-3D retinal tissue.

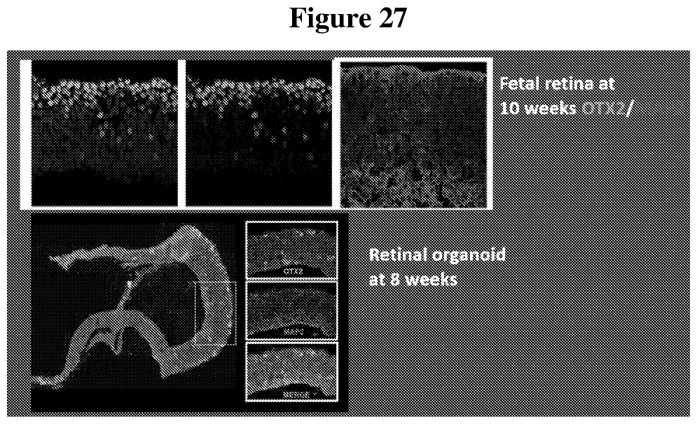

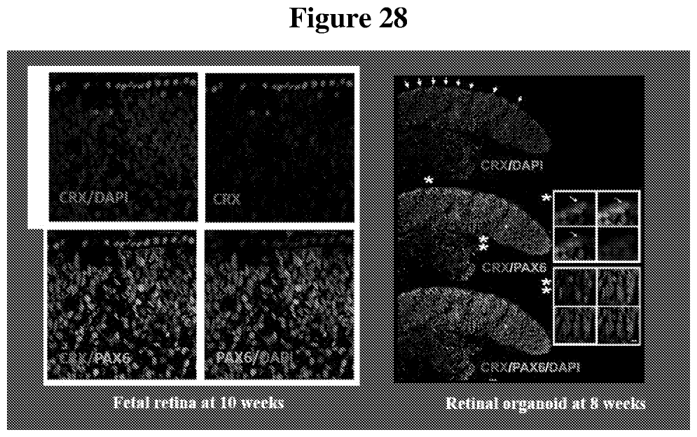

Referring to FIG. 27, OTX2 is a nuclear marker for photoreceptors at the 8-week to 10-week stage of retinal development. MAP2 is a marker for RCGs and amacrine neurons at the 8-week to 10-week stage of retinal development. The images presented in FIG. 27 demonstrate that the distribution of these markers is very similar in the 10-week old human fetal retina and 8-week old hESC-3D retinal tissue.

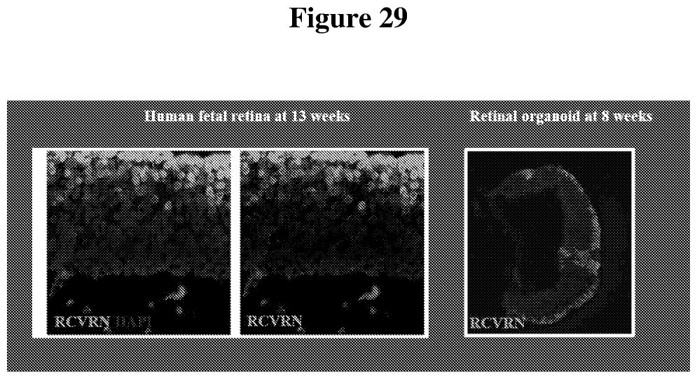

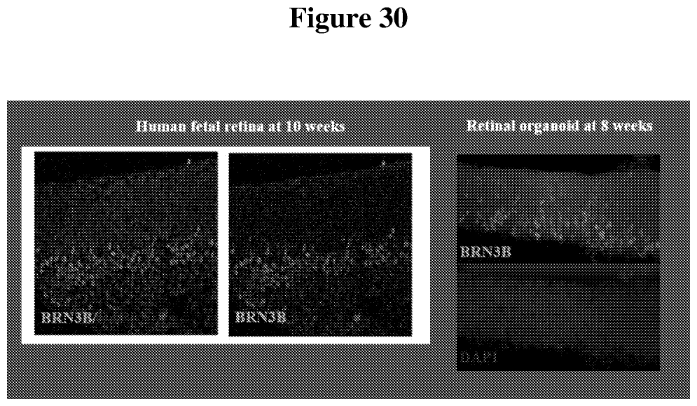

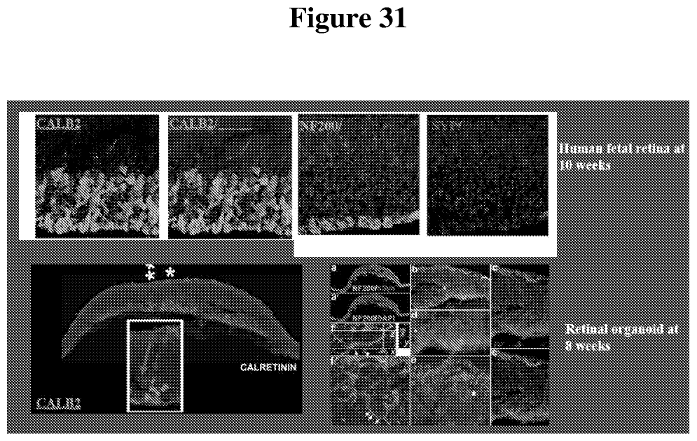

FIG. 28 shows images of the pattern of cell marker distribution of the CRX (cone rod homeobox) marker, which is a major early photoreceptor marker, and the PAX6 marker for retinal progenitor cells and RGCs. The distribution patters in the 10-week old human fetal retina and 8-week old hESC-3D retinal tissue are comparable for these two markers. Highly similar patterns of marker distribution can also be seen in FIG. 29 for the Recoverin marker, which is present in young photoreceptors in the 13-week old human fetal retinal tissue and in 8-week old hESC-3D retinal tissue. Similar patterns can also be seen in 10 to 13-week old hESC-3D retinal tissue (data not shown). Comparison of the immunostaining of the BRN3B marker for RGCs in 10-week old human fetal retinal tissue and in 8-week old hESC-3D retinal tissue also shows a similarity in cell marker distribution patterns at the basal side, opposite the RPE layer as seen in FIG. 30. A highly similar distribution pattern for cells labeled with CALB2 (calretinin) in 10-week old human fetal retinal tissue and in 8-week old hESC-3D retinal tissue can be seen in FIG. 31.

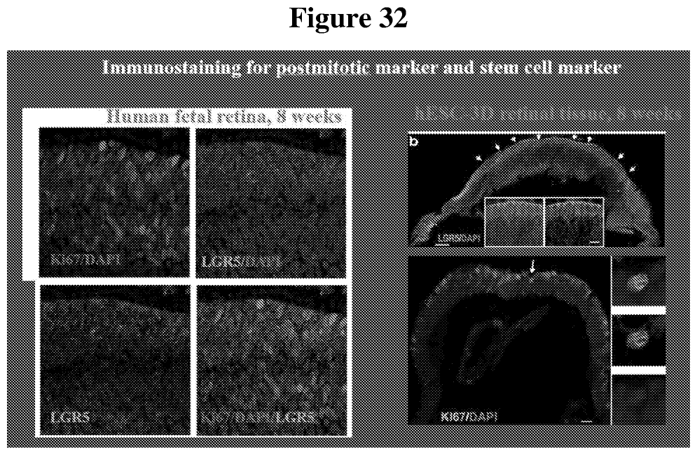

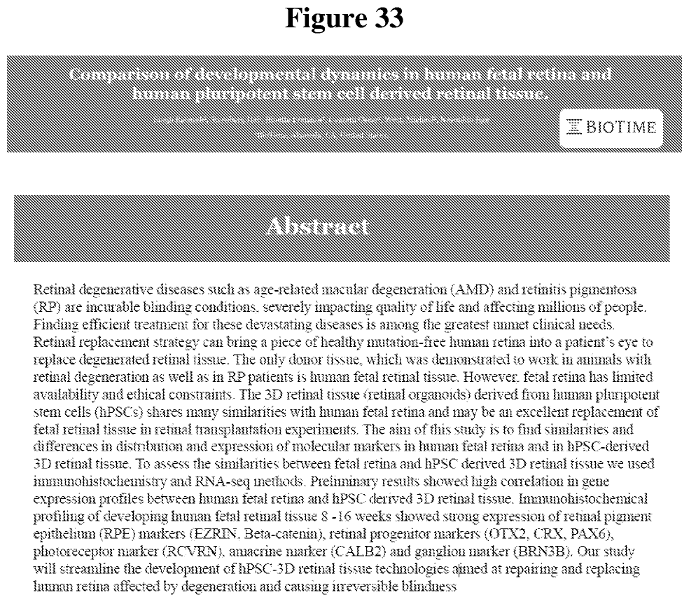

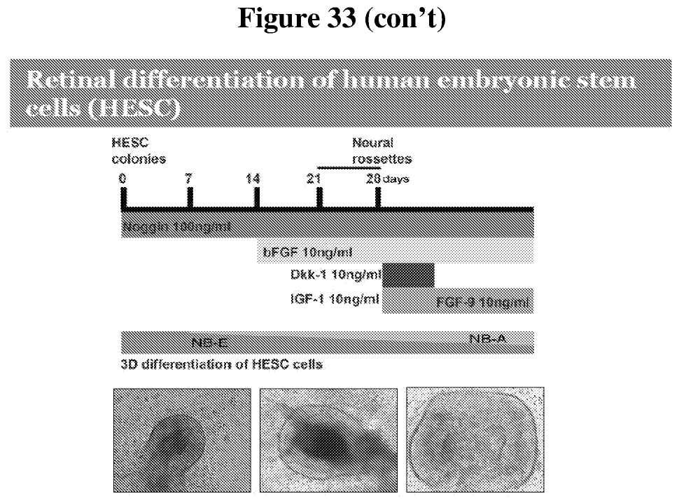

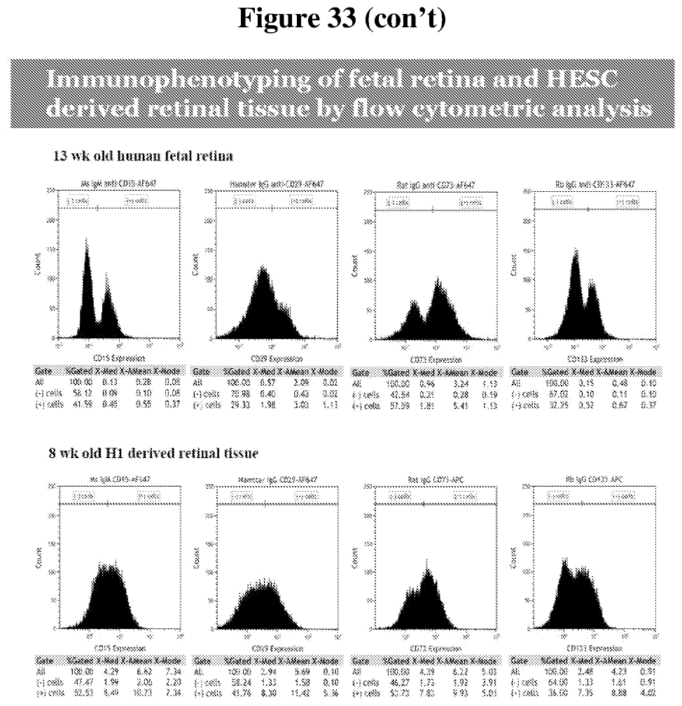

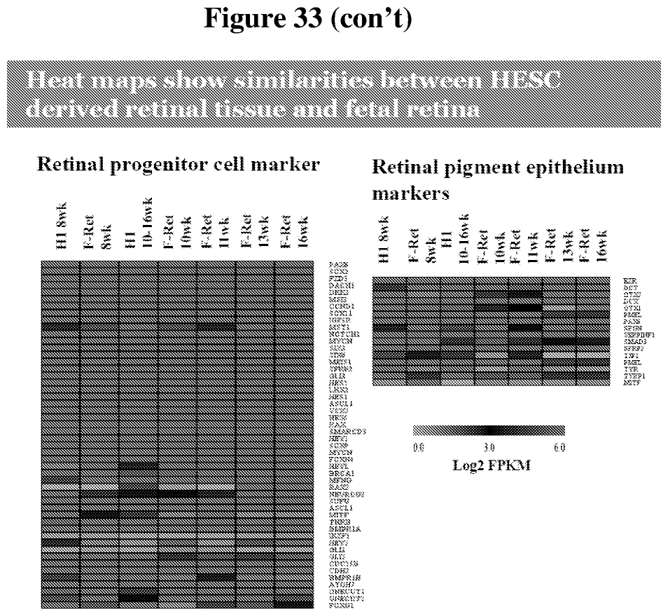

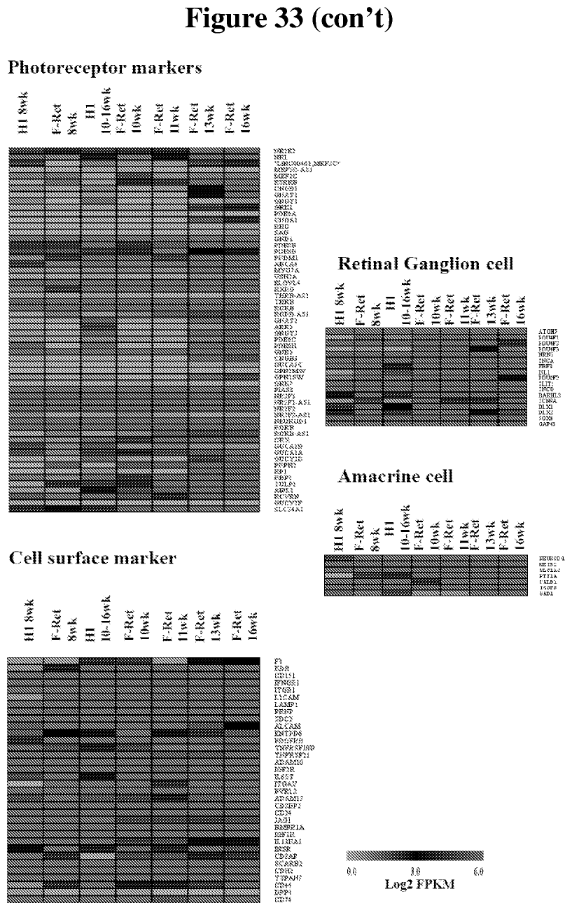

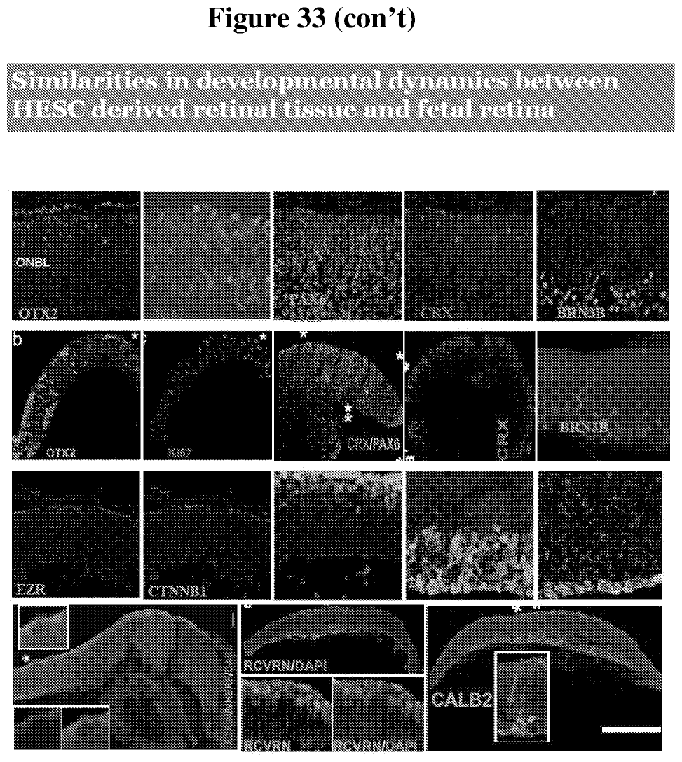

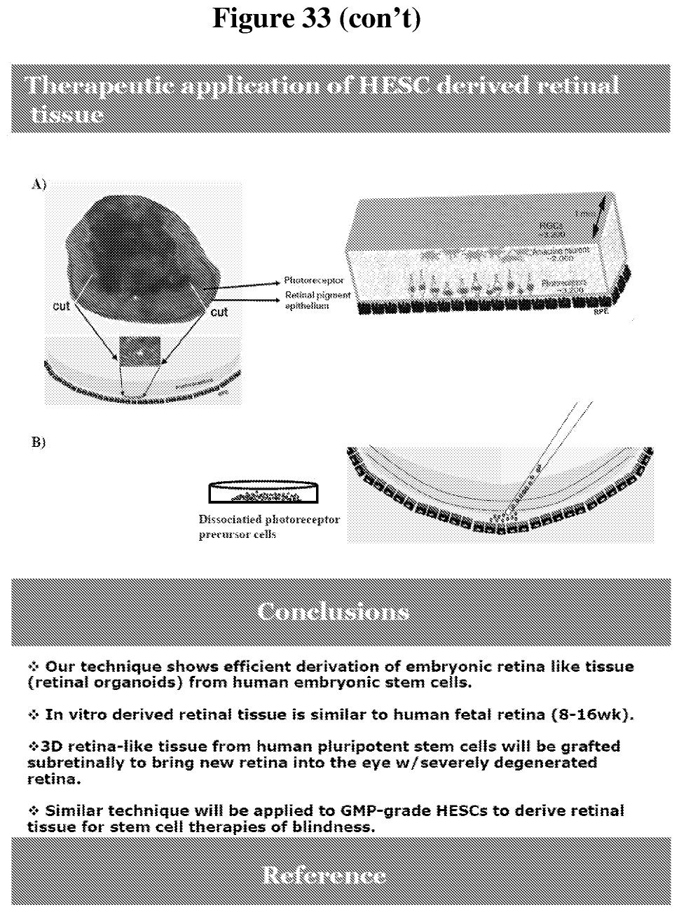

FIG. 32 shows the distribution of cells labeled with the LGR5 marker, which shows dividing stem cells (Wnt-signaling, postmitotic marker). The LGR5 immunostaining images show that stem cells are only dividing where expected in both the 10-week old human fetal retinal tissue and in 8-week old hESC-3D retinal tissue. FIG. 33 provides a summary of the comparison of developmental dynamic in human fetal retina and human pluripotent stem cell derived retinal tissue discussed herein.

These results demonstrate that hESC-3D retinal tissue at age 6 to 8-weeks is very similar to 8 to 10-week old human fetal retina (based on the distribution of CRX, OTX2, BRN3B, MAP2, SOX2, PAX6, LGR5, EZRIN and other markers) and the usefulness of the tissue to treat retinal diseases, injuries and disorders.

### Example 11: Transplantation of hESC-3D Retinal Tissue into Subretinal Space of Blind Rd Rats

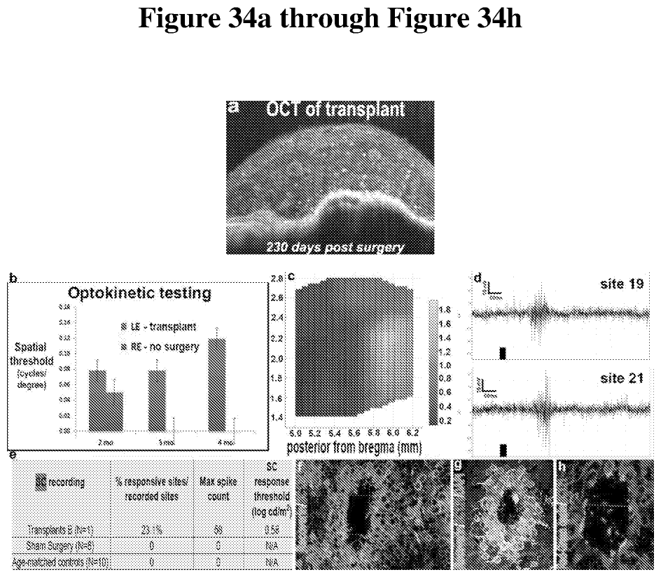

hESC-3D retinal tissue was dissected into sheets, and transplanted into blind SD-Foxnl Tg(S334ter)3Lav (RD nude), age P25-30 rats. Transplantation was performed as described by Seiler et al. for human fetal retina (Aramant, R. B. and M. J. Seiler, Transplanted sheets of human retina and retinal pigment epithelium develop normally in nude rats. Exp Eye Res, 2002. 75(2): p. 115-25), using the specialty surgical tool described in U.S. Pat. No. 6,159,218. Three grafts were detected by Optical Coherence Tomography (OCT) after 230 days (FIG. 34a). The rats were tested for visual acuity improvements using optokinetic (OKN) (optokinetic drum (Douglas, R. M., et al., Independent visual threshold measurements in the two eyes of freely moving rats and mice using a virtual-reality optokinetic system. Vis Neurosci, 2005. 22(5): p. 677-84) at 2, 3, and 4 months after surgery (FIG. 34b)). The results showed significant improvement in transplanted animal vs. control (“sham surgery”, also “no surgery”) groups. Visual responses in superior colliculus (electrophysiological recording) were evaluated at 8.3 months post-surgery in one animal and demonstrated responses to light. No responses to light were detected in RD age-matched control group and sham surgery RD group (FIG. 34c shows a spike count heat map and FIG. 34d shows examples of traces). The grafts also demonstrated the presence of mature PRs and other retinal cell types (FIG. 34e through FIG. 340 and were immunoreactive to human (but not rat)-specific antibody SC121.

From the description herein, it will be appreciated that that the present disclosure encompasses multiple embodiments which include, but are not limited to, the following:

In vitro retinal tissue, wherein the retinal tissue: (a) comprises a disc-like three-dimensional shape; and (b) comprises a concentric laminar structure comprising one or more of the following cellular layers extending radially from the center of the structure: (i) a core of retinal pigmented epithelial (RPE) cells, (ii) a layer of retinal ganglion cells (RGCs), (iii) a layer of second-order retinal neurons (inner nuclear layer), (iv) a layer of photoreceptor (PR) cells, and (v) a layer of retinal pigmented epithelial cells.

The in vitro retinal tissue of any previous embodiment, wherein any one or more of the layers comprises a single cell thickness.

The in vitro retinal tissue of any previous embodiment, wherein any one or more of the layers comprises a thickness greater than a single cell.

The in vitro retinal tissue of any previous embodiment, wherein any one or more of the layers further comprises progenitors to the cells in the layer.

The in vitro retinal tissue of any previous embodiment, wherein one or more of the cells express LGR5.

The in vitro retinal tissue of any previous embodiment, wherein one or more of the cells express one or more genes selected from the group consisting of RAX, OTX2, LHX2, CHX10, MITF, PAX6, CRX, Recoverin (RCVRN) and BRN3A.

The in vitro retinal tissue of any previous embodiment, wherein one or more of the cells express one or more of the SOX1, SOX2, OTX2 and FOXG1 genes.

The in vitro retinal tissue of any previous embodiment, wherein one or more of the cells express one or more of the RAX, LHX2, SIX3, SIX6 and PAX6 genes.

The in vitro retinal tissue of any previous embodiment, wherein one or more of the cells express one or more of the NEURO-D1, ASCL1 (MASH1), CHX10 and IKZF1 genes.

The in vitro retinal tissue of any previous embodiment, wherein one or more of the cells express one or more genes selected from the group consisting of CRX, RCVRN, NRL, NR2E3, PDE6B, and OPN1SW.

The in vitro retinal tissue of any previous embodiment, wherein one or more of the cells express one or more genes selected from the group consisting of MATH5, ISL1, BRN3A,

BRN3B, BRN3C and DLX2.

The in vitro retinal tissue of any previous embodiment, wherein one or more of the cells express one or more genes selected from the group consisting of PROX1, PRKCA, CALB1 and CALB2.

The in vitro retinal tissue of any previous embodiment, wherein one or more of the cells express one or more genes selected from the group consisting of MITF, TYR, TYRP, RPE65, DCT, PMEL, Ezrin and NHERF1.

The in vitro retinal tissue of any previous embodiment, wherein one or more of the cells do not express the NANOG and OCT3/4 genes.

The in vitro retinal tissue of any previous embodiment, wherein the cells do not express markers of endoderm, mesoderm, neural crest, astrocytes or oligodendrocytes.

A composition comprising the in vitro retinal tissue of claim 1.

The composition of any previous embodiment, further comprising a hydrogel.

The composition of any previous embodiment, wherein the composition is a cell culture.

The cell culture of any previous embodiment, wherein culture is conducted under adherent conditions.

The cell culture of any previous embodiment, further comprising a hydrogel.

A method for making retinal tissue in vitro, the method comprising: (a) culturing pluripotent cells, under adherent conditions, in the presence of noggin for a first period of time; (b) culturing the adherent cells of (a) in the presence of noggin and basic fibroblast growth factor (bFGF) for a second period of time; (c) culturing the adherent cells of (b) in the presence of Noggin, bFGF, Dickkopf-1 (Dkk-1) and insulin-like growth factor-1 (IGF-1) for a third period of time; and (d) culturing the adherent cells of (c) in the presence of Noggin, bFGF, and fibroblast growth factor-9 (FGF-9) for a fourth period of time.

The method of any previous embodiment, wherein the concentration of noggin is between 50 and 500 ng/ml; the concentration of bFGF is between 5 and 50 ng/ml; the concentration of Dkk-1 is between 5 and 50 ng/ml; the concentration of IGF-1 is between 5 and 50 ng/ml and the concentration of FGF-9 is between 5 and 50 ng/ml.

The method of any previous embodiment, wherein the concentration of noggin is 100 ng/ml; the concentration of bFGF is 10 ng/ml; the concentration of Dkk-1 is 10 ng/ml; the concentration of IGF-1 is 10 ng/ml and the concentration of FGF-9 is 10 ng/ml.

The method of any previous embodiment, wherein the first period of time is between 3 and 30 days; the second period of time is between 12 hours and 15 days; the third period of time is between 1 and 30 days; and the fourth period of time is 7 days to one year.

The method of any previous embodiment, wherein the first period of time is 14 days; the second period of time is 14 days; the third period of time is 7 days; and the fourth period of time is 7 days to 12 weeks.

The method of any previous embodiment, wherein, in step (a), the pluripotent cells are initially cultured in a first medium that supports stem cell growth and, beginning at two to sixty days after initiation of culture, a second medium that supports growth of differentiated neural cells is substituted for the first medium at gradually increasing concentrations until the culture medium contains 60% of the second medium and 40% of the first medium.

The method of any previous embodiment, wherein, the first medium is Neurobasal® medium and the second medium is Neurobasal®-A medium; further wherein the second medium is substituted for the first medium beginning seven days after initiation of culture; and further wherein the culture medium contains 60% of the second medium and 40% of the first medium at 6 weeks after initiation of culture.

The method of any previous embodiment, wherein the fourth period of time is between 3 months and one year.

The method of any previous embodiment, wherein the pluripotent cell is a human embryonic stem cell (hESC) or an induced pluripotent stem cell (iPSC).

A method for treating retinal degeneration in a subject, the method comprising administering, to the subject, the in vitro retinal tissue of any previous embodiment, or a portion thereof.

The method of any previous embodiment, wherein administration is to the eye of the subject.

The method of any previous embodiment, wherein the administration is intravitreal.

The method of any previous embodiment, wherein the administration is subretinal.

The method of any previous embodiment, wherein the retinal degeneration occurs in retinitis pigmentosa (RP).

The method of any previous embodiment, wherein the retinal degeneration occurs in age-related macular degeneration (AMD).

The method of any previous embodiment, wherein the in vitro retinal tissue, or portion thereof, is administered together with a hydrogel.

The in vitro retinal tissue of any previous embodiment, wherein the cells comprise a first exogenous nucleic acid, wherein the first exogenous nucleic acid comprises: (a) a recoverin (RCVN) promoter; (b) sequences encoding a first fluorophore; (c) an internal ribosome entry site (IRES); and (d) sequences encoding a fusion polypeptide comprising an anterograde marker and a second fluorophore.

The in vitro retinal tissue of any previous embodiment, wherein the first fluorophore is mCherry.

The in vitro retinal tissue of any previous embodiment, wherein the anterograde marker is wheat germ agglutinin (WGA).

The in vitro retinal tissue of any previous embodiment, wherein the second fluorophore is enhanced green fluorescent protein (EGFP).

The in vitro retinal tissue of any previous embodiment, wherein the cells further comprise a second exogenous nucleic acid, wherein the second exogenous nucleic acid comprises: (a) a tetracycline-inducible recoverin (RCVN) promoter (tet-on pRCVRN); (b) Frt sequences; (c) an internal ribosome entry site (IRES); and (d) sequences encoding a marker gene.

The in vitro retinal tissue of any previous embodiment, wherein the marker gene is enhanced cyan fluorescent protein (ECFP).

The in vitro retinal tissue of any previous embodiment, wherein the second exogenous nucleic acid further comprises sequences encoding a test gene located between the Frt sequences.

A method for screening for a test substance that enhances synaptic connectivity between retinal cells, the method comprising: (a) incubating the in vitro retinal tissue of claim 37, in the presence of the test substance; and (b) testing for synaptic activity; wherein an increase in synaptic activity in cultures in which the test substance is present, compared to cultures in which the test substance is not present, indicates that the test substance enhances synaptic connectivity.

The method of any previous embodiment, wherein the retinal cells are PRs and second-order retinal neurons.

The method of any previous embodiment, wherein the test substance is selected from the group consisting of an exosome preparation, conditioned medium, a protein, a polypeptide, a peptide, a low molecular weight organic molecule, and an inorganic molecule.

The method of any previous embodiment, wherein the exosomes are obtained from a pluripotent cell.

The method of any previous embodiment, wherein synaptic activity is determined by: (a) the number of cells in the culture that express the second fluorophore and do not express the first fluorophore; and/or (b) spectral changes in a calcium (Ca²⁺)-sensitive dye or a voltage-sensitive dye.

A method for screening for a gene whose product enhances synaptic connectivity between retinal cells; the method comprising: incubating the in vitro retinal tissue of claim 43 under conditions such that the test gene is expressed; and testing for synaptic activity; wherein an increase in synaptic activity in cultures in which the test gene is expressed, compared to cultures in which the test gene is not expressed, indicates that the test gene encodes a product that enhances synaptic connectivity.

The method of any previous embodiment, wherein the retinal cells are PRs and second-order retinal neurons.

The method of any previous embodiment, wherein synaptic activity is determined by: (a) the number of cells in the culture that express the second fluorophore and do not express the first fluorophore; and/or (b) spectral changes in a calcium (Ca²⁺)-sensitive dye or a voltage-sensitive dye.

The method of any previous embodiment, wherein said conditions such that the test gene is expressed constitute culture in the presence of doxycycline.

The in vitro retinal tissue of any previous embodiment, wherein the cells comprise a mutation in the PDE6B gene.

The in vitro retinal tissue of any previous embodiment, wherein the cells comprise a mutation in the PDE6B gene.

A method for screening for a test substance that promotes survival of photoreceptor (PR) cells, the method comprising: (a) incubating the in vitro retinal tissue of claim 53 in the presence of the test substance; and (b) testing for PR cell survival; wherein an increase in PR cell survival in cultures in which the test substance is present, compared to cultures in which the test substance is not present, indicates that the test substance promotes survival of photoreceptor cells.

The method of any previous embodiment, wherein the test substance is selected from the group consisting of an exosome preparation, conditioned medium, a protein, a polypeptide, a peptide, a low molecular weight organic molecule, and an inorganic molecule.

The method of any previous embodiment, wherein the exosomes are obtained from a pluripotent cell.

The method of any previous embodiment, wherein the test substance is an epigenetic modulator.

The method of any previous embodiment, wherein the epigenetic modulator modulates a process selected from the group consisting of DNA methylation, DNA hydroxymethylation, histone methylation, histone acetylation, histone phosphorylation and histone ubiquitination. The method of any previous embodiment, wherein the epigenetic modulator modulates expression of a microRNA.

The method of any previous embodiment, wherein the test substance induces hypoxia.

A method for screening for a gene whose product promotes survival of photoreceptor (PR) cells, the method comprising: (a) culturing the in vitro retinal tissue of any previous embodiment under conditions such that the test gene is expressed; and (b) testing for PR cell survival; wherein an increase in PR cell survival in cultures in which the test gene is expressed, compared to cultures in which the test gene is not expressed, indicates that the test gene encodes a product that promotes survival of photoreceptor cells.

The method of any previous embodiment, wherein the test gene encodes a mitogen.

The method of any previous embodiment, wherein the test gene encodes a trophic factor.

The method of any previous embodiment, wherein the test gene encodes an epigenetic modulator.

The method of any previous embodiment, wherein the epigenetic modulator modulates a process selected from the group consisting of DNA methylation, DNA hydroxymethylation, histone methylation, histone acetylation, histone phosphorylation and histone ubiquitination.

The method of any previous embodiment, wherein the epigenetic modulator modulates expression of a microRNA.

The method of any previous embodiment, wherein the test gene encodes a product that induces hypoxia.

The method of any previous embodiment, wherein PR cell survival is determined by the number of cells in the culture that express the second fluorophore and do not express the first fluorophore.

The method of any previous embodiment, wherein PR cell survival is determined by spectral changes in a calcium (Ca²⁺)-sensitive dye or a voltage-sensitive dye.

The method of any previous embodiment, wherein said conditions such that the test gene is expressed constitute culture in the presence of doxycycline.

The method of any previous embodiment, wherein the steps are in the order described.

1. In vitro retinal tissue, wherein the retina tissue:
(a) comprises a disc-like three-dimensional shape; and
(b) comprises a concentric laminar structure comprising one or more of the following cellular layers extending radially from the center of the structure:
(i) a core of retinal pigmented epithelial (RPE) cells,
(ii) a layer of retinal ganglion cells (RGCs),
(iii) a layer of second-order retinal neurons (inner nuclear layer),
(iv) a layer of photoreceptor (PR) cells, and
(v) a layer of retinal pigmented epithelial cells.

2. The in vitro retinal tissue of claim 1, wherein any one or more of the layers comprises a single cell thickness.

3. The in vitro retinal tissue of claim 1, wherein any one or more of the layers comprises a thickness greater than a single cell.

4. The in vitro retinal tissue of claim 1, wherein any one more of the layers further comprises progenitors to the cells in the layer.

5. The in vitro retinal tissue of claim 1, wherein one or more of the cells express LGR5.

6. The in vitro retinal tissue of claim 1, wherein one or more of the cells express one or more genes selected from the group consisting of RAX, OTX2, LHX2, CHX10, MITF, PAX6, CRX, Recoverin (RCVRN) and BRN3A.

7. The in vitro retinal tissue of claim 1, wherein one or more of the cells express one or more of the SOX1, SOX2, OTX2 and FOXG1 genes.

8. The in vitro retinal tissue of claim 1, wherein one or more of the cells express one or more of the RAX, LHX2, SIX3, SIX6 and PAX6 genes.

9. The in vitro retinal tissue of claim 1, wherein one more of the cells express one or more of the NEURO-D1, ASCL1 (MASH1), CHX10 and IKZF1 genes.

10. The in vitro retinal tissue of claim 1, wherein one more of the cells express one or more genes selected from the group consisting of CRX, RCVRN, NRL, NR2E3, PDE6B, and OPN1SW.

11. The in vitro retinal tissue of claim 1, wherein one more of the cells express one or more genes selected from the group consisting of MATH5, ISL1, BRN3A, BRN3B, BRN3C and DLX2.

12. The in vitro retinal tissue of claim 1, wherein one more of the cells express one or more genes selected from the group consisting of PROX1, PRKCA, CALB1 and CALB2.

13. The in vitro retinal tissue of claim 1, wherein one more of the cells express one or more genes selected from the group consisting of MITF, TYR, TYRP, RPE65, DCT, PMEL, Ezrin and NHERF1.

14. The in vitro retinal tissue of claim 1, wherein one or more of the cells do not express the NANOG and OCT3/4 genes.

15. The in vitro retinal tissue of claim 1, wherein the cells do not express markers of endoderm, mesoderm, neural crest, astrocytes or oligodendrocytes.

16. A composition comprising the in vitro retinal tissue of claim 1.

17. The composition of claim 16, further comprising a hydrogel.

18. The composition of claim 16, wherein the composition is a cell culture.

19. The cell culture of claim 18, wherein culture is conducted under adherent conditions.

20. The cell culture of claim 18, further comprising a hydrogel.

21-72. (canceled)
